(19) United States
(12) Patent Application Publication
(10) Pub. No.: US 2025/0255828 A1
(43) Pub. Date: Aug. 14, 2025

(54) LYOPHILIZED LIPID NANOPARTICLES AND METHODS OF THEIR USE

(71) Applicant: Ultragenyx Pharmaceutical Inc., Novato, CA (US)

(72) Inventors: ISAIAS PRADO, Novato, CA (US); AKHILESH BHAMBHANI, Novato, CA (US); BENJAMIN CHENG, Novato, CA (US); HAIRUI ZHANG, Novato, CA (US); XIAOWEI WANG, Hillsborough, CA (US); VIVEK KUMAR GARRIPELLI, Novato, CA (US)

(73) Assignee: Ultragenyx Pharmaceutical Inc., Novato, CA (US)

(21) Appl. No.: 19/047,781

(22) Filed: Feb. 7, 2025

Related U.S. Application Data
(60) Provisional application No. 63/551,205, filed on Feb. 8, 2024.

(51) Int. Cl. A61K 9/51; A61K 9/19; A61K 31/7105; A61K 38/17; A61K 38/45; A61K 38/47

(52) U.S. Cl. CPC A61K 9/5123; A61K 9/19; A61K 31/7105; A61K 38/1709; A61K 38/45; A61K 38/47

(57) ABSTRACT

The invention is directed to the field of therapeutic formulations, in particular to lyophilization of a therapeutic cargo molecule, such as RNA. The invention provides a method for lyophilization of a molecule. The present disclosure further describes a lyophilized composition obtainable by the inventive method, a pharmaceutical composition, a vaccine, a therapeutic and a kit or kit of parts. Moreover, the disclosure herein provides a novel lyophilization excipient that protects the composition from degrading when, for example, lyophilizing RNA. The use of the inventive method further includes the manufacture of a composition that can be used after lyophilization with equivalent therapeutic effect and composition integrity.

### CROSS-REFERENCE TO RELATED APPLICATIONS

This application claims the benefit of and priority to U.S. Provisional Application No. 63/551,205, filed 8 Feb. 2024, the entire disclosure of which is hereby incorporated herein by reference for all purposes.

### BACKGROUND OF THE INVENTION

Transfection of nucleic acid therapeutics and other agents has been accomplished by encapsulating the active molecules in lipid nanoparticles. Drawbacks of this methodology include the inability to store compositions for later use because of degradation of the nanoparticles or the encapsulated contents therein. For example, compositions of lipid nanoparticles that encapsulate nucleic acid molecules may be stable for only a few minutes or hours at 25° C., and only a few days or weeks at 4° C. Further drawbacks include the need for very low temperature storage of the lipid nanoparticle compositions.

One way to provide for long-term storage of a therapeutic composition is to prepare a lyophilized form, which can be stored and reconstituted to provide a formulation for administration of the therapeutics.

However, it has not been possible in general to generate lyophilized forms of lipid nanoparticles containing nucleic acid agents, so that the lipid nanoparticle can be regenerated with the nucleic acid agent encapsulated to form a stable formulation. The lyophilization process can destroy the nanoparticles and/or the nucleic acid agents. Some methods have involved chemically attaching protective groups or components to the lipid nanoparticles, or to the nucleic acid agent, which is disadvantageous. Other methods may use liposomes as an alternative to LNP, without providing for encapsulation and/or stability of the nucleic acid agents.

There is a continuing need for compositions and methods to provide lyophilized forms of nanoparticles that can be reconstituted with favorable properties, including transgene activity or potency, particle size, storage stability, and shelf-life to deliver various nucleic acid agents.

What is needed are methods, compositions and compounds for forming stable solutions or suspensions of lipid nanoparticles that can be stored in solid lyophilized forms, where the nanoparticles encapsulate a cargo molecule or composition.

In gene therapy as well as in many other therapeutically relevant biochemical and biotechnological applications such as enzyme replacement therapies, nucleic acids are used for therapeutic and diagnostic purposes. As an example, rapid progress has occurred in recent years in the field of gene therapy and promising results have been achieved. Nucleic acids are therefore regarded as important tools for gene therapy and prophylactic and therapeutic vaccination against, for instance, genetic disorders as well as infectious and malignant diseases. Gene therapy has also shown to be an effective therapeutic against uncommon diseases that affect a small portion of the population (rare and ultra-rare diseases).

Nucleic acids, both DNA and RNA as well as small antisense oligonucleotides (ASOs), have been used widely in gene therapy, either in naked or in complexed form. In this context, the application of nucleic acids and particularly of RNA for therapeutic treatment of genetic disorders is becoming ever-increasingly important.

The application of RNA represents a favored tool in modern molecular medicine, which also exhibits some superior properties over the application of DNA. As generally known, transfection of DNA molecules may lead to serious complications. For example, application of DNA molecules bears the risk that the DNA integrates into the host genome. Integration of foreign DNA into the host genome can have an influence on the expression of host genes and can trigger the expression of an oncogene or the inactivation of a tumor suppressor gene. Furthermore, an essential gene—and, as a consequence, the product of such an essential gene—may also be inactivated by the integration of the foreign DNA into the coding region of the gene.

The result of such an event may be particularly dangerous if the DNA is integrated into a gene, which is involved in regulation of cell growth. Notwithstanding the risks associated with its application, DNA still represents an important tool. However, these risks do not occur if RNA, particularly mRNA, is used instead of DNA. An advantage of using RNA rather than DNA is that no virus-derived promoter element has to be administered in vivo and no integration into the genome may occur. Furthermore, the RNA, in order to exert its function, does not need to overcome the barrier to the nucleus. However, a main disadvantage of the use of RNA is its instability. For instance, RNA is susceptible to hydrolysis by ubiquitous ribonucleases or by divalent cations and is typically rapidly degraded, e.g., after a few hours or days in solution. Rapid degradation occurs even in the absence of RNases, e.g., when RNA is stored in solution at room temperature for a few hours or days. To avoid such rapid degradation, RNA (in solution) is typically stored at −20° C. or even −80° C. and under RNAse free conditions to prevent degradation of the RNA. Such storage conditions, however, do not sufficiently prevent a loss of function over time. Additionally, applying such conditions is very cost-intensive, especially for shipping and storage, e.g., whenever such low temperatures have to be guaranteed. Thus, there is a need for stable biological therapeutics using, for example, siRNA, mRNA, or other RNA-based modalities.

One further method for stabilization of RNA comprises lyophilization or freeze-drying of the RNA. Lyophilization is a method known and recognized in the art, which is used to enhance storage stability of temperature sensitive biomolecules. During lyophilization, a solvent, such as water, is typically removed from a frozen sample via sublimation. However, a nominal amount of water may remain.

The process of lyophilization is usually characterized by a primary and a secondary drying step. During the primary drying step, free, i.e., unbound, water surrounding the biomolecule and optionally further components, sublimes from the frozen solution. Subsequently, water, which is bound by the biomolecule on a molecular basis, may be removed in a secondary drying step by adding thermal energy. In both cases, the hydration sphere around the biomolecule is lost.

During lyophilization, a sample containing a biomolecule is initially cooled below the freezing point of the solution and accordingly of the water contained therein. As a result, the water freezes. Depending, amongst other parameters, on temperature, cooling rate (freezing rate), and the time for freezing, crystals may be formed. This exerts physical stress on the biomolecule and other components of the solution, which may lead to damage of the biomolecule such as—in the case of a nucleic acid—breakage of strands, structural alteration, etc. Furthermore, due to the decrease of volume and loss of the hydration sphere, autocatalytic degradation processes are favored, e.g., by traces of transition metals. In addition, the concentration of traces of acids and bases can result in significant changes of the pH value. Lyophilization involves two types of stress, namely freezing and drying. Both types of stress are known to damage nucleic acid molecules.

In order to reduce or minimize damage to the molecule a number of cryoprotectants are discussed for lyophilization purposes to prevent these damages. In this context, cryoprotectants are understood as excipients, which allow influencing the temperature characteristics of the mixture. Cryoprotectants can also partially or totally replace the hydration sphere around a molecule and may thus at least partially prevent catalytic and hydrolytic processes. Lyophilization may increase the stability of DNA under long-term storage, but may also cause some damage due to the initial lyophilization process, potentially through changes in the DNA secondary structure, breaks of the nucleic acid chain(s) or the concentration of reactive elements such as contaminating metals.

One solution for improving the storage capabilities of lipid nanoparticle formulations is to manufacture the lipid nanoparticle formulations as a lyophilized product that can be subsequently reconstituted prior to administration. Lyophilized lipid nanoparticle compositions can be stored at more practical temperatures, allowing more convenient modes of distribution and administration.

Although lyophilization technologies have existed for several decades, the application of these technologies does not translate well to lipid nanoparticle formulations, which lose several of their desired properties including low polydispersity, small particle size, high percentage of encapsulation, and in vivo efficacy upon reconstitution after conventional lyophilization techniques. Therefore, improved formulations and methods are needed to allow for initial lyophilization processes to maintain structural integrity of the encapsulated cargo molecule and also maintain therapeutic effectiveness upon reconstitution.

### BRIEF SUMMARY OF THE INVENTION

The present disclosure provides lyophilized compositions and methods of making lyophilized compositions.

The disclosure is directed to the field of therapeutic formulations, in particular to lyophilization of a therapeutic cargo molecule, such as nucleic acid agents (e.g., mRNA). The disclosure provides methods for lyophilization of a cargo molecule encapsulated inside a lipid nanoparticle (LNP). The present disclosure further describes lyophilized compositions obtainable by the disclosed methods, such as pharmaceutical compositions, vaccines, and other therapeutics. Moreover, the disclosure herein provides a novel approach to produce mRNA/LNP solution pre-lyophilization that protects the composition from degrading when, for example, lyophilizing RNA. The use of the disclosed methods further includes the manufacture of a composition that can be used after lyophilization with equivalent therapeutic effect and composition integrity.

### BRIEF DESCRIPTION OF DRAWINGS

FIG. 1 is a graphical illustration of intensity weighted particle distribution.

FIG. 2 is a graph showing Small-angle X-ray Scattering (SAXS) patterns of the measurement samples.

FIG. 3 is a cartoon depiction of proposed sample of a lipid nanoparticle containing mRNA.

FIG. 4A is a process flow chart for preparing a sample for lyophilization.

FIG. 4B is a graphical illustration of size and PDI under various buffer formulations.

FIG. 5 is a flow chart showing an mRNA lyophilization production protocol.

FIG. 6 is a graphical illustration of lyophilization cycle profile for Round 1 screening completion of primary drying process.

FIG. 7 is a graphical illustration of lyophilization cycle profile for Round 2 screening completion of primary drying process.

FIG. 8 is a graphical illustration of lyophilization cycle profile for Round 3 screening completion of primary drying process.

FIG. 9 shows supplemental data of Round 1 samples pre-lyophilization.

FIG. 10 shows supplemental data of Round 1 samples post-lyophilization.

FIG. 11 shows supplemental data of Round 1 samples reconstituted with Milli-Q water.

FIG. 12 shows supplemental data of Round 2 samples pre-lyophilization.

FIG. 13 shows supplemental data of Round 2 samples post-lyophilization.

FIG. 14 shows supplemental data of Round 2 samples reconstituted with Milli-Q water.

FIG. 15 shows supplemental data of Round 2 samples reconstituted with TNS Buffer.

FIG. 16 shows supplemental data of Round 3 samples pre-lyophilization.

FIG. 17 shows supplemental data of Round 3 samples post-lyophilization.

FIG. 18 shows supplemental data of Round 3 samples reconstituted with Milli-Q water.

FIG. 19 shows size stability results observed for lots 230523A and B stored 1 week at 40° C., and 6 months at 25° C., 5° C. and −20° C.

FIG. 20 shows stability study results for lots 230523A and B by polydispersity index [PDI].

FIG. 21 shows stability results by encapsulation efficiency observed for lots 230607A and B after 1 week at 40° C., 1 weeks at 25° C., and 6 months at 5° C. and −20° C.

FIG. 22 shows stability results by encapsulation efficiency for lots 230523A and B.

FIG. 23 shows purity study results for lots 230607A and B after 2 weeks at 40° C. and 25° C., and 6 months at 5° C. and −20° C.

FIG. 24 shows purity study results for lots 230523A and B after 7 days at 40° C., and 6 months at 25° C., 5° C., and −20° C.

### DETAILED DESCRIPTION OF THE INVENTION

The present disclosure provides lyophilization methods that result in a preservation of lipid nanoparticle integrity, integrity of the encapsulated molecule, the particle size of the lipid nanoparticles within an acceptable degree of pre-lyophilized particle size, with an improved excipient formulation. The methods stem from the discovery that specific combinations of excipients can be added to a pretreated suspension of the mRNA and eventually to nanoparticles prior to subjecting the suspension to a lyophilization process. In addition, lyophilization parameters are employed in combination with these excipients to achieve high quality lyophilized lipid nanoparticle product. The lyophilized product is easily reconstituted and readily administered as a pharmaceutical.

### Definitions

“Cargo molecule” means peptides or proteins, DNA, RNA, carbohydrates, lipids or chemically devised molecules of natural or non-natural origin, such as metals, dyes, and nanoparticles that can be encapsulated and/or delivered as a carrier for a therapeutic agent and/or a therapeutic agent. Non-limiting examples of cargo molecules include DNA, RNA, miRNA, mRNA, siRNA, synergistic activation mediator (SAM), circular RNA, ASO, or a combination thereof.

The term DLS as used herein means Dynamic Light Scattering.

“Effective” refers to capability of a therapeutic or diagnostic agent, or an amount thereof (e.g., mass, volume, dosage, concentration, and/or time period) to achieve one or more desired result(s).

“Encapsulation efficiency” (EE) as used herein is the fraction of initial drug that is encapsulated by the nanoparticles (NPs).

“Excipient” as used herein refers to an organic or inorganic ingredient, natural or synthetic inactive ingredient in a formulation, with which one or more active ingredients are combined.

“Nanoparticle” as used herein refers to a lipid containing particle that can be formed using a T-mixing approach, microfluidic approach, jet-mixing, solvent emulsion, spray drying, or precipitation in bulk or microfluids, wherein the solvent is removed to no more than an insignificant residue, leaving an electron dense particle of size ≤200 nm, preferably ≤150 nm, ≤100 nm (which may, or may not, be hollow or have a liquid filled interior such as a liposome) polymeric particle, unlike a micelle whose form is dependent upon being present in an aqueous solution.

“N/P: Molar ratio” is defined as the ratio of nitrogen on ionizable lipid to Phosphate on the nucleic acid backbone.

“Prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

The term “protein” “polypeptide” or “peptide” refers to a natural or synthetic molecule comprising two or more amino acids linked by the carboxyl group of one amino acid to the alpha amino group of another.

The term Rₕ as used herein means Hydrodynamic Radius.

The term R_{g} as used herein means Radius of Gyration.

The term SAM refers to synergistic activation mediator that is an engineered protein complex for the transcriptional activation of endogenous genes.

The term SAXS as used herein means Small-angle X-ray Scattering.

The present disclosure provides improved lyophilized compositions and methods of making lyophilized compositions. Specifically, the present disclosure is directed to the field of molecule formulations, in particular lyophilization of a molecule, such as a nucleic acid agent. The disclosure provides methods for lyophilization of a molecule. The present disclosure further describes lyophilized compositions obtainable by the disclosed methods, pharmaceutical compositions, vaccines, and other therapeutics. Moreover, the disclosure herein provides a novel lyophilization excipient that protects the composition from degrading when, for example, undergoing the process of lyophilization. The use of the disclosed methods further includes the manufacture of a composition that can be used after lyophilization with equivalent therapeutic effect and composition integrity.

As described in further detail herein, the method of making a composition comprises a LNP and a cargo molecule encapsulated within the LNP. In exemplary embodiments, the composition is capable of being lyophilized. The method includes combining an aqueous solution of the cargo molecule and an ionizable lipid composition contained in an organic solvent solution, thereby forming a lipid/cargo molecule suspension.

In example embodiments, the cargo molecule that is combined with the lipid composition comprises at least one nucleic acid, such as DNA or RNA. In related embodiments, the cargo molecule comprises micro RNA (miRNA), messenger RNA (mRNA), short interfering RNA (siRNA), short hairpin RNA (shRNA), antisense oligonucleotide (ASO), synergistic activation mediator (SAM), circular RNA, or a combination thereof.

The lipid composition can include an ionizable lipid composition used to form an ionizable organic solution. In these embodiments the ionizable lipid composition comprises an ionizable cationic lipid, such as a tertiary amine that is capable of deprotonation under neutral conditions and is positively charged in pH conditions below the acid-dissociation constant (pKₐ) of the lipid. In other embodiments, the ionizable organic solvent solution comprises at least one of an ionizable lipid, DSPC, cholesterol, PEG-DMG and combinations thereof. In some related embodiments, the ionizable organic solvent solution comprises a N/P molar ratio of ≥2.5, ≥6.0, or ≥9.0. It should be appreciated that the combining the aqueous solution of the cargo molecule and a lipid composition with an ionizable organic solvent solution forms a lipid/cargo molecule suspension.

In example embodiments, excipients are added to the lipid/cargo molecule suspension. In other related embodiments, the method of making the composition comprises excipients where the excipients are added to the aqueous solution of the cargo molecule prior to being combined with the lipid composition. In at least these example embodiments, the excipients are selected from a group comprising citrate, tris, arginine, NaCl, sucrose, PVA, phosphate, HEPES, trehalose, KCl, acetate, bis-tris, histidine, glucose, lactose, raffinose, alanine, asparagine, proline, glutamic acid, methionine, threonine, and combinations thereof. In other example embodiments, excipient(s) comprise at least one positively charged and at least one negatively charged compound or an amino acid combination. The excipients that can be used within the scope of the invention, include but are not limited to at least two amino acids selected from Ala, Arg, His, Asp, Pro, Glu, Met, Thr, and combinations thereof. In other embodiments, the excipient comprises a hydrophilic polymeric compound. In other related embodiments, the excipient comprises a plasticizer.

In example embodiments, the aqueous solution and/or lipid composition comprises an organic solvent. Examples of organic solvents within the scope of the present disclosure include at least one of ethanol, DMSO, acetone of other alcohol containing DSPC, cholesterol, PEG-DMG, an ionizable lipid, and combinations thereof. In the methods of making the lipid/cargo molecule suspension, the process comprises diluting the lipid/cargo molecule suspension that is combined with the organic solvent to lower the concentration of organic solvent to ≤10% of the total composition. In alternative embodiments involving the methods of making the lipid/cargo molecule suspension, the process comprises diluting the lipid/cargo molecule suspension that is combined with the organic solvent to lower the concentration of organic solvent to ≤3% of the total composition.

The dilution step comprises a first dilution step to increase the pH to about 6 and a second dilution step to increase the pH to between 6.8 and 7.8. The first dilution step comprises at least one of phosphate, HEPES, tris, bis-tris, acetate, citrate, NaCl, KCl, glutamic acid, arginine, histidine, methionine, glucose, sucrose, lactose, trehalose, raffinose, and combinations thereof. The second dilution buffer comprises at least one buffer compound selected from amino acids, sugars, salts and combinations thereof. These dilutions steps may further comprise the step of adding a stabilizer to the aqueous solution of the cargo molecule.

The stabilizer is added prior to mixing the cargo molecule with the organic solvent. The stabilizer is optionally present in any one of the at least one dilution steps.

When forming the lipid/cargo molecule suspension, the pH of the composition is increased to at or above 5.0. When modifying the pH, the adjustment to at or above a pH of 5.0 can be performed before the dilution step (b). In other alternative embodiments, when modifying the pH, the adjustment to at or above a pH of 5.0 can be performed after the dilution step (b). In either pH modification step being performed prior to, or after the dilution step, the pH can be adjusted to at least 6. In other embodiments, the pH is adjusted to between 6.9 and 7.9.

As described above, once the lipid/cargo molecule suspension is formed, the lipid/cargo molecule suspension can undergo the process of lyophilization. In these embodiments lyophilizing a composition comprises the lipid nanoparticle having the cargo molecule encapsulated therein, thereby forming a lyophilized cargo molecule/LNP, the method comprising freezing the lipid/cargo molecule suspension. Once the freezing step is completed, a further drying step can be performed on the frozen lipid/cargo molecule suspension. During the drying process, the drying step can be performed by a process selected from a group comprising spin-freeze drying, continuous freeze-drying, sublimation, desorption, vacuum foam drying, microwave vacuum drying, spray drying, evaporation and combinations thereof. During the drying step, the target sublimation is achieved when the moisture is present at an amount of ≤5%.

Once lyophilization is completed, the cargo molecule/LNP is tested for potency and/or purity yield. Potency and/or purity yield of the lyophilized composition is ≥70%, ≥80%, ≥90%, or ≥95% when compared to the cargo molecule/LNP prior to lyophilization. Other characteristics of the lyophilized cargo molecule/LNP include the determination of particle size and/or encapsulation efficiency. Particle size undergoing the process of lyophilization described herein, includes; <100 nm, <90 nm, <80 nm, or <75 nm, while encapsulation efficiency of the lyophilized cargo molecule/LNP is about ≥80%, or about ≥90%.

In some exemplary embodiments, the cargo molecule within the cargo molecule/LNP comprises a therapeutic agent at a starting concentration of ≤2 mg/ml under acidic conditions with a pH under 5, preferably between pH 3-4.5. In some exemplary embodiments, the cargo molecule within the cargo molecule/LNP comprises mRNA as the therapeutic agent at a starting concentration of ≤1 mg/ml under acidic conditions with a pH under 5, preferably between pH 3-4.5. In some exemplary embodiments, the cargo molecule within the cargo molecule/LNP comprises mRNA as the therapeutic agent at a starting concentration of <0.55 mg/ml under acidic conditions with a pH under 5, preferably between pH 3 and pH 4.5. In some exemplary embodiments, the cargo molecule within the cargo molecule/LNP comprises mRNA as the therapeutic agent at a starting concentration of ≤0.22 mg/ml under acidic conditions with a pH under 5, preferably between pH 3 to pH 4.5. In an example embodiment, a cargo molecule/LNP comprises mRNA encoding amylo-alpha-1, 6-glucosidase, 4-alpha-glucanotransferase (AGL), wherein the mRNA encoding AGL is codon-optimized. In some embodiments, the AGL is human AGL (i.e. hAGL). Example cargo molecules that can be utilized within the scope of the present disclosure can be found in U.S. Pat. No. 11,377,643, the contents of which are incorporated herein in its entirety. These cargo molecules can be used for ameliorating, preventing, delaying onset, or treating a disease or condition associated with reduced activity of amylo-alpha-1, 6-glucosidase, 4-alpha-glucanotransferase (AGL) in a subject. Other cargo molecules can be used to treat diseases, such as progressive familial intrahepatic cholestasis (PFIC), including PFIC-1, PFIC-2 and PFIC-3. For example, a cargo molecule/LNP may comprise a mRNA encoding human bile salt export pump (human BSEP). The mRNA may be an ABCB11 mRNA encoding human BSEP. The mRNA may be codon-optimized.

In the described process of forming the cargo molecule/LNP compositions, the cargo molecule, in a non-lyophilized state, prior to encapsulation, is dissolved in a buffer under acidic pH conditions below the pKa of the lipid composition. The buffer may optionally comprise a salt between 25 mM and 100 mM. In some embodiments, the salt is NaCl. The buffer may optionally comprise 50 mM NaCl.

The process of lyophilization of the present disclosure provides an improvement in cargo molecule/LNP integrity, such that the step of reconstitution of the lyophilized cargo molecule/LNP composition can be performed with a diluent.

The lipid nanoparticle in the composition after being lyophilized and then reconstituted is increased in size between 3-18% or to a size ≤18% relative to the size of the lipid nanoparticle prior to lyophilization. In other embodiments, the lipid nanoparticle in the composition after being lyophilized and then reconstituted is increased in size no more than 18% relative to the size of the lipid nanoparticle prior to lyophilization.

Prior to lyophilization, the defined cargo molecule, where the cargo molecule is RNA, has an encapsulation efficiency (EE) of the RNA of at least 70%, 80%, 85%, or 90%. Importantly, the encapsulation efficiency (EE) of the composition after being reconstituted has an average variance of no more than 20% of the lyophilized or aqueous form of the composition. In other embodiments, the encapsulation efficiency (EE) of the composition after being reconstituted has an average variance of between 10-20% of the lyophilized or aqueous form of the composition.

The methods of forming a lyophilized composition can also include the process of preparing a lyophilized composition comprising lipid nanoparticles with reduced percentage of water in a lipid nanoparticle (LNP) core, the method comprising:
(a) combining an aqueous solution of a cargo molecule with a lipid composition, thereby forming a lipid/cargo molecule suspension;
(b) mixing the lipid/cargo molecule suspension with an organic solvent;
(c) adding an excipient, wherein the excipient is added before or after the aqueous solution of the cargo molecule is combined with the lipid composition;
(d) diluting the lipid/cargo molecule suspension having the organic solvent to lower the concentration of organic solvent to ≤10% of the total composition by volume; thereby forming a lipid nanoparticle composition comprising lipid nanoparticles comprising a shell of the lipid composition and a core comprising the cargo molecule in aqueous solution with the organic solvent;
(e) lyophilizing the lipid nanoparticle composition; and
(f) drying the lipid nanoparticle composition, thereby forming the lyophilized composition with lipid nanoparticles having reduced percentage of water in the lipid nanoparticle core.

In some embodiments, the process of reducing water in the LNP core as described herein can reduce the moisture content of the lyophilized composition to ≤5% by weight. Moreover, when performing step (f), the drying step can be performed by a process selected from spin-freeze drying, continuous freeze-drying, sublimation, desorption, vacuum foam drying, microwave vacuum drying, spray drying, evaporation, and combinations thereof. The drying step of at least this embodiment occurs at a temperature between 30° C. to −50° C. The drying step is generally performed at a time range of between 10-3000 minutes.

The process may further comprise reconstituting the lyophilized composition in a diluent. The diluent, in some embodiments, includes saline, water, 50-500 mM NaCl, arginine, D5W, sucrose, polyvinyl alcohol (PVA), tris, or any combinations thereof.

Samples (see Table 1) were prepared using the prescribed methods and were analyzed using small-angle X-ray Scattering (SAXS) measurement on three different samples including 1 control (220316) and 2 samples (210825) & (1119A). Buffer was also measured for background subtraction. SAXS results suggests that 220316 forms Lipid Nanoparticles (LNPs) with random lipid distribution in the core and with PEG lipids on the surface of LNP. Following the introducing of mRNA, 1119A and 210825 show bilayer structure, which represents the interaction between the lipids and mRNA. Bilayer stacks were also observed in SAXS measurements which is consistent with TEM images (not shown).

TABLE 1
Samples received

| Sample Number | Sample ID # | Sample Description |
|---|---|---|
| 1 | 220316 | Control |
| 2 | 1119A | Sample |
| 3 | 210825 | Sample |
| 4 | 220808 | Buffer |

Sample preparation is performed by DLS measurement using a Zetasizer Ultra Red system with a side detector mode (90°). SAXS measurements were performed using an Empyrean Nano system with a ScatterX78 SAXS accessory with a PIXCel 3D detector. Approximately 40 μL of sample were loaded in a 1.0 mm (O.D.) quartz capillary tube. The covered scattering vector, (q≡4π/λ sin θ/2 where θ is the scattering angle and A is the incident X-ray wavelength=1.5418 Å) is from ˜0.006 Å⁻¹ to 0.5 Å⁻¹. Sample scattering and buffer scattering were measured in the same capillary tube for proper background subtraction. EasySAXS software was used for background subtraction which utilized the attenuated incident beam intensity as the normalization factor. SAXS data analysis is performed on SASView 5.0.5.

For sample 220316, control, a combined Core-shell model with a polymer chain model was used to fit the SAXS pattern. For samples 210825 and 1119A, a combined core 2 shell model with a Lorentzian peak. A combined 2 core shell model, as used herein is described in Small 18, no. 9 (2022) by Cui et al, entitled “Mechanistic studies of an automated lipid nanoparticle reveal critical pharmaceutical properties associated with enhanced mRNA functional delivery in vitro and in vivo”.

### DLS Measurement

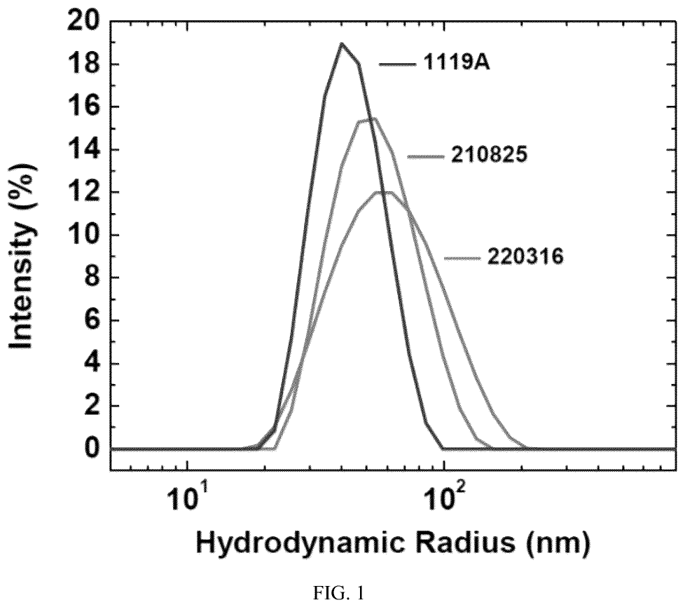

Referring now to FIG. 1, DLS was performed to verify the sample freshness after shipping. The intensity weighted particle size distribution results are shown. The Z-averaged hydrodynamic radius (Rₕ) as well as polydispersity index (PDI) are listed in Table 2. The Rₕ measured by Malvern Panalytical (MP) was within the required specification. Therefore, the SAXS measurements were performed.

TABLE 2
Hydrodynamic radius and PDI for each sample

| Sample | R_{H} MP (NM) | R_{H} UX (NM) | PDI MP | PDI UX |
|---|---|---|---|---|
| 220316 | 52.5 ± 1.2 | 38 | 0.19 ± 0.01 | 0.139 |
| 210825 | 48.7 ± 1.4 | 40.0 | 0.15 ± 0.02 | 0.125 |
| 1119A | 40.5 ± 0.1 | 36.5 | 0.08 ± 0.03 | 0.08 |

### SAXS Measurement

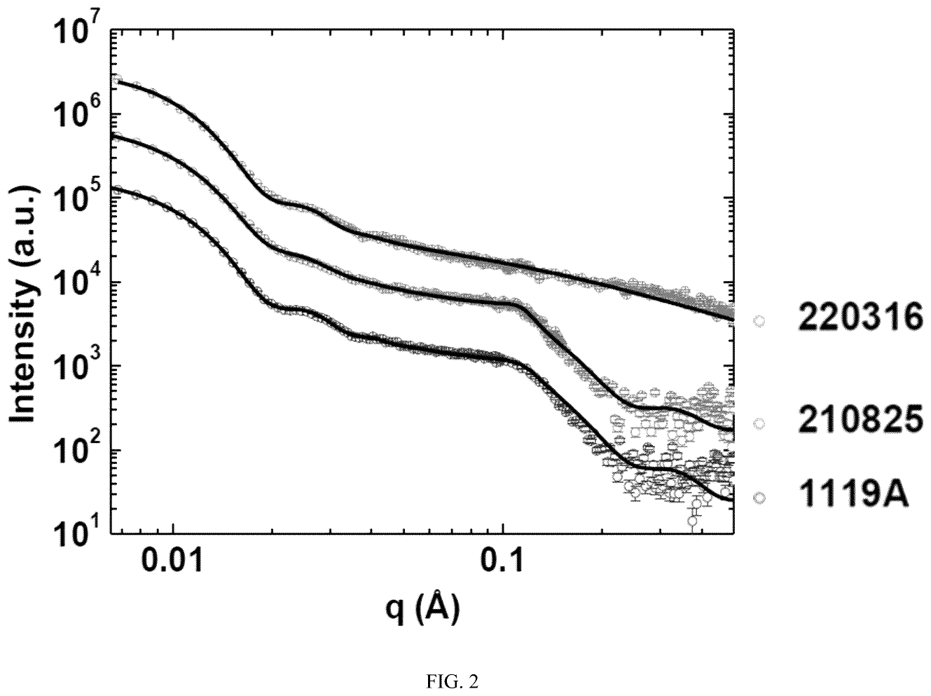

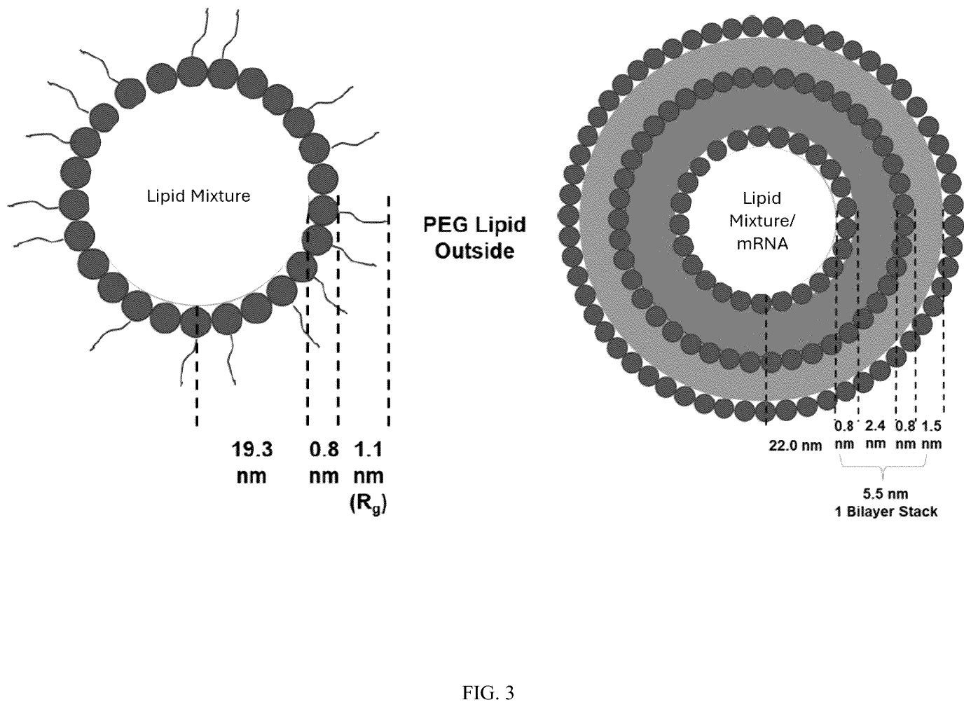

SAXS was performed and the results are shown in FIG. 2. The fitting parameters are listed in Table 3. For all the samples measured, the size is comparable and sample 1119A and 210825 has the largest and smallest PDI, respectively. For sample 220316, since no mRNA is added, no lipid bilayer (phosphate-alkyl chain-phosphate) is formed. The core of the 220316 is a lipid mixture while the PEG-lipid was primarily located at the surface with PEG faced to the solvent/buffer forming a PEG shell. The proposed LNP structure of sample 220316 is shown in FIG. 3(a). The structural parameters were also listed where radius of the core is ˜ 19 nm and the radius of gyration of PEG is ˜ 1 nm. Once lipids are mixed with mRNAs, i.e. sample 210825 and 1119A, the bilayer structure is formed.

In addition, these bilayers further form stacks which include water layers as shown in FIG. 3(b) where the small circles represent phosphate group with alkyl chain and water layer as yellow and blue area. Here, one stack is defined as phosphate-alkyl chain-phosphate-water. Therefore, in FIG. 3(b) the proposed structure has 1 stack plus one phosphate. The ratio between diffraction peak and the core-shell structure reveals the number of bilayer stacks. The electron density in the bilayer stack is higher in 210825. Moreover, the electron density is larger in the bilayer stack than 1119A. Based on the RiboGreen assay, results were indicative that the electron density in the bilayer stack is higher in 210825.

SAXS results confirmed the bilayer structure formed with presence of mRNA. In addition, the structural parameters can be extracted from the SAXS pattern by fitting the data. Moreover, SAXS results are consistent with the TEM images and the RiboGreen assay. Referring still to FIG. 3 (Proposed structure from Small Angle X-ray Scattering (SAXS)), analysis suggests the presence of bilayer stacks with water in between them. The small circles represent phosphate group with alkyl chain and water layer illustrated as yellow and blue area. While not wanting to be bound by any particular theory, a skilled artisan, in view of the detailed description herein, could determine that replacing water with stabilizers during the mRNA/LNP process early on would be essential to enable successful lyophilization, e.g., excipients that serve as a water replacer (e.g. sucrose and trehalose) and/or known for charge shielding (e.g. NaCl) and Arg/Glu salt pairs. The desired ranges were then determined empirically, and it was unexpectedly found that the presence of Glu/Arg, in presence or absence of His, performed better then Arginine alone or in absence of any amino acid. Moreover, the packing density defect during the dehydration process manifested as an increase in particle size upon reconstitution. Thus, minimizing shock (due to charge and/or osmotic imbalances) during reconstitution may enable retention of particle size post-lyophilization. In prior lyophilization protocols, in-use stability for non-lyophilized formulations (i.e. frozen liquid) revealed that diluting out stabilizer resulted in dramatic change in size and loss in recovery post reconstitution.

TABLE 3
Fitting parameters used for SAXS analysis

| | Core | Shell 1 | | Shell 2 | Peak | Bilayer | Scale |
|---|---|---|---|---|---|---|---|
| | Radius | Thickness | Polymer | Thickness | Position | Spacing | CS/Scale |
| Sample | (NM) | (NM) | Rₓ (NM) | (NM) | (Å⁻¹) | (NM) | Peak |
| 220316 | 19.3 | 1.0 | 1.1 | N/A | N/A | N/A | N/A |
| | | (Fixed) | | | | | |
| 210825 | 22.0 | 2.4 | N/A | 1.0 | 0.115 | 5.5 | 0.56 |
| | | (Fixed) | | (Fixed) | | | |
| 1119A | 22.6 | 2.4 | N/A | 1.0 | 0.115 | 5.5 | 0.42 |
| | | (Fixed) | | (Fixed) | | | |

The methods described herein provide an improved method of successfully lyophilizing mRNA/LNP compositions with retention of mRNA purity, relative potency, % EE and particle size with low polydispersity index (PDI) as measured using Capillary Electrophoresis (CE), In-cell Western (ICW), RiboGreen Assay and Dynamic Light Scattering (DLS).

It should be appreciated that limited shelf-life of mRNA/LNP is driven in part by presence of water in the LNP core that results in, for example, hydrolysis of phosphodiester bond in the mRNA backbone and lyophilization serves as an appealing method to improve the shelf-life of mRNA/LNP. Additionally, LNPs are self-assembled multi-complex systems and stresses induced during the freeze-drying (such as freezing, vacuum, dehydration, etc.) processes are known to impact the physicochemical properties (i.e. particle size, PDI, EE % etc.) and ultimately mRNA-LNP functionality (i.e. potency or efficacy). Moreover, the addition of stabilizers early during the process, starting with mRNA pre-mix, and optionally optimizing the composition may serve as a means to overcome the challenges associated with dehydration of mRNA/LNP using freeze-drying. Stabilizers such as sucrose and trehalose serve as water replacers; however, their use in mRNA pre-mix has not been observed before during mRNA/LNP process. Similarly, the presence of additional stabilizer that can form salt bridges such as Glu+Arg as part of mRNA dilution buffer have not been observed before. Surprisingly, the presence of stabilizer such as sucrose, glutamate and arginine in mRNA dilution buffer favorably reduced the particle size to <70 nm (versus the original process with a particle size of ˜80 nm).

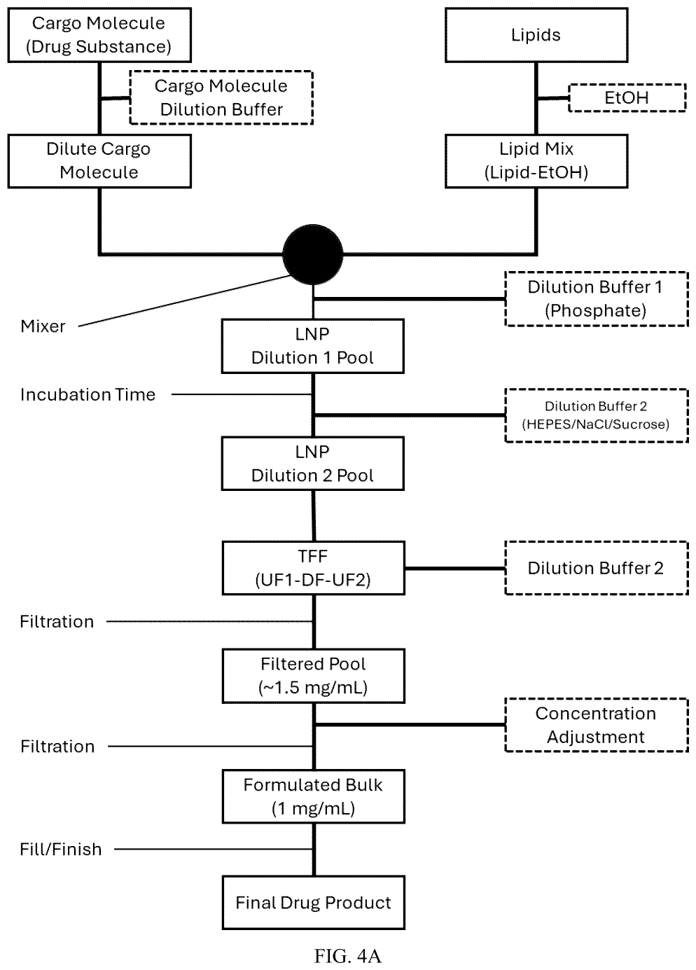

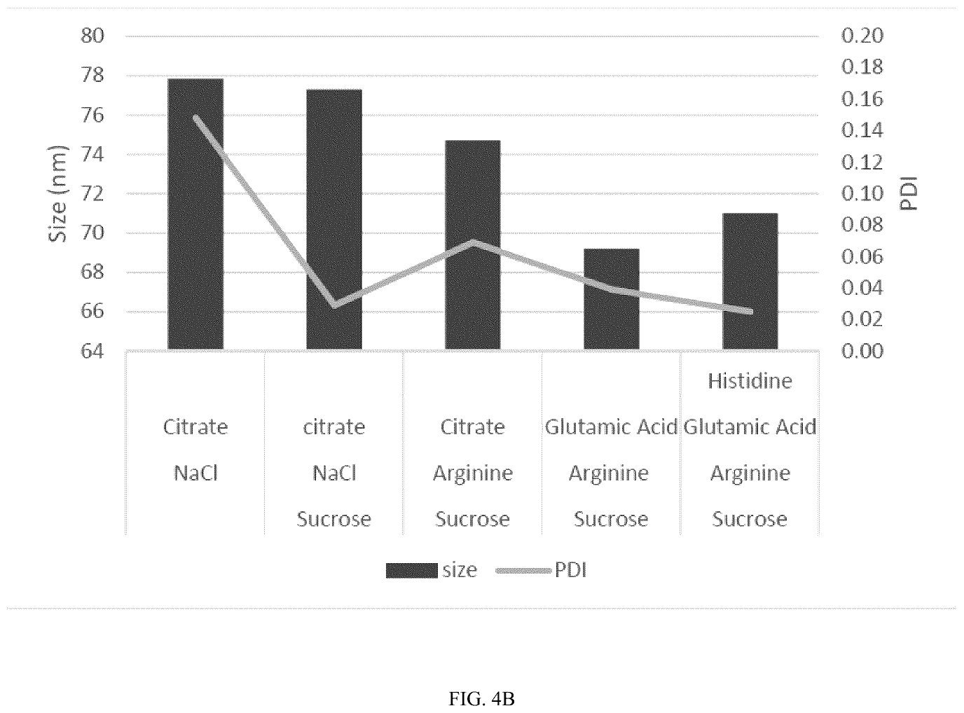

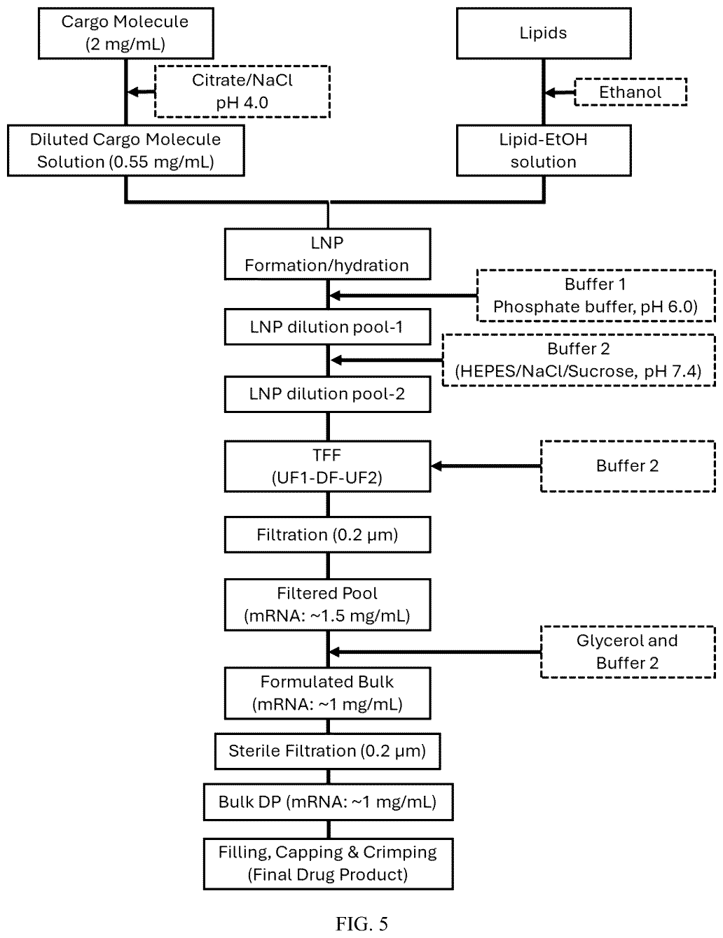

As shown in FIGS. 4A and 4B (see also Table 4), two different approaches to LNP production prior to lyophilization are shown. In the traditional approach (referred to as pC-1 in Table 4), mRNA/LNP are fabricated and lyoprotectants are added prior to lyophilization. In the alternate approach (referred to as pC-2 in Table 4), selected cryoprotectants and lyoprotectants are introduced in the mRNA dilution buffer itself. The bar graph (FIG. 4B) demonstrates that presence of Glu+Arg+Sucrose provides lowest particle size prior to lyophilization while keeping PDI <0.1.

TABLE 4
Approaches to LNP production

| Step | pC-1 | pC-2 |
|---|---|---|
| mRNA Dilution Buffer | 6.5 mM Citrate, 50 mM | 5 mM Glutamic Acid, 5 mM |
| | NaCl, pH 4.0 | Histidine, 25 mM Arginine, |
| | | 15% Sucrose, pH 4.0 |
| Dilutions Buffer 1 | 45 mM Phosphate, pH 6.0 | 50 mM Histidine, 9% |

Sucrose, pH 6.0

| Dilution Buffer 2 | | 100 mM Tris, 15% Sucrose, pH 7.6 |
|---|---|---|
| Diafiltration Buffer | | 50 mM Tris, 15% Sucrose, pH 7.4 |
| Concentration | Excipient and | 50 mM Tris, 500 mM Arginine, 250 mM NaCl, 15% |
| Adjustment Buffer 1 | Concentration | Sucrose, 0.5% PVA, pH 7.4 |
| | Adjustment | |
| | Concentration | 50 mM Tris, 100 mM Arginine, 50 mM NaCl, 15% |
| | Adjustment | Sucrose, 0.1% PVA, pH 7.4 |

While not reciting any single factor as being critical, several factors are required to minimize the impact of freeze-drying on mRNA/LNP physicochemical (such as size, PDI, EE % etc.) and functional properties (based on % purity, potency, etc.) such as: (a) Process of making mRNA/LNP by ensuring presence of stabilizers during (mixing, dilution step 1, dilution step 2, lyophilization once mRNA/LNPs are formed, and reconstitution); (b) Approach to mixing with microfluidics yielding more consistent particles than T-mixer; (c) Starting mRNA concentration wherein lower starting concentration of mRNA results in lower particle size pre-lyophilization; (d) Presence of salt wherein [NaCl]≥25 mM is needed maintain particle <80 nm; (e) Presence of Glu+Arg+sucrose yields particle size <80 nm; (f) Lyophilization process parameters also impacted particle size and corresponding potency and purity results; (g) Presence of Tris and Sucrose by itself is not sufficient to keep particle size <100 nm and presence of Arg+PVA10+NaCl is needed with earlier addition of excipients (pre-mixing and/or dilution 1: PC2), preferred over late addition (after dilution step 2; PC1); and (h) Custom reconstitution media such as formulation buffer, saline, half saline performed better than water alone in retaining particle size.

Stabilization of mRNA/LNP remains a challenge, as exemplified by cold-chain storage requirement of COVID vaccines. Liquid formulations are preferred from a customer convenience perspective; however, they pose sterility assurance and stability challenges often requiring cold-chain storage. Lyophilization serves to overcome the stability challenges and in early-stage clinical program provides an approach for early entry into clinic by attaining stable product with flexibility on dosing and drug concentration by using custom reconstitution media and volume. Thus, there remains a consensus in the field that freezing and lyophilization are the most common approach to retain long-term storage and shelf life of lipid nanoparticles. As discussed above, the stresses induced by lyophilization often results in a dried mRNA/LNP of suboptimal characteristics. Under certain conditions lyophilization of mRNA/LNP results in a suboptimal particle size (often ≥100 nm) with PDI, often ≥0.1 or a loss of EE (in some cases ≤90%) or a compromise on potency and/or purity. Comparative lyophilization processes, such as those found in US 2022/0047519 documents the use of specialized excipient to retain potency. Similarly, the addition of 30% ethanol during the reconstitution step results in a size comparable to fresh LNPs, however, that process is neither convenient nor practical for use in the clinic (see Ball et al, Int J Nanomedicine 2016 December 30:12:305-315.doi: 10.2147/IJN.S123062. Use of trehalose and sucrose seems to alleviate the problem; however, the particle size was high (˜200 nm) with low EE (60-70%). Similarly, previous studies (see Muramatsu, Mol Ther. 2022 May 4; 30 (5): 1941-1951)) demonstrated a particle size of ˜96-99 nm for lyophilized and reconstituted Luc mRNA/LNPs, while the latter reference demonstrates particle size of lyophilized LNP to be greater than 150 nm.

The present disclosure exemplifies the process of making stable lyophilized mRNA/LNP formulations and their resulting composition of matter using Test samples as cargo molecule/LNP composition. Test sample is a lipid encapsulated therapeutic mRNA expressing a gene of interest (GOI) formulated at 1 mg/mL in 50 mM HEPES, 50 mM NaCl, 9% Sucrose, 5% Glycerol pH 7.4 and stored as frozen liquid stable formulation. Two different approaches are shown using multiple compositions. In the first approach (referred to as pC-1), mRNA/LNP are fabricated and lyoprotectants are added prior to lyophilization. In the alternate approach (referred to as pC-2), selected cryo- and lyoprotectants are introduced in the mRNA dilution buffer itself.

TABLE 5
pC-1 and pC-2 mRNA-LNP lyophilization

| | | Size | | | PDI | | | Encapsulation | | | |
|---|---|---|---|---|---|---|---|---|---|---|---|
| | DP | Pre | Post | | Pre | Post | | Pre | Post | | |
| | Formulation | Lyo | Lyo | Δ | Lyo | Lyo | Δ | Lyo | Lyo | Δ | Potency |
| pC-1 | 50 mM Tris, | 83 | 95 | 11 | 0.135 | 0.111 | −0.024 | 98% | 95% | −3% | NA |
| | 100 mM | | | | | | | | | | |
| | Arg, 0.1% | | | | | | | | | | |
| | PVA10, | | | | | | | | | | |
| | 50 mM | | | | | | | | | | |
| | NaCl, 9% | | | | | | | | | | |
| | Sucrose, pH | | | | | | | | | | |
| | 7.4 | | | | | | | | | | |
| | 20 mM Tris, | 83 | 97 | 14 | 0.110 | 0.130 | 0.020 | 98% | 96% | −2% | NA |
| | 100 mM | | | | | | | | | | |
| | Arg, 0.1% | | | | | | | | | | |
| | PCA10, | | | | | | | | | | |
| | 50 mM | | | | | | | | | | |
| | NaCl, 9% | | | | | | | | | | |
| | Sucrose, pH | | | | | | | | | | |
| | 7.4 | | | | | | | | | | |
| | 50 mM Tris, | 83 | 92 | 9 | 0.116 | 0.131 | 0.015 | 98% | 94% | −4% | NA |
| | 100 mM | | | | | | | | | | |
| | Arg, 0.1% | | | | | | | | | | |
| | PVA10, | | | | | | | | | | |
| | 25 mM | | | | | | | | | | |
| | NaC1, 9% | | | | | | | | | | |
| | Sucrose, pH | | | | | | | | | | |
| | 7.4 | | | | | | | | | | |
| pC-2 | 50 mM Tris, | 71 | 101 | 30 | 0.037 | 0.101 | 0.064 | 99% | 86% | −13% | 222% |
| | 9% Sucrose, | | | | | | | | | | |
| | pH 7.4 | | | | | | | | | | |
| | 50 mM Tris, | 71 | 95 | 24 | 0.049 | 0.117 | 0.068 | 99% | 87% | −12% | 191% |
| | 15% | | | | | | | | | | |
| | Sucrose, pH | | | | | | | | | | |
| | 7.4 | | | | | | | | | | |
| | 50 mM Tris, | 71 | 83 | 12 | 0.034 | 0.068 | 0.034 | 99% | 95% | −4% | 164% |
| | 100 mM | | | | | | | | | | |
| | Arg, 0.1% | | | | | | | | | | |
| | PVA10, | | | | | | | | | | |
| | 50 mM | | | | | | | | | | |
| | NaCl, 9% | | | | | | | | | | |
| | Sucrose, pH | | | | | | | | | | |
| | 7.4 | | | | | | | | | | |
| | 50 mM Tris, | 71 | 82 | 11 | 0.031 | 0.065 | 0.034 | 99% | 94% | −5% | 144% |
| | 100 mM | | | | | | | | | | |
| | Arg, 0.1% | | | | | | | | | | |
| | PVA10, | | | | | | | | | | |
| | 50 mM | | | | | | | | | | |
| | NaCl, 15% | | | | | | | | | | |
| | Sucrose, pH | | | | | | | | | | |
| | 7.4 | | | | | | | | | | |

mRNA/LNP size pre-lyophilization is lower when excipients are introduced early in mRNA dilution buffer (pC-2) versus (pC-1). For the similar process pC-2, the presence of Arginine, PVA10 and NaCl enables greater retention of particle size upon lyophilization. When final formulations were optimized, the formulation included 50 mM Tris, 100 mM Arginine, 0.1% PVA10, 50 mM NaCl, 9% Sucrose, and a pH of 7.4 for both the process condition (i.e. pC-1 and pC-2, respectively).

In alternative embodiments, mRNA dilution buffer components, such as salt, can be altered to further improve the invention described herein. For example, effects of [NaCl] on Particle Characteristics: mRNA diluted buffer ranged from 5-75 mM NaCl. After 30 min. incubation period between Dilution 1 Pool and Dilution 2 Pool, the LNPs were evaluated for size, PDI, and encapsulation. PDI is lowest when [NaCl] is between 35-50 mM NaCl (See Table 6 below). As shown, the effect of salt concentration of mRNA/LNP particle size, PDI and encapsulation efficiency demonstrates that [NaCl]≥25 mM results in acceptable mRNA/LNP particle size.

TABLE 6
mRNA dilution buffer components

| [NaCl] in mRNA | | | Encapsulation |
|---|---|---|---|
| Dilution Buffer (mM) | Size (nm) | PDI | Efficiency (%) |
| 75 | 81 | 0.088 | 97 |
| 62.5 | 78 | 0.121 | 98 |
| 50 | 77 | 0.069 | 97 |
| 35 | 73 | 0.038 | 96 |
| 25 | 79 | 0.156 | 99 |
| 15 | 96 | 0.190 | 98 |
| 5 | 140 | 0.184 | 95 |

In other related embodiments, modifications of Amino acids: mRNA/LNP size follows the order Glu/Arg (+His)<Arg alone<no amino acid can also be used to provide improvements in LNP/cargo molecule compositions. In other related embodiments, sugars, such as sucrose, can be used to show improvement in size and PDI.

In related embodiments, the modification of an alcohol can also be used to modify the EE. As shown in Table 7, the percentage of ethanol during mixing is shown. Particle characteristics when % EtOH ranges from 10-30% are stable (5% Ethanol condition has slightly lower encapsulation). As % EtOH decreases, mRNA must be decreased to maintain the N: P ratio. Thus, mRNA/LNP are stable when % EtOH ranges from 10-30% while 5% EtOH condition results in a slightly lower encapsulation efficiency.

TABLE 7
Modification of alcohol content

| | | | Encapsulation | Hydration Time | |
|---|---|---|---|---|---|
| % EtOH | Size (nm) | PDI | Efficiency (%) | (1 g Batch) | |
| 30 | 81 | 0.108 | 99 | 6.7 | min |
| 25 | 77 | 0.069 | 97 | 8.1 | min |
| 20 | 75 | 0.062 | 97 | 10.1 | min |
| 15 | 72 | 0.049 | 95 | 13.5 | min |
| 10 | 79 | 0.035 | 92 | 20.2 | min |
| 5 | 79 | 0.053 | 89 | 40.4 | min |

As shown, lower starting concentration results in lower particle size pre-lyophilization (e.g. 0.22 mg/ml Vs. 0.55 mg/ml). The data from Table 7 also indicates that the lower final concentration pre-lyophilization correlates with smaller change in particle size (relative to same final formulation at higher concentration). Excipient concentration ranges can include 20-100 mM Tris, 0-200 mM Arginine, 0.035-0.2% PVA10, 25-50-100-300 mM NaCl, 5-30% Sucrose and a pH of about 7.4; Reconstitution media: Normal Saline is preferred for reconstitution, however, (1) formulation buffer comprising of 50 mM HEPES, 50 mM NaCl, 9% Sucrose, 5% Glycerol pH 7.4 at 1×, 0.5× or at 0.25× concentration, (2) water, (3) D5W, (4) 0.15 M Arginine, (5) 1× or 0.5× Saline (6) 0.5 M NaCl or (7) 50 mM Tris 50 mM NaCl, 9% Sucrose, pH 7.4 at 1×, 0.5× or 0.25× may also be used in a manner such that pre-lyophilization and post-lyophilization mRNA concentration are in 1:1, 2:1 or 4:1 ratio.

mRNA/LNP characterization

| | | | |
|---|---|---|---|
| Formulation (pC-2) | 50 mM HEPES, | 50 mM HEPES, | 50 mM Tris, |
| | 100 mM Arginine, | 100 mM Arginine, | 100 mM Arginine, |
| | 0.1% PVA10, | 0.1% PVA10, | 0.1% PVA10, |
| | 50 mM NaCl, | 50 mM NaCl, | 50 mM NaCl |
| | 9% Sucrose pH 7.4 | 9% Sucrose pH 7.4 | 15% Sucrose |
| | (Run1- Control) | (Run 2-Control) | pH 7.4 |

| | | Pre- | Post- | | Pre- | Post- | | Pre- | Post- | |
|---|---|---|---|---|---|---|---|---|---|---|
| Reconstitution | | Lyo | Lyo | Delta | Lyo | Lyo | Delta | Lyo | Lyo | Delta |
| Normal | Size (nm) | 84 | 103 | 19 | 82 | 98 | 16 | 70.87 | 80.43 | 10 |
| Saline (2:1 | PDI | 0.072 | 0.106 | 0.034 | 0.069 | 0.098 | 0.036 | 0.031 | 0.044 | 0.013 |
| concentration) | Encapsulation (%) | 98.70% | 90.40% | −8.30% | 98.90% | 93.90% | −4.10% | 98.70% | 92.00% | −6.60% |
| Normal Saline | Size (nm) | 84 | 103 | 19 | 82 | 97 | 15 | 70.87 | 82 | 11 |
| (1:1 Conc.) | PDI | 0.072 | 0.088 | 0.016 | 0.069 | 0.086 | 0.024 | 0.031 | 0.065 | 0.034 |
| | Encapsulation (%) | 98.70% | 91.90% | −6.80% | 98.90% | 94.10% | −3.90% | 98.70% | 93.80% | −4.90% |

| | | | | | | |
|---|---|---|---|---|---|---|
| Water (2:1 | Size (nm) | 84 | 130 | 46 | NA | NA |
| conc.) | PDI | 0.072 | 0.119 | 0.047 | NA | NA |
| | Encapsulation (%) | 98.70% | 88.10% | −10.60% | NA | NA |

| 0.5M NaCl | Size (nm) | NA | 82 | 99 | 17 | NA |
|---|---|---|---|---|---|---|
| (2:1 conc.) | PDI | NA | 0.069 | 0.103 | 0.041 | NA |
| | Encapsulation (%) | NA | 98.90% | 94.80% | −3.20% | NA |
| | Size (nm) | NA | 82 | 99 | 17 | NA |
| 0.15M | PDI | NA | 0.069 | 0.107 | 0.045 | |

| Arginine | Encapsulation (%) | NA | 98.90% | 94.60% | −3.40% | NA |
|---|---|---|---|---|---|---|
| (2:1 conc.) | | | | | | |

| | | | | | | |
|---|---|---|---|---|---|---|
| D5W (2:1 | Size (nm) | 84 | 117 | 33 | NA | NA |
| conc.) | PDI | 0.072 | 0.122 | 0.05 | NA | NA |
| | Encapsulation (%) | 98.70% | 90.60% | −8.10% | NA | NA |

Lyophilization - pC-1

| | | | | | | Chamber |
|---|---|---|---|---|---|---|
| | | | | Ramp Rate | | Pressure |
| Step | Temperature (° C.) | Time | | (° C./min) | | (mTorr) |
| Loading | 20° C. | NA | | NA | | NA |
| Freezing | 20° C. to −50° C. | 70 | min | 1° | C./min | NA |
| | −50° C. | 120 | min | NA | | NA |
| | −50° C. to −30° C. | 200 | min | 0.1° | C./min | NA |
| | −30° C. | 5 | min | NA | | NA |
| Primary Drying | −30° C. | 2800 | min | NA | | 100 |
| Secondary Drying | −30° C. to 25° C. | 550 | min | 0.1° | C./min | 100 |

| | | | | |
|---|---|---|---|---|
| 25° C. | 300 | min | NA | 100 |

| | | | | |
|---|---|---|---|---|
| Storage | 5° C. | NA | NA | 100 |

Lyophilization - pC-2

| | | | | | | Chamber |
|---|---|---|---|---|---|---|
| | | | | Ramp Rate | | Pressure |
| Step | Temperature (° C.) | Time | | (° C./min) | | (mTorr) |
| Loading | 18° C. | 5 | min | NA | | NA |
| Freezing | 18° C. to 5° C. | 13 | min | 1° | C./min | NA |
| | 5° C. | 30 | min | NA | | NA |
| | 5° C. to −50° C. | 45 | min | 1° | C./min | NA |
| | −50° C. | 180 | min | NA | | NA |
| Primary Drying | −50° C. to −30° C. | 200 | min | 0.1° | C./min | 100 |
| | −30° C. | 1070 | min | NA | | 100 |
| | −30° C. to −25° C. | 50 | min | 0.1° | C./min | 100 |
| | −25° C. | 1200 | min | NA | | 100 |
| Secondary Drying | −25° C. to 25° C. | 500 | min | 0.1° | C./min | 100 |

| | | | | |
|---|---|---|---|---|
| 25° C. | 600 | min | NA | 100 |

The studies described herein examine optimal excipients and conditions that can be used to form cargo molecule/LNP within the scope of the invention described herein. Table 8 provides a list of chemicals and materials used to formulate and analyze the therapeutic cargo molecules.

TABLE 8
Chemical and materials list

| Item | Vendor | Catalog Number |
|---|---|---|
| HEPES | Sigma-Aldrich | H3375-100G |
| Schott borosilicate 2 cc, 13 mm serum glass vials | West Pharmaceutical | 68000386 |
| | Services | |
| WPS 4432/50 Grey, B2-40, stoppers | West Pharmaceutical | 19700004 |
| | Services | |
| Vivaspin 20, 30 kDa MWCO PES centrifugal | Sartorius | 26-9323-61 |
| concentrators | | |
| Slide-A-Lyzer, 20 kDa MWCO dialysis cassettes, | Thermo Scientific | 66003 |
| 0.5-3.0 mL | | |
| Slide-A-Lyzer, 20 kDa MWCO dialysis cassettes, | Thermo Scientific | 66012 |
| 3-12 mL | | |
| Polysorbate 80 (PS80) | Croda | SR40925 |
| Poloxamer 188 (F-68) | Spectrum | P1169 |
| Sucrose | JT Baker | 4074-05 |
| Sorbitol | EMPROVE | 1.11597.9023 |
| Potassium Sorbate | Sigma-Aldrich | 85520-50G |
| Sodium Benzoate | Sigma-Aldrich | 18106-1KG-R |
| Sodium Phosphate Monobasic Monohydrate | Fisher | S468-500 |
| Trehalose | Pfanstiehl | T-10404 |
| Mannitol | JT Baker | 2553-05 |
| EDTA-Na2 | JT Baker | 8995-01 |
| Dextran 40 | Spectrum | DE124 |
| Poly(vinyl alcohol) | Sigma-Aldrich | 348406-25g |
| L-Arginine | Sigma-Aldrich | A8094-1KG |
| L-Glutamic Acid | JT Baker | M746-07 |
| L-Histidine | JT Baker | 2080-06 |
| L-Methionine | Sigma-Aldrich | M5308-100G |
| Cis-4-Hydroxy-D-proline | Sigma-Aldrich | H5877 |
| L-Proline | Sigma-Aldrich | P5607-100G |
| L-Threonine | VWR | E808 |
| Glacial Acetic Acid | EMD | AX0073-9 |
| Sodium Hydroxide | Macron | 7680-06 |
| BSA | Sigma-Aldrich | A2153 |
| 60% Iodixanol, Optiprep Density Gradient Medium | Sigma-Aldrich | D1556-250 mL |
| RNase Free Water | Ambion | AM9932 |
| 10% TX-100 | Sigma-Aldrich | 93443-100 ML |
| RiboGreen Reagent | Invitrogen | R11490A |
| Ribo Ruler High Range RNA Ladder | Thermo Fisher | SM1821 |
| Quant-iT ™ RiboGreen ™ RNA Assay Kit | Invitrogen | R11490 |
| RNase Zap | Thermo Fisher | AM9780 |
| 3000 Series Nanosphere ™ Size Standards, 100 nm | Thermo Scientific | 3100A |

Excipients and excipient formulations were evaluated to determine how to best (1) lyophilize the LNP composition and (2) maintain a high level of fidelity between the pre-lyophilized and post-lyophilized LNP composition.

The evaluation study included various excipients to stabilize cargo molecule through lyophilization. The physical stability of cargo molecule was evaluated in three rounds of screening by analytical methods previously verified as effective stability-indicating assays for this product. These studies analyze the following: visual appearance, dynamic light scattering (DLS), and RiboGreen RNA quantitation.

For lyophilization, formulations containing 9% sucrose, 50 mM NaCl, and 50 mM Tris-HCl consistently displayed elegant, lyophilized cakes across all three rounds. RNase-free water reconstitution of sucrose formulations containing arginine and PVA10 displayed the least opalescence. Across the three rounds, DLS results following lyophilization revealed that formulations containing 9% sucrose showed smaller increases in hydrodynamic diameters compared to other non-sucrose formulations. The inclusion of arginine and PVA10 further reduced the increases in hydrodynamic diameters observed in DLS results. RiboGreen analysis concluded that reconstitution with RNase-free water yielded greater RNA percent encapsulation than reconstitution with TNS (Tris, NaCl, and sucrose) buffer. Despite experiencing smaller losses of encapsulation, iodixanol-containing formulations exhibited large increases in hydrodynamic diameters. Furthermore, despite displaying the most elegant cakes, Dextran 40 formulations were the most opalescent of the reconstituted formulations in Round 3, displayed the largest loss of encapsulation, and exhibited the greatest increase in hydrodynamic diameters. Notably, formulations containing arginine, PVA10, and NaCl showed high percentages of RNA encapsulation after reconstitution, reinforcing their beneficial effects on stability as observed in DLS and visual appearance.

In summary, the formulation of 9% sucrose, 50 mM NaCl, 50 mM Tris-HCl, 100 mM arginine, and 0.05% PVA10 presents superior stabilization of test sample through lyophilization and subsequent reconstitution with water, compared to other formulations tested. The stabilization benefits of these excipients may be further corroborated with the analysis of test sample's biological activity.

An Excipient Screening Study for test sample was conducted to assess excipients that stabilize test sample at 0.25 mg/mL during lyophilization. The active pharmaceutical ingredient (API) examined in this study was an mRNA cargo molecule expressing a GOI. The material used for this study was comprised of the following:
Excipient Screening, Round 1, Drug Product (DP), 1 mg/mL in 50 mM HEPES, 50 mM NaCl, 9%, Sucrose, 5% Glycerol, pH 7.4, Lot 1119-A.
Excipient Screening, Round 2 Drug Product (DP), Stored at −70° C., 0.99 mg/mL in 50 mM HEPES, 50 mM NaCl, 9% Sucrose, 5% Glycerol, pH 7.4, Lot 210113.
Excipient Screening, Round 3 Drug Product (DP), Stored at −70° C., 1.00 mg/mL in 50 mM HEPES, 50 mM NaCl, 9% Sucrose, 5% Glycerol, pH 7.4, Lot 210825, Stored at −70° C.

In the Excipient screening study, the following parameters were fixed:
(1) Fill volume: 0.1 mL/well in a 96-well plate
(2) API Concentration: 0.25 mg/mL
(3) pH: 7.4

The following excipients were examined in each round of the screening (Tables 9-11):
Round 1: HEPES, Tris-HCl, polymers, hydrophobic salts, amino acids, NaCl, sucrose, and iodixanol.
Round 2: Various concentrations of PVA10, P188, arginine, proline, sucrose, and iodixanol.
Round 3: EDTA, methionine, Dextran 40, various concentrations of Tris-HCl, arginine, PVA10, sucrose, and NaCl.

TABLE 9
Excipient Screening Matrix (Round 1)

| | Buffer | | Hydrophobic salts/AA | | |
|---|---|---|---|---|---|
| Form # | (50 mM) | Polymers | (100 mM) | NaCl | Sucrose/Iodixanol |
| 1 | HEPES | 0.05% | Sodium Benzoate | 50 mM | None |
| 2 | | PVA10 | Potassium Sorbate | | |
| 3 | | 0.2% | Sodium Benzoate | | |
| 4 | | P188 | Potassium Sorbate | | |
| 5 | Tris-HCl | 0.05% | Potassium Sorbate | | |
| 6 | | PVA10 | Sodium Benzoate | | |
| 7 | | 0.2% | Potassium Sorbate | | |
| 8 | | P188 | Sodium Benzoate | | |
| 9 | HEPES | 0.05% | Potassium Sorbate | None | |
| | | PVA10 | | | |
| 10 | | 0.2% | Sodium Benzoate | | |
| | | P188 | | | |
| 11 | Tris-HCl | 0.05% | Sodium Benzoate | | |
| | | PVA10 | | | |
| 12 | | 0.2% | Potassium Sorbate | | |
| 13 | | P188 | None | 50 mM | 2% Sucrose |
| 14 | | | | | 9% Sucrose |
| 15 | | 0.05% | | | |
| | | PVA10 | | | |
| 16 | | 0.2% | Arginine | | None |
| 17 | | P188 | 100 mM Arginine + 100 | | |
| | | | mM Potassium Sorbate | | |
| 18 | | | 100 mM Proline + 100 mM | | |
| | | | Sodium Benzoate | | |
| 19 | HEPES | 0.05% | Proline | None | 9% Iodixanol |
| 20 | | PVA10 | Arginine | | |
| 21 | | 0.2% | Proline | | 9% Sucrose |
| 22 | | P188 | Arginine | | |
| 23 | Tris-HCl | 0.05% | Proline | | 9% Iodixanol |
| 24 | | PVA10 | Arginine | | |
| 25 | | 0.2% | Proline | | 9% Sucrose |
| 26 | | P188 | Arginine | | |
| 27 | | | Proline | 50 mM | 2% Sucrose |
| 28 | | | | | 20% Sucrose |
| 29 | | | Arginine | | 2% Sucrose |
| 30 | | | | | 9% Sucrose |

TABLE 10
Excipient Screening Matrix (Round 2)

| | Buffer | PVA10 | P188 | Amino | Amino Acid | Sucrose/ | NaCl |
|---|---|---|---|---|---|---|---|
| Form # | (50 mM) | (%) | (%) | Acid | Conc. (M) | Iodixanol (9%) | (mM) |
| 1 | Tris-HCl | 0.1 | 0.4 | Arginine | 0.05 | Sucrose | NA |
| 2 | Tris-HCl | 0.1 | 0.2 | Proline | 0.05 | Sucrose | NA |
| 3 | Tris-HCl | 0.05 | 0.4 | Proline | 0.1 | Sucrose | NA |
| 4 | Tris-HCl | 0.05 | NA | Arginine | 0.1 | Sucrose | NA |
| 5 | Tris-HCl | 0.035 | 0.2 | Proline | 0.1 | Sucrose | NA |
| 6 | Tris-HCl | 0.035 | NA | Proline | 0.1 | Sucrose | NA |
| 7 | Tris-HCl | 0.1 | 0.2 | Arginine | 0.05 | Iodixanol | NA |
| 8 | Tris-HCl | 0.1 | NA | Proline | 0.1 | Iodixanol | NA |
| 9 | Tris-HCl | 0.05 | 0.4 | Arginine | 0.1 | Iodixanol | NA |
| 10 | Tris-HCl | 0.05 | 0.2 | Proline | 0.05 | Iodixanol | NA |
| 11 | Tris-HCl | 0.035 | 0.4 | Proline | 0.05 | Iodixanol | NA |
| 12 | Tris-HCl | 0.035 | NA | Arginine | 0.05 | Iodixanol | NA |
| 13 | Tris-HCl | 0.1 | 0.4 | Arginine | 0.05 | Sucrose | 50 |
| 14 | Tris-HCl | 0.1 | 0.2 | Proline | 0.05 | Sucrose | 50 |
| 15 | Tris-HCl | 0.05 | 0.4 | Proline | 0.1 | Sucrose | 50 |
| 16 | Tris-HCl | 0.05 | NA | Arginine | 0.1 | Sucrose | 50 |
| 17 | Tris-HCl | 0.035 | 0.2 | Proline | 0.1 | Sucrose | 50 |
| 18 | Tris-HCl | 0.035 | NA | Proline | 0.1 | Sucrose | 50 |
| 19 | Tris-HCl | 0.1 | 0.2 | Arginine | 0.05 | Iodixanol | 50 |
| 20 | Tris-HCl | 0.1 | NA | Proline | 0.1 | Iodixanol | 50 |
| 21 | Tris-HCl | 0.05 | 0.4 | Arginine | 0.1 | Iodixanol | 50 |
| 22 | Tris-HCl | 0.05 | 0.2 | Proline | 0.05 | Iodixanol | 50 |
| 23 | Tris-HCl | 0.035 | 0.4 | Proline | 0.05 | Iodixanol | 50 |
| 24 | Tris-HCl | 0.035 | NA | Arginine | 0.05 | Iodixanol | 50 |
| 25 | Tris-HCl | NA | 0.2 | Proline | 0.1 | Iodixanol | 50 |
| 26 | Tris-HCl | NA | 0.2 | Arginine | 0.1 | Iodixanol | 50 |
| 27 | Tris-HCl | NA | NA | Proline | 0.1 | Iodixanol | 50 |
| 28 | Tris-HCl | NA | NA | Arginine | 0.1 | Sucrose | 50 |
| 29 | Tris-HCl | NA | NA | NA | NA | Iodixanol | 50 |
| 30 | Tris-HCl | NA | NA | NA | NA | Sucrose | 50 |

TABLE 11
Excipient Screening Matrix (Round 3)

| | Tris- | | | | | | | |
|---|---|---|---|---|---|---|---|---|
| | HCl | Arginine | PVA10 | | NaCl | EDTA | Methionine | Dextran |
| Form # | (mM) | (M) | (%) | Sucrose (%) | (mM) | (mM) | (mM) | 40 (%) |
| 1 | 50 | 0 | 0 | 9 | 50 | 0 | 0 | 0 |
| 2 | 50 | 0 | 0 | 3 | 50 | 0 | 0 | 0 |
| 3 | 100 | 0 | 0 | 9 | 50 | 0 | 0 | 0 |
| 4 | 200 | 0 | 0 | 9 | 50 | 0 | 0 | 0 |
| 5 | 50 | 0 | 0 | 9 | 150 | 0 | 0 | 0 |
| 6 | 50 | 0 | 0 | 9 | 300 | 0 | 0 | 0 |
| 7 | 50 | 0.1 | 0 | 9 | 50 | 0 | 0 | 0 |
| 8 | 50 | 0.2 | 0 | 9 | 50 | 0 | 0 | 0 |
| 9 | 50 | 0.1 | 0.05 | 9 | 50 | 0 | 0 | 0 |
| 10 | 50 | 0.2 | 0.05 | 9 | 50 | 0 | 0 | 0 |
| 11 | 200 | 0.1 | 0.05 | 9 | 50 | 0 | 0 | 0 |
| 12 | 100 | 0.1 | 0.05 | 9 | 50 | 0 | 0 | 0 |
| 13 | 50 | 0.1 | 0.05 | 9 | 150 | 0 | 0 | 0 |
| 14 | 50 | 0.01 | 0.05 | 9 | 300 | 0 | 0 | 0 |
| 15 | 50 | 0.01 | 0.05 | 3 | 50 | 0 | 0 | 0 |
| 16 | 50 | 0 | 0 | 9 | 50 | 0.1 | 50 | 1 |
| 17 | 50 | 0.1 | 0.05 | 9 | 50 | 0.1 | 50 | 1 |
| 18 | 50 | 0 | 0 | 9 | 50 | 0.1 | 50 | 0 |
| 19 | 50 | 0.1 | 0.05 | 9 | 50 | 0.1 | 50 | 0 |
| 20 | 50 | 0 | 0 | 9 | 50 | 0.1 | 0 | 0 |
| 21 | 50 | 0.1 | 0.05 | 9 | 50 | 0.1 | 0 | 0 |
| 22 | Positive Control Buffer | | | | | | | |
| 23 | 50 | 0 | 0 | 9 | 50 | 0 | 0 | 0 |
| 24 | 50 | 0 | 0 | 9 | 50 | 0 | 0 | 0 |
| 25 | 50 | 0.1 | 0.05 | 9 | 50 | 0 | 0 | 0 |
| 26 | 50 | 0.1 | 0.05 | 9 | 50 | 0 | 0 | 0 |
| 27 | 50 | 0 | 0 | 9 | 150 | 0 | 0 | 0 |
| 28 | 50 | 0 | 0 | 9 | 150 | 0 | 0 | 0 |
| 29 | 50 | 0.1 | 0.05 | 9 | 150 | 0 | 0 | 0 |
| 30 | 50 | 0.1 | 0.05 | 9 | 150 | 0 | 0 | 0 |

### Formulation Preparation

Two vials of the drug product (DP) in Round 1 and Round 2 and one vial of DP in Round 3 were removed from −70° C. storage and thawed at ambient temperature. The DP was pooled when necessary and mixed gently. In each round, a DP aliquot at 1.0 mg/mL in the current formulation (50 mM HEPES, 50 mM NaCl, 9% sucrose, 5% glycerol, pH 7.4) was taken as DP control and stored at 5° C. until analyzed with pre-lyophilized (pre-lyo) samples. The remaining DP in each round was then dialyzed against the respective dialysis buffers shown in Table 12. Dialysis was performed using Thermo Scientific™ Slide-A-Lyzer™, 20 kDa MWCO dialysis cassettes. Buffer exchange (≥10,000-fold) was completed over 22 hours at 5° C. Upon completion of the dialysis, the pH of the dialyzed bulk DP was confirmed, and the total RNA concentration of dialyzed DP was measured with RiboGreen analysis. The dialyzed DP was then diluted to 0.5 mg/mL with each round's corresponding dialysis buffer and filtered prior to dilution with 2× excipient stock solutions.

TABLE 12
Dialysis Buffers, DP Dialyzed, and Post-Dialysis pH

| | Formulation | | Post- | Post-Dialysis |
|---|---|---|---|---|
| | Buffer | Target | Dialysis | Total RNA |
| | (100 mM) | pH | pH | Conc. (mg/mL) |
| Round 1 | HEPES | 7.4 | 7.36 | 0.69 |
| | Tris | 7.4 | 7.38 | 0.60 |
| Round 2 | Tris | 7.4 | 7.52 | 0.58 |
| Round 3 | Tris | 7.4 | 7.36 | 0.62 |

### Excipient Screening Formulation

The 2× excipient stock solutions were prepared in water and filtered as shown in Table 13. The pH of the amino acid stock solutions was adjusted to pH 7.4. The 2× excipient stock solutions were then spiked into the dialyzed DP to reach the excipient parameters listed in Tables 9-11 at an 0.25 mg/mL concentration. Following preparation, 100 μL of each formulation was aliquoted as a pre-lyophilization control and was stored at 5° C.

TABLE 13
Excipient Stock Solutions

| Round 1 | | | | | Round 2 | | | Round 3 | | | | |
|---|---|---|---|---|---|---|---|---|---|---|---|---|
| Reagent | [Stock] | | [Target] | | Reagent | [Stock] | [Target] | Reagent | [Stock] | | [Target] | |
| Poloxamer | 20% | | 0.20% | | Poloxamer | 20% | 0.2% | Arginine | 1 | M | 0.01 | M |
| 188 | (w/v) | | | | 188 | (w/v) | 0.4% | | | | 0.1 | M |
| | | | | | | | | | | | 0.2 | M |
| PVA 10 | 4% | | 0.05% | | PVA 10 | 4% | 0.035% | PVA 10 | 4% | | 0.05% | |
| | (w/v) | | | | | (w/v) | 0.05% | | (w/v) | | | |
| | | | | | | | 0.1% | | | | | |
| Sodium | 2 | M | 0.1 | M | Sucrose | 60% | 9% | Sucrose | 60% | | 3% | |
| Benzoate | | | | | | (w/v) | | | (w/v) | | 9% | |
| Potassium | 2 | M | 0.1 | M | Iodixanol | 60% | 9% | NaCl | 2.5 | M | 0.05 | M |

| Sorbate | | | | | | | | | | | 0.15 | M |
|---|---|---|---|---|---|---|---|---|---|---|---|---|
| | | | | | | | | | | | 0.3 | M |
| Sucrose | 100% | 2% | Proline | 1 | M | 0.05 | M | EDTA | 0.01 | M | 0.1 | mM |
| | (w/v) | 9% | | | | 0.1 | M | | | | | |
| | | 20% | | | | | | | | | | |
| Iodixanol | 60% | 9% | Arginine | 1 | M | 0.05 | M | Methionine | 1 | M | 0.05 | M |

| | | | | | | | | 0.1 | M | | | |
|---|---|---|---|---|---|---|---|---|---|---|---|---|
| Proline | 1 | M | 0.1 | M | NaCl | 2.5 | M | 0.05 | M | Dextran 40 | 10% | 1% |

| | | | | | | | | | | | | |
|---|---|---|---|---|---|---|---|---|---|---|---|---|
| Arginine | 1 | M | 0.1 | M | — | — | — | Tris-HCl* | 1 | M | 0.1 | M |

| | |
|---|---|
| 0.2 | M |

| | | | | | | | | | | |
|---|---|---|---|---|---|---|---|---|---|---|
| NaCl | 2.5 | M | 0.05 | M | — | — | — | — | — | — |

### Lyophilization

Lyophilized samples were prepared by plating 100 μL/well of each DP formulation in a 96-well plate and the samples were lyophilized following the cycles shown in Table 14.
Primary drying was monitored for convergence of the Pirani Gauge and Capacitance Manometer. Convergences occurred ˜11.8 hours (Round 1) and ˜15.3 hours (Round 2) into the cycles.

TABLE 14
Parameters of Lyophilization

| | | | |
|---|---|---|---|
| | | | Chamber |
| Temp | Time | Ramp Rate | Pressure |
| (° C.) | (min) | (° C./min) | (mT) |

Round 1

| | | | | |
|---|---|---|---|---|
| Loading | 5 | N/A | N/A | N/A |
| *Freezing | 5 to−50 | 55 | 1 | N/A |
| | −50 | 120 | N/A | N/A |
| | −50 to −20 | 60 | 0.5 | N/A |
| | −20 | 120 | N/A | N/A |
| *Primary Drying | −20 | 1540* | N/A | 100 |
| Secondary Drying | −20 to 20 | 80 | 0.5 | 100 |
| | 20 | 600 | N/A | 100 |

Round 2

| | | | | |
|---|---|---|---|---|
| Loading | 5 | N/A | N/A | N/A |
| Freezing | 5 to −50 | 55 | 1 | N/A |
| | −50 | 120 | N/A | N/A |
| | −50 to −30 | 40 | 0.5 | N/A |
| | −30 | 120 | N/A | N/A |
| *Primary Drying | −30 | 1000* | N/A | 100 |
| Secondary Drying | −30 to 5 | 70 | 0.5 | 100 |
| | 5 | 1200 | N/A | 100 |

Round 3

| | | | | |
|---|---|---|---|---|
| Loading | 5 | N/A | N/A | N/A |
| Freezing | 5 to −50 | 55 | 1 | N/A |
| | −50 | 120 | N/A | N/A |
| | −50 to −30 | 40 | 0.5 | N/A |
| | −30 | 120 | N/A | N/A |
| *Primary Drying | −30 | 900** | N/A | 100 |
| Secondary Drying | −30 to 5 | 70 | 0.5 | 100 |
| | 5 | 1200 | N/A | 100 |

Primary drying was monitored for convergence of the Pirani Gauge and Capacitance Manometer. Convergences occurred ˜11.8 hours (Round 1) and ˜15.3 hours (Round 2) into the cycles.
   The secondary drying was manually advanced at <3m Torr differential of the Pirani Gauge and Capacitate Monometer (PVG/CM). Convergence occurred ˜16 hours into the cycle.

### Reconstitution

The lyophilized DP was reconstituted with Milli-Q filtered water (MQ) in Round 1, with either MQ water or TNS buffer (12.5 mM Tris, 12.5 mM NaCl, and 2.25% sucrose at pH 7.5) in Round 2, and with RNase-Free water in Round 3. The volume of reconstitution was 100 μL/well across all three rounds.

### Analytical Methods

Both pre-lyophilization and lyophilized samples were analyzed by the following analytical methods shown in Table 15.

Visual Inspection: Visual inspection was performed under a white light source (13W fluorescent tube) against black and white backgrounds. Digital photographs were acquired of all formulations at pre-lyophilization samples, lyophilized samples, and reconstituted samples.

TABLE 15
Analytical Methods Excipient Screening Study

| | Round 1 | | Round 2 | | Round 3 | |
|---|---|---|---|---|---|---|
| Analytical | | Lyophilized & | | Lyophilized & | | Lyophilized & |
| Methods | Pre-Lyo | Reconstituted | Pre-Lyo | Reconstituted | Pre-Lyo | Reconstituted |
| Visual | X | X | X | X | X | X |
| DLS | X | X | X | X | X | X |
| RiboGreen | X | X | X | X | X | X |

Visual Inspection: Visual inspection was performed under a white light source (13W fluorescent tube) against black and white backgrounds. Digital photographs were acquired of all formulations at pre-lyophilization samples, lyophilized samples, and reconstituted samples.

Dynamic Light Scattering (DLS): Changes in light intensity (scattered as particles diffuse by Brownian motion) were measured, and information about particle population, size, and size distribution were collected using the DLS DynaPro Plate Reader II. The standard was prepared by adding a drop of 3000 Series Nanosphere™ Size Standards (100 nm, Thermo Scientific, Cat #3100A) to a 1 mL aliquot of 1×PBS. For all sample sets, 100 nm Nanosphere size standard results met system suitability criteria of cumulants mean particle diameter (d. nm) of 100 nm±3 nm and the average polydispersity index (PDI)<0.1, as shown in Table A1. Samples were diluted to 0.1 mg/mL by diluting 10 μL of 0.25 mg/mL formulation in 240 μL of 1×PBS (pH 7.4). The DP control samples were diluted to 0.01 mg/mL by diluting 10 μL of 1 mg/mL DP control in 1 mL of 1×PBS. Both Nanosphere size standard and diluted samples were then degassed at 70 torr for 30 minutes to reduce the presence of air bubbles. After degassing, 25 μL/well of each sample was loaded into a 384-well plate in triplicate per sample. The plate was then centrifuged for 120 seconds at 2,000 rpm using a 5810 Eppendorf centrifuge. The plate reader was set to 25° C. and 10 acquisitions per sample at a rate of 5 seconds per acquisition, setting the total run time of 50 seconds/well. Particle sizes were analyzed in 1≤x≤10000 nm radii range. Particle radius distribution was categorized into Peak 1 (1-10 nm), Peak 2 (10-100 nm), Peak 3 (100-1000 nm), and Peak 4 (1000-5000 nm). All reported data is a cumulative average of these 10 acquisitions.

### RiboGreen Analysis (RNA Quantification)

RNA standard (100 μg/mL in Tris-EDTA (TE) buffer from Quant-iT™ RiboGreen™ RNA Assay Kit) was diluted to 10 μg/mL with 1×TE buffer. The 10 μg/mL RNA standard stock was then diluted to 5, 4, 3.2, 2.4, 1.6, 0.8, and 0.4 μg/mL stock solutions with 1×TE buffer. The standard stock solutions were then diluted 1:1 with either 1×TE or 2% Triton X-100. During each round samples were prepared by diluting to ˜50 μg/mL with 1×TE. Then, for Free RNA samples, an additional 1:10 dilution with TE buffer was performed to achieve 5 μg/mL; samples were not vortexed. For Total RNA samples, a 1:50 dilution was performed with 1% Triton X-100 to achieve 1 μg/mL. Each Total RNA sample was then vortexed for approximately 10 seconds for complete disruption. A RiboGreen working solution was prepared via 1:200 dilution of RiboGreen stock with 1×TE buffer. Following RNA standard and DP sample preparation, 100 μL of each sample was mixed with 100 μL of RiboGreen working solution and incubated in darkness at ambient temperature for 10 minutes. Two replicates were performed for RNA standards and three replicates were performed for DP samples. All plates were read on a fluorescence plate reader with excitation at 485 nm and emission at 530 nm. For all plates, standard curve R² values met the acceptance criteria of >0.975, and % RSD of the two standard replicates met the acceptance criteria of ≤20%. See for example, Tables A19-A26 below.

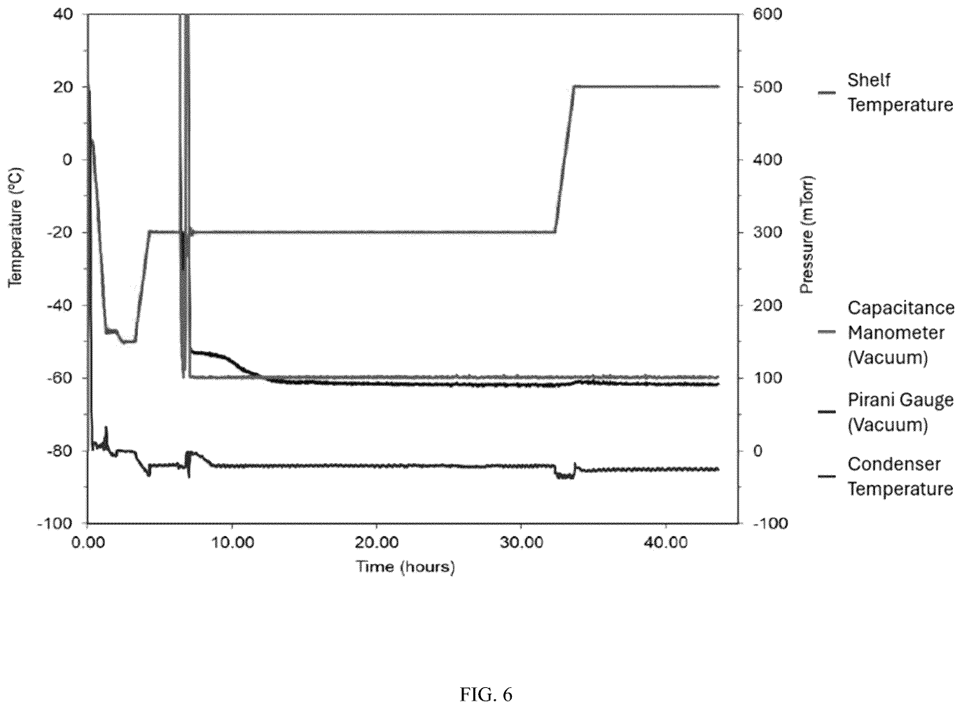

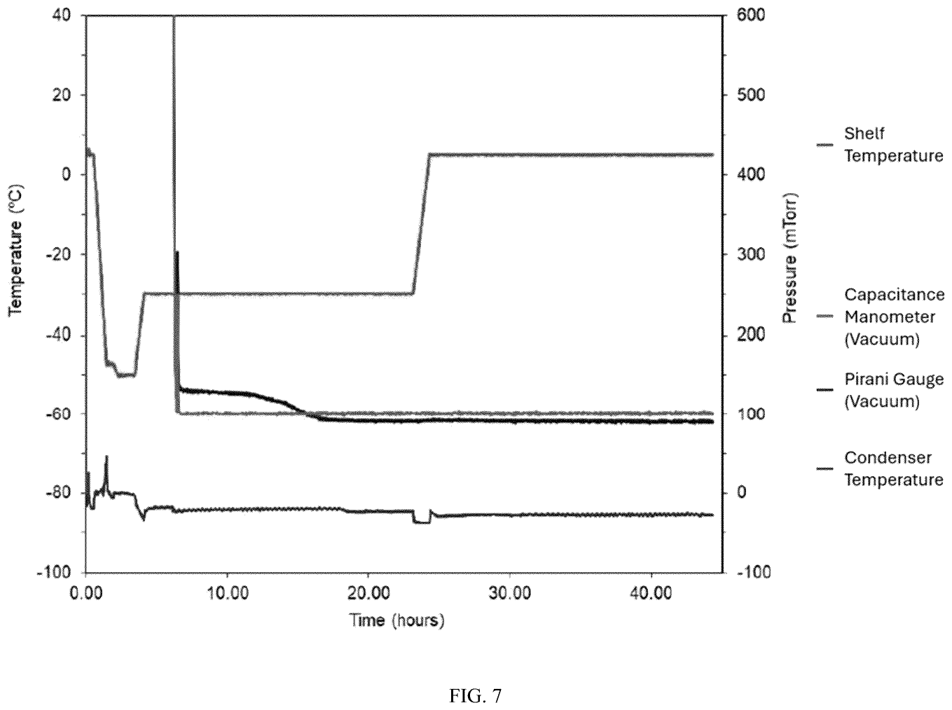

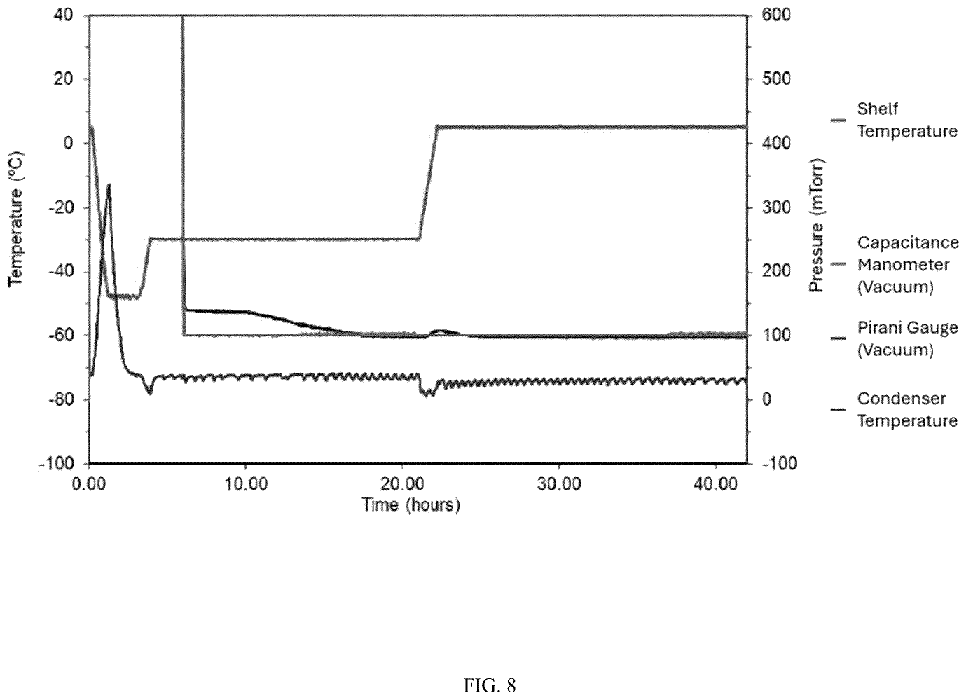

The lyophilization cycle profiles for each round of screening are shown in FIG. 6 through FIG. 8. For Round 1, primary drying at −20° C. took ˜12 hrs to reach a differential of <3 mT of Pirani Gauge/Capacitance Manometer (PVG/CM), indicating the completion of the primary drying process (FIG. 6). For Round 2 and Round 3, primary drying at −30° C. took 15-16 hours to reach a differential of <3 mT of PVG/CM (FIG. 7 and FIG. 8). The secondary drying was completed at 20° C. for Round 1 and at 5° C. for Rounds 1 and 3.

### Visual Observation

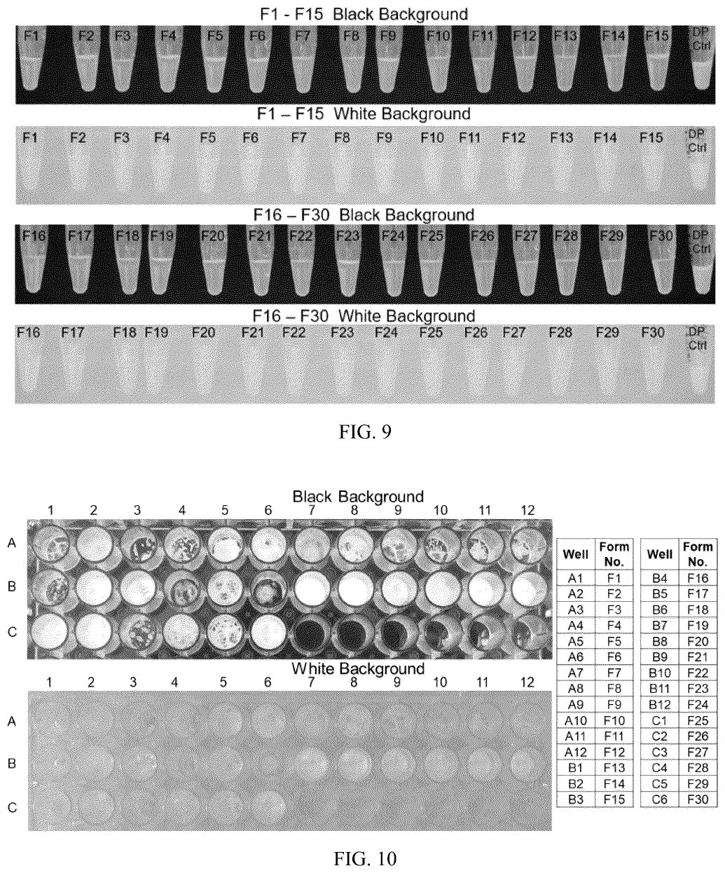

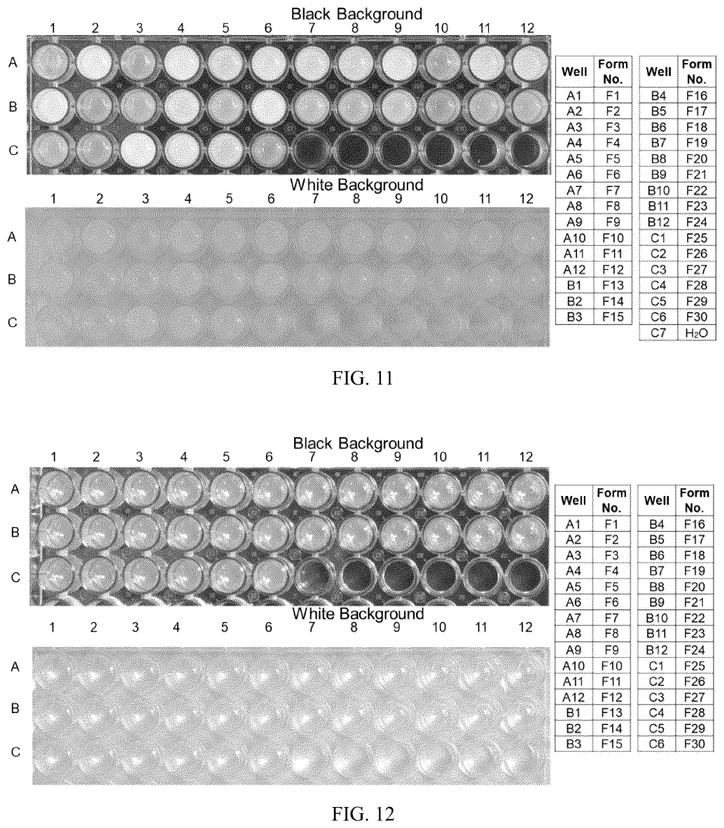

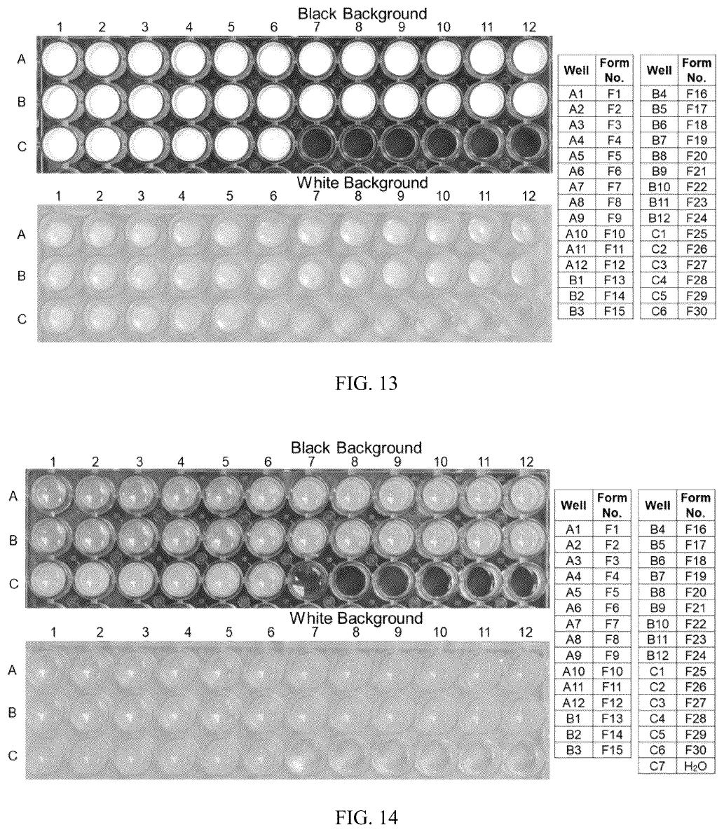

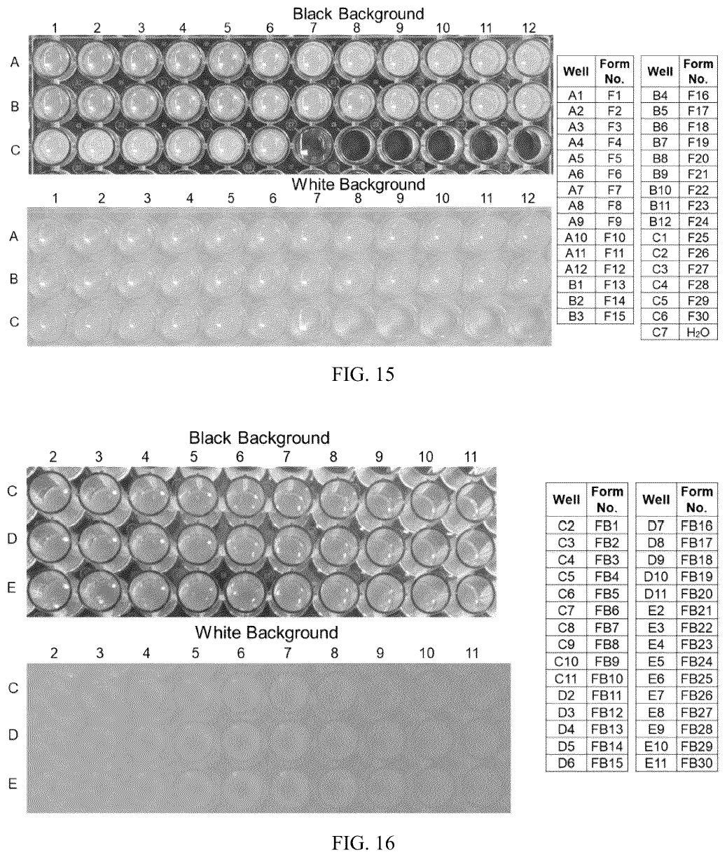

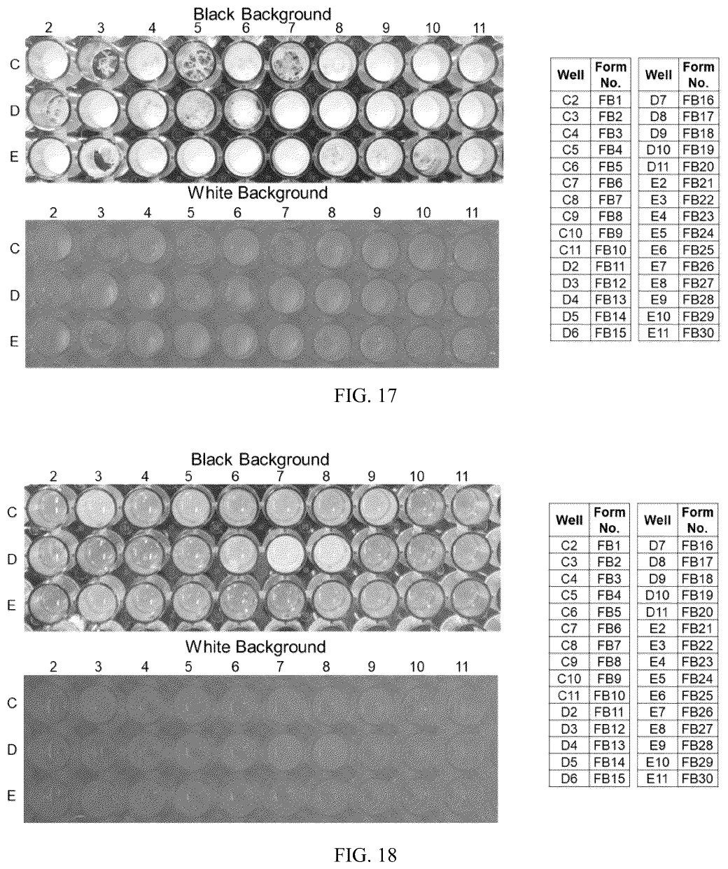

All formulations of cargo molecule were visually inspected before lyophilization (pre-lyo), after lyophilization (post-lyo), and after reconstitution for clarity, coloration, and visible precipitation in liquid samples, or for cake appearance in lyophilized samples. Photographs of the formulations in each plate were taken (FIG. 9 through FIG. 18). In all rounds of screening, pre-lyophilization samples (pre-lyo) at 0.25 mg/mL showed comparable visual appearances to each other, with slightly less opalescence than the DP (5° C.) control at 1 mg/mL.

After lyophilization, in Round 1, formulations containing 9% sucrose or iodixanol displayed elegant cakes with no or few minor bubbles. All the other formulations showed signs of collapse or displayed many bubbles. In Round 2, all 30 formulations contained 9% sucrose or iodixanol and displayed elegant cakes with no or few minor bubbles. In Round 3, formulations containing 9% sucrose, 50 mM NaCl, and 50 mM Tris-HCl displayed elegant cakes with a few minor bubbles. Increasing concentrations of NaCl (150 mM and 300 mM) or Tris-HCl (100 mM and 200 mM) resulted in highly porous or collapsed cakes. The Positive Control Buffer formulation displayed a porous lyophilized cake that was inflated from the well.

After reconstitution with water or 12.5 mM, Tris buffer containing 12.5 mM NaCl and 2.25% sucrose, pH 7.5 (TNS buffer), the various visual turbidities of the samples did not correlate to the lyophilized cake formations. In Round 1, the reconstituted formulations containing hydrophobic salts presented as white turbid liquids with undissolved precipitates, except for F17. Round 1 F17 formulation contained arginine with potassium sorbate and the reconstituted sample displayed a comparable opalescence level to its pre-lyophilization sample. Formulations containing 9% sugar (sucrose or iodixanol) and amino acids (arginine or proline) showed comparable opalescence levels to their pre-lyophilization samples. All other formulations that contained lower or higher sugar concentrations than 9% appeared to be more opalescent and turbid than their pre-lyophilization controls. In Round 2, samples were reconstituted with either filtered MQ water or TNS buffer. The reconstituted formulations containing 9% sucrose, with PVA10 or without P188, F1-F5, and F13-F17, showed comparable opalescence to their pre-lyophilization samples. All other formulations appeared to be more opalescent than their pre-lyophilization controls. The formulations were also less opalescent after reconstitution with TNS buffer compared to samples reconstituted with MQ water. In Round 3, all formulations reconstituted with RNase-free water were dissolved easily but increased in opalescence compared to their pre-lyophilization controls. Again, turbidity of the reconstituted formulations did not correlate with lyo-cake formation. For example, formulations containing Dextran 40, F16 and F17, were the most opalescent, but their lyophilized formulations displayed ideal cake formation.

### Dynamic Light Scattering (DLS) Analysis

Pre-lyophilization and reconstituted post-lyophilization of test sample were analyzed by dynamic light scattering (DLS) to determine particle size distribution. Upon meeting the system suitability (Table A1), DLS results for each round of screening are shown in Tables A2-A8 and Tables 16 and 17.

TABLE A1
Dynamic Light Scattering, System Suitability Criteria

| | Round 1 | | Round 2 | | Round 3 | |
|---|---|---|---|---|---|---|
| | Measured | | Measured | | Measured | |
| | Values | Passing | Values | Passing | Values | Passing |
| Criteria | (n = 3) | Values | (n = 3) | Values | (n = 3) | Values |
| d. nm | | 100 ± 3 nm | | 100 ± 3 nm | | 100 ± 3 nm |
| (100 nm | 97.5- | | 99.1- | | 101.6- | |
| Standard)* | 99.7 nm | | 100.9 nm | | 102.5 nm | |
| PDI | 0.03 | Average | 0.04 | Average | 0.03 | Average |
| | | measurement | | measurement | | measurement |
| | | must be less | | must be less | | must be less |
| | | than 0.1 | | than 0.1 | | than 0.1 |

TABLE A2
Round 1, Pre-Lyo, DLS

| | | Average | | Main Peak | |
|---|---|---|---|---|---|
| | | d · nm | | | % |
| Form No. | Form Code | (n = 3) | PDI | d · nm | Intensity |
| 100 nm | | 98.7 | 0.04 | 103.1 | 99.9-100.0 |
| DP Control | | 69.8 | 0.14 | 76.6 | 99.4-99.9 |
| F1 | H-VBN | 72.9 | 0.15 | 81.2 | 99.8-99.9 |
| F2 | H-VKN | 76.2 | 0.13 | 85.7 | 99.6-99.8 |
| F3 | H-FBN | 73.0 | 0.15 | 81.6 | 96.2-99.8 |
| F4 | H-FKN | 75.5 | 0.14 | 83.9 | 99.1-99.9 |
| F5 | T-VKN | 76.1 | 0.19 | 88.5 | 95.7-100.0 |
| F6 | T-VBN | 77.4 | 0.19 | 85.9 | 99.7-99.9 |
| F7 | T-FKN | 75.2 | 0.16 | 83.8 | 97.8-99.8 |
| F8 | T-FBN | 75.3 | 0.20 | 85.3 | 99.3-99.9 |
| F9 | H-VK | 76.2 | 0.14 | 88.7 | 99.9-99.9 |
| F10 | H-FB | 74.8 | 0.18 | 84.3 | 95.3-99.8 |
| F11 | T-VB | 78.3 | 0.22 | 93.6 | 99.0-100.0 |
| F12 | T-FK | 74.6 | 0.17 | 86.2 | 96.5-99.2 |
| F13 | T-F2SN | 74.5 | 0.17 | 82.2 | 97.3-99.9 |
| F14 | T-F9SN | 76.0 | 0.16 | 81.9 | 99.8-100.0 |
| F15 | T-V9SN | 74.9 | 0.19 | 85.0 | 99.8-99.9 |
| F16 | T-FRN | 74.9 | 0.16 | 83.4 | 99.4-99.9 |
| F17 | T-FKRN | 74.3 | 0.16 | 85.3 | 99.0-99.9 |
| F18 | T-FBPN | 75.4 | 0.18 | 87.1 | 99.6-99.9 |
| F19 | H-VPI | 71.7 | 0.15 | 81.0 | 98.0-99.9 |
| F20 | H-VRI | 72.0 | 0.18 | 82.0 | 99.9-99.9 |
| F21 | H-FP9S | 72.5 | 0.16 | 78.8 | 95.2-99.8 |
| F22 | H-FR9S | 72.1 | 0.17 | 76.1 | 99.0-100.0 |
| F23 | T-VPI | 74.4 | 0.17 | 83.8 | 99.6-99.9 |
| F24 | T-VRI | 74.4 | 0.17 | 81.0 | 98.7-99.8 |
| F25 | T-FP9S | 76.2 | 0.16 | 84.8 | 96.3-99.9 |
| F26 | T-FR9S | 74.8 | 0.16 | 81.2 | 98.4-99.9 |
| F27 | T-FP2SN | 74.1 | 0.17 | 83.6 | 92.4-99.9 |
| F28 | T-FP20SN | 76.1 | 0.16 | 80.5 | 94.9-99.8 |
| F29 | T-FR2SN | 74.8 | 0.18 | 86.4 | 98.4-99.9 |
| F30 | T-FR9SN | 75.5 | 0.19 | 89.7 | 99.9-99.9 |

TABLE A3
Round 1, Post-Lyo, DLS

| | | Average | | Main Peak | |
|---|---|---|---|---|---|
| | | d · nm | | | % |
| Form No. | Form Code | (n = 3) | PDI | d · nm | Intensity |
| 100 nm | | 98.8 | 0.03 | 101.5 | 99.8-99.9 |
| F1 | H-VBN | 188.6 | 0.45 | 182.5 | 94.3-99.3 |
| F2 | H-VKN | 215.5 | 0.17 | 218.0 | 97.2-99.0 |
| F3 | H-FBN | 171.7 | 0.35 | 175.1 | 92.0-95.8 |
| F4 | H-FKN | 244.2 | 0.39 | 276.8 | 95.6-99.4 |
| F5 | T-VKN | 146.1 | 0.44 | 138.4 | 82.4-91.0 |
| F6 | T-VBN | 228.9 | 0.41 | 244.4 | 87.6-100.0 |
| F7 | T-FKN | 155.6 | Multi | 166.1 | 88.7-99.8 |
| F8 | T-FBN | 213.4 | 0.48 | 217.5 | 90.9-94.0 |
| F9 | H-VK | 218.4 | 0.21 | 229.5 | 98.4-99.8 |
| F10 | H-FB | 179.8 | 0.31 | 173.1 | 96.9-99.9 |
| F11 | T-VB | 268.8 | Multi | 286.3 | 91.0-97.5 |
| F12 | T-FK | 173.5 | Multi | 191.1 | 98.6-99.9 |
| F13 | T-F2SN | 199.5 | 0.16 | 210.0 | 95.7-99.4 |
| F14 | T-F9SN | 96.2 | 0.2 | 118.5 | 99.8-100.0 |
| F15 | T-V9SN | 103.6 | 0.19 | 118.2 | 94.9-99.0 |
| F16 | T-FRN | 151.8 | 0.33 | 167.9 | 87.1-97.2 |
| F17 | T-FKRN | 112.1 | 0.39 | 134.7 | 83.3-99.4 |
| F18 | T-FBPN | 208.7 | 0.24 | 222.5 | 96.4-99.4 |
| F19 | H-VPI | 115.3 | 0.26 | 114.1 | 94.3-100.0 |
| F20 | H-VRI | 122.2 | Multi | 140.9 | 55.9-97.4 |
| F21 | H-FP9S | 131.3 | 0.2 | 132.7 | 97.3-100.0 |
| F22 | H-FR9S | 96.0 | 0.32 | 99.6 | 96.7-99.9 |
| F23 | T-VPI | 112.6 | Multi | 142.1 | 60.5-99.9 |
| F24 | T-VRI | 128.8 | Multi | 168.3 | 93.6-100.0 |
| F25 | T-FP9S | 111.0 | 0.24 | 137.6 | 96.4-100.0 |
| F26 | T-FR9S | 95.5 | 0.26 | 106.7 | 94.0-99.9 |
| F27 | T-FP2SN | 223.2 | 0.38 | 237.9 | 88.2-99.4 |
| F28 | T-FP20SN | 200.6 | 0.21 | 235.1 | 99.1-100.0 |
| F29 | T-FR2SN | 132.4 | 0.35 | 133.0 | 93.8-99.9 |
| F30 | T-FR9SN | 91.9 | 0.27 | 105.4 | 93.8-99.6 |

TABLE A4
Round 2, Pre-Lyo, DLS

| | Average | | Main Peak | |
|---|---|---|---|---|
| | d · nm | | | % |
| Sample | (n = 3) | PDI | d · nm | Intensity |
| 100 nm Standard | 99.5 | 0.03 | 103.2 | 99.9-100.0 |
| DP (5° C.) Control | 85.4 | 0.16 | 84.0 | 99.4-99.6 |
| F1 | 87.3 | 0.15 | 101.8 | 97.4-99.8 |
| F2 | 87.6 | 0.16 | 101.4 | 98.4-98.5 |
| F3 | 88.4 | 0.14 | 101.2 | 99.6-99.8 |
| F4 | 87.9 | 0.15 | 103.8 | 99.9-100.0 |
| F5 | 87.7 | 0.15 | 101.2 | 98.1-99.4 |
| F6 | 88.1 | 0.15 | 101.5 | 98.5-99.9 |
| F7 | 86.5 | 0.16 | 101.1 | 99.1-99.7 |
| F8 | 87.5 | 0.16 | 100.4 | 99.5-99.7 |
| F9 | 87.5 | 0.18 | 104.9 | 94.3-99.9 |
| F10 | 87.1 | 0.16 | 104.1 | 99.4-99.9 |
| F11 | 87.6 | 0.17 | 102.6 | 94.4-98.1 |
| F12 | 88.1 | 0.17 | 102.5 | 99.0-99.6 |
| F13 | 87.3 | 0.18 | 104.2 | 98.0-100.0 |
| F14 | 86.8 | 0.15 | 101.2 | 98.1-99.8 |
| F15 | 86.3 | 0.17 | 104.5 | 99.8-99.9 |
| F16 | 86.8 | 0.15 | 96.6 | 97.5-99.6 |
| F17 | 86.5 | 0.16 | 101.6 | 97.3-99.4 |
| F18 | 85.9 | 0.2 | 103.2 | 93.9-99.9 |
| F19 | 85.7 | 0.18 | 103.5 | 95.6-99.9 |
| F20 | 85.7 | 0.19 | 103.6 | 98.9-99.8 |
| F21 | 86.7 | 0.16 | 96.5 | 95.1-99.4 |
| F22 | 86.9 | 0.17 | 103.7 | 99.4-100.0 |
| F23 | 86.3 | 0.17 | 101.9 | 98.1-99.9 |
| F24 | 86.4 | 0.16 | 101.3 | 98.2-99.9 |
| F25 | 85.5 | 0.18 | 99.2 | 94.9-99.9 |
| F26 | 85.6 | 0.17 | 100.2 | 98.2-98.6 |
| F27 | 86 | 0.16 | 101.6 | 99.8-99.9 |
| F28 | 86.4 | 0.17 | 103.3 | 99.6-100.0 |
| F29 | 86.2 | 0.15 | 100.8 | 94.6-99.7 |
| F30 | 87.3 | 0.16 | 99.4 | 93.8-99.8 |

TABLE A5
Round 2, Reconstituted with Milli-Q water, DLS

| | |
|---|---|
| Average | Main Peak |

| | d · nm | | | % |
|---|---|---|---|---|
| Sample | (n = 3) | PDI | d · nm | Intensity |
| 100 nm Standard | 100.2 | 0.03 | 103.8 | 99.8-100.0 |
| F1 | 93.0 | 0.18 | 113.4 | 97.0-99.9 |
| F2 | 93.4 | 0.2 | 110.6 | 95.6-99.6 |
| F3 | 92.4 | 0.17 | 110.0 | 95.5-99.9 |
| F4 | 97.0 | 0.18 | 113.4 | 98.8-99.8 |
| F5 | 93.4 | 0.18 | 110.7 | 99.7-99.9 |
| F6 | 111.9 | 0.16 | 129.4 | 99.3-99.9 |
| F7 | 125.2 | 0.49 | 156.2 | 99.9-99.9 |
| F8 | 115.3 | 0.38 | 131.9 | 91.9-99.9 |
| F9 | 124.7 | 0.49 | 161.3 | 97.3-99.9 |
| F10 | 124.5 | 0.41 | 149.5 | 99.7-99.9 |
| F11 | 125.5 | 0.47 | 154.0 | 96.2-100.0 |
| F12 | 136.0 | 0.42 | 152.9 | 97.2-100.0 |
| F13 | 90.9 | 0.19 | 111.5 | 99.6-99.9 |
| F14 | 93.7 | 0.21 | 117.4 | 99.1-100.0 |
| F15 | 92.2 | 0.18 | 110.6 | 99.4-99.9 |
| F16 | 94.4 | 0.2 | 112.1 | 95.6-99.6 |
| F17 | 92.3 | 0.18 | 110.8 | 98.8-99.9 |
| F18 | 103.7 | 0.18 | 118.5 | 93.8-99.7 |
| F19 | 110.9 | 0.38 | 132.0 | 91.9-100.0 |
| F20 | 106.6 | 0.28 | 123.2 | 90.8-98.5 |
| F21 | 119.2 | 0.49 | 166.5 | 99.4-99.9 |
| F22 | 111.9 | 0.39 | 140.4 | 95.3-99.9 |
| F23 | 114.5 | 0.42 | 134.3 | 97.8-100.0 |
| F24 | 126.5 | 0.4 | 151.2 | 99.7-99.9 |
| F25 | 120.7 | 0.48 | 151.2 | 99.8-100.0 |
| F26 | 136.3 | 0.48 | 167.8 | 94.1-99.7 |
| F27 | 127.8 | 0.43 | 142.0 | 90.4-100.0 |
| F28 | 116.8 | 0.34 | 133.4 | 92.7-99.9 |
| F29 | 126.4 | 0.45 | 134.6 | 93.6-99.4 |
| F30 | 110.1 | 0.2 | 127.1 | 98.7-100.0 |

TABLE A6
Round 2, Reconstituted with TNS buffer, DLS

| | |
|---|---|
| Average | Main Peak |

| | d · nm | | | % |
|---|---|---|---|---|
| Sample | (n = 3) | PDI | d · nm | Intensity |
| 100 nm Standard | 99.5 | 0.04 | 104.3 | 99.9-100.0 |
| F1 | 91.7 | 0.18 | 100.8 | 97.3-99.7 |
| F2 | 92.7 | 0.18 | 92.6 | 97.9-99.5 |
| F3 | 90.1 | 0.18 | 84.4 | 99.5-99.8 |
| F4 | 99.3 | 0.18 | 89.1 | 99.3-100.0 |
| F5 | 94.0 | 0.18 | 89.5 | 99.6-100.0 |
| F6 | 109.8 | 0.16 | 115.5 | 98.2-100.0 |
| F7 | 112.7 | 0.38 | 123.1 | 97.6-99.9 |
| F8 | 112.7 | 0.25 | 96.5 | 98.5-100.0 |
| F9 | 123.9 | 0.41 | 133.8 | 90.0-99.9 |
| F10 | 118.5 | 0.34 | 122.1 | 96.8-100.0 |
| F11 | 117.9 | 0.41 | 130.2 | 91.9-99.3 |
| F12 | 134.2 | 0.34 | 109.6 | 98.4-99.8 |
| F13 | 90.0 | 0.19 | 90.3 | 99.5-99.9 |
| F14 | 92.4 | 0.18 | 96.3 | 99.2-100.0 |
| F15 | 91.8 | 0.17 | 101.2 | 99.0-100.0 |
| F16 | 95.9 | 0.2 | 107.8 | 98.8-99.6 |
| F17 | 94.4 | 0.18 | 102.6 | 94.1-99.8 |
| F18 | 106.0 | 0.18 | 114.4 | 96.4-98.9 |
| F19 | 101.4 | 0.3 | 106.4 | 90.8-99.8 |
| F20 | 104.2 | 0.28 | 110.6 | 98.8-100.0 |
| F21 | 109.2 | 0.43 | 121.4 | 99.6-99.9 |
| F22 | 104.3 | 0.28 | 102.4 | 90.5-99.0 |
| F23 | 108.1 | 0.38 | 127.1 | 98.0-99.9 |
| F24 | 122.7 | 0.39 | 142.0 | 99.5-99.9 |
| F25 | 111.8 | 0.38 | 121.3 | 95.0-99.9 |
| F26 | 120.1 | 0.39 | 122.4 | 93.5-99.9 |
| F27 | 120.5 | 0.44 | 131.0 | 92.5-99.9 |
| F28 | 113.3 | 0.37 | 117.1 | 91.4-99.9 |
| F29 | 127.5 | 0.29 | 92.2 | 100.0-100.0 |
| F30 | 105.0 | 0.19 | 116.8 | 95.5-99.9 |

TABLE A7
Round 3, Pre-Lyo, DLS

| | Average | | Main Peak | |
|---|---|---|---|---|
| | d · nm | | | % |
| Sample | (n = 3) | PDI | d · nm | Intensity |
| 100 nm Standard | 100.1 | 0.03 | 102.1 | 99.7-100.0 |
| DP (5° C.) Control | 87.4 | 0.2 | 96.1 | 95.9-99.6 |
| FB1 | 89.1 | 0.21 | 100.0 | 99.3-99.8 |
| FB2 | 85.9 | 0.18 | 90.5 | 99.6-99.9 |
| FB3 | 86.2 | 0.19 | 102.7 | 99.1-99.9 |
| FB4 | 89.9 | 0.25 | 97.3 | 99.9-99.9 |
| FB5 | 86.0 | 0.19 | 98.9 | 99.7-99.9 |
| FB6 | 85.5 | 0.17 | 98.4 | 98.9-99.9 |
| FB7 | 86.2 | 0.18 | 97.3 | 97.1-99.9 |
| FB8 | 89.2 | 0.22 | 94.2 | 97.6-99.8 |
| FB9 | 86.2 | 0.2 | 100.6 | 99.7-99.9 |
| FB10 | 90.3 | 0.2 | 98.4 | 97.5-99.9 |
| FB11 | 88.1 | 0.2 | 101.8 | 99.3-100.0 |
| FB12 | 86.3 | 0.19 | 102.0 | 99.5-99.9 |
| FB13 | 87.7 | 0.2 | 99.8 | 99.7-99.9 |
| FB14 | 85.9 | 0.19 | 100.0 | 98.5-99.9 |
| FB15 | 88.1 | 0.19 | 101.7 | 98.3-99.9 |
| FB16 | 86.7 | 0.2 | 97.1 | 99.6-99.9 |
| FB17 | 88.3 | 0.19 | 108.6 | 99.8-100.0 |
| FB18 | 85.8 | 0.18 | 94.2 | 94.0-99.3 |
| FB19 | 87.0 | 0.22 | 103.1 | 99.5-99.9 |
| FB20 | 85.3 | 0.17 | 94.5 | 99.7-99.9 |
| FB21 | 84.8 | 0.19 | 99.4 | 99.6-99.9 |
| FB22 | 82.7 | 0.19 | 94.9 | 98.1-99.6 |
| FB23 | 85.2 | 0.18 | 96.4 | 99.8-99.9 |
| FB24 | 84.2 | 0.19 | 89.8 | 99.0-99.9 |
| FB25 | 85.3 | 0.18 | 98.4 | 98.1-100.0 |
| FB26 | 85.0 | 0.18 | 101.9 | 99.8-100.0 |
| FB27 | 87.9 | 0.18 | 103.8 | 99.5-99.8 |
| FB28 | 84.5 | 0.16 | 95.0 | 92.9-99.8 |
| FB29 | 85.9 | 0.18 | 93.7 | 98.8-99.9 |
| FB30 | 84.9 | 0.19 | 100.1 | 98.6-100.0 |

TABLE A8
Round 3, Reconstituted with RNase-free water, DLS

| | |
|---|---|
| Average | Main Peak |

| | d · nm | | | % |
|---|---|---|---|---|
| Sample | (n = 3) | PDI | d · nm | Intensity |
| 100 nm Standard | 100.1 | 0.03 | .1 | 99.7-100.0 |
| F1 | 108.8 | 0.26 | 129.3 | 98.4-99.9 |
| F2 | 176.1 | 0.19 | 217.3 | 90.8-99.6 |
| F3 | 116.9 | 0.24 | 139.8 | 99.6-100.0 |
| F4 | 139.1 | 0.14 | 158.5 | 98.2-100.0 |
| F5 | 123.8 | 0.29 | 135.4 | 97.5-99.9 |
| F6 | 134.0 | 0.29 | 139.8 | 94.1-99.9 |
| F7 | 118.8 | 0.30 | 131.6 | 98.8-99.9 |
| F8 | 145.3 | 0.44 | 160.2 | 80.4-99.9 |
| F9 | 95.2 | 0.22 | 116.6 | 99.9-100.0 |
| F10 | 105.8 | 0.22 | 115.1 | 95.4-99.8 |
| F11 | 116.8 | 0.13 | 128.8 | 97.3-99.5 |
| F12 | 103.2 | 0.22 | 124.9 | 99.6-99.9 |
| F13 | 103.5 | 0.21 | 113.1 | 99.1-99.9 |
| F14 | 113.4 | 0.17 | 124.4 | 89.9-98.3 |
| F15 | 134.4 | 0.16 | 155.7 | 98.4-99.9 |
| F16 | 208.5 | 0.29 | 203.6 | 97.2-99.6 |
| F17 | 249.0 | 0.46 | 271.3 | 94.4-99.5 |
| F18 | 111.6 | 0.26 | 112.1 | 98.6-99.9 |
| F19 | 105.0 | 0.27 | 111.3 | 93.5-99.9 |
| F20 | 119.8 | 0.25 | 141.2 | 99.5-99.9 |
| F21 | 97.7 | 0.24 | 120.2 | 99.8-99.9 |
| F22 | 189.5 | 0.22 | 223.8 | 97.4-99.7 |
| F23 | 127.3 | 0.27 | 161.1 | 99.4-99.9 |
| F24 | 121.7 | 0.24 | 154.8 | 99.7-99.9 |
| F25 | 95.2 | 0.23 | 108.8 | 98.5-99.8 |
| F26 | 93.9 | 0.19 | 108.1 | 88.2-99.5 |
| F27 | 127.2 | 0.25 | 155.7 | 99.0-99.9 |
| F28 | 124.9 | 0.26 | 152.0 | 98.1-100.0 |
| F29 | 111.5 | 0.14 | 123.9 | 99.7-99.9 |
| F30 | 99.3 | 0.23 | 111.4 | 96.9-100.0 |

All pre-lyophilization formulations showed comparable hydrodynamic diameters and main peak intensity percentages to DP control (Table 16). Following lyophilization, however, the reconstituted formulations showed increases in hydrodynamic diameters and polydispersity (PDI) compared to their pre-lyophilization controls (Table 17).

In Round 1, formulations containing 9% sucrose, formulations containing PVA10 with sucrose or iodixanol, and F17 showed smaller increases in hydrodynamic diameters. Among them, formulations containing 9% sucrose with either poloxamer 188, arginine, or salt showed smaller increases in hydrodynamic diameters compared to the other formulations. In Round 2, reconstitution with MQ or TNS (pH 7.5) following lyophilization yielded similar results. All formulations showed increases in hydrodynamic diameters compared to their pre-lyophilization controls. Formulations containing 9% sucrose, showed smaller increases in hydrodynamic diameters compared to those containing iodixanol. Formulations without P188 showed larger increases than their P188-containing counterparts. All formulations showed comparable main peak intensity percentages. In Round 3, all formulations were reconstituted with RNase-free water. All reconstituted formulations showed increases in hydrodynamic diameters, 46% to 150%, compared to those in their pre-lyophilization state. Formulations containing Dextran 40 showed the largest increases in hydrodynamic diameters. Formulations containing arginine and PVA10 showed smaller increases in hydrodynamic diameters. All formulation samples showed comparable main peak intensity percentages.

TABLE 16
Dynamic Light Scattering, Pre-lyophilization formulations

| | | Average | | Main Peak | |
|---|---|---|---|---|---|
| | | diameter | | Cumulative | Main Peak |
| Round | | (nm) | | Diameter | Intensity |
| No. | Formulation | (n = 3) | PDI | (nm) | (%) |
| Round 1 | DP Control | 69.80 | 0.14 | 76.60 | 99.4-99.9 |
| | Ave. | 74.80 | 0.17 | 84.08 | 95.2-100.0 |
| | Formulations | | | | |
| Round 2 | DP Control | 85.40 | 0.16 | 84.00 | 99.4-99.6 |
| | Ave. | 86.85 | 0.16 | 101.76 | 93.8-100.0 |
| | Formulations | | | | |
| Round 3 | DP Control | 87.10 | 0.20 | 96.10 | 95.9-99.6 |
| | Ave. | 86.47 | 0.19 | 98.48 | 92.9-100.0 |
| | Formulations | | | | |

TABLE 17
Dynamic Light Scattering Top Performing
Formulations of Each Round

| | | Cumulative | | Main Peak | Main |
|---|---|---|---|---|---|
| | | Average | | Cumulative | Peak |
| Round | Form. | Diameter | | Diameter | Intensity |
| No. | No. | (nm) | PDI | (nm) | (%) |
| Round 1 | Lyo: Milli-Q filtered water Reconstituted | | | | |
| | F22 | 96 | 0.32 | 99.6 | 96.7-99.9 |
| | F30 | 91.9 | 0.27 | 105.4 | 93.8-99.6 |
| | F26 | 95.5 | 0.26 | 106.7 | 94.0-99.9 |
| | F19 | 115.3 | 0.26 | 114.1 | 94.3-100.0 |
| | F15 | 103.6 | 0.19 | 118.2 | 94.9-99.0 |
| | F14 | 96.2 | 0.2 | 118.5 | 99.8-100.0 |
| Round 2 | Lyo: Milli-Q filtered water Reconstituted | | | | |
| | F3 | 92.4 | 0.17 | 110 | 95.5-99.9 |
| | F2 | 93.4 | 0.2 | 110.6 | 95.6-99.6 |
| | F15 | 92.2 | 0.18 | 110.6 | 99.4-99.9 |
| | F5 | 93.4 | 0.18 | 110.7 | 99.7-99.9 |
| | F17 | 92.3 | 0.18 | 110.8 | 98.8-99.9 |
| | F13 | 90.9 | 0.19 | 111.5 | 99.6-99.9 |
| | F16 | 94.4 | 0.2 | 112.1 | 95.6-99.6 |
| | F1 | 93 | 0.18 | 113.4 | 97.0-99.9 |
| | F4 | 97 | 0.18 | 113.4 | 98.8-99.8 |
| | Lyo: TNS, pH 7.5, Reconstituted | | | | |
| | F1 | 91.7 | 0.18 | 84.4 | 97.3-99.7 |
| | F2 | 92.7 | 0.18 | 89.1 | 97.9-99.5 |
| | F3 | 90.1 | 0.18 | 89.5 | 99.5-99.8 |
| | F4 | 99.3 | 0.18 | 90.3 | 99.3-100.0 |
| | F5 | 94 | 0.18 | 92.2 | 99.6-100.0 |
| | F6 | 109.8 | 0.16 | 92.6 | 98.2-100.0 |
| Round 3 | Lyo: RNase-Free Water Reconstituted | | | | |
| | F26 | 93.9 | 0.19 | 108.1 | 88.2-99.5 |
| | F25 | 95.2 | 0.23 | 108.8 | 98.5-99.8 |
| | F19 | 105 | 0.27 | 111.3 | 93.5-99.9 |
| | F30 | 99.3 | 0.23 | 111.4 | 96.9-100.0 |
| | F18 | 111.6 | 0.26 | 112.1 | 98.6-99.9 |
| | F13 | 103.5 | 0.21 | 113.1 | 99.1-99.9 |
| | F10 | 105.8 | 0.22 | 115.1 | 95.4-99.8 |

* n = 3
** Ranked by Main Peak Cumulative Diameter, from smallest to largest

### RiboGreen Analysis

In each round of this study, pre-lyophilization and post-lyophilization formulations of test sample were analyzed with RiboGreen RNA-binding fluorescence assay to quantify RNA concentrations. mRNA concentrations of the formulations before and after treatment with TritonX-100 (TX100) were used to calculate percentages of encapsulated mRNA. All assay plates met system suitability; results can be found in Tables A17-A26. Complete RiboGreen assay results for each round of experiments are shown in Tables A8-A16.

In each round of the study, pre-lyophilized samples of all formulations showed comparable percent encapsulation to the DP control (Table 18). Lyophilization and reconstitution with MQ water in Round 1 resulted in decreased encapsulation, with losses ranging from −9.4% to −74.4%, compared to pre-lyophilization samples. Following lyophilization, PVA-containing formulations with iodixanol or sucrose showed higher encapsulation percentages compared to the other formulations, as is seen in Table 19. The addition of arginine or NaCl also resulted in some retention of encapsulation during lyophilization.

In Round 2, lyophilization and reconstitution with MQ water or TNS buffer led to decreased encapsulation compared to pre-lyophilization samples. Losses for reconstitution with MQ water ranged from −7.1% to −61.1%, and reconstitution with TNS buffer ranged from −9.9% to −70.2%. However, reconstitution with MQ water yielded better encapsulation results than reconstitution with TNS buffer for all 30 formulations (+0.3% to +12.2%). Overall, reconstitution with MQ water is preferable compared to TNS buffer. Formulations containing iodixanol experienced smaller losses of encapsulated RNA after reconstitution, for both reconstitutions with MQ water and TNS buffer. The addition of NaCl continued to show benefits in preserving encapsulation through lyophilization. The top-performing formulations for Round 2 can be found in Table 19.

In Round 3, lyophilization and reconstitution with RNase-free water resulted in decreased percent encapsulation compared to pre-lyophilization samples. Losses ranged from −3.8% to −42.7%. Formulations containing PVA10 and arginine experienced the smallest losses of encapsulated RNA after lyophilization. High concentrations of Tris-HCl and NaCl also resulted in smaller losses of encapsulated RNA after lyophilization. Both EDTA and methionine appeared to have no significant effect in either direction, while formulations with Dextran 40 had the greatest loss of encapsulated RNA after lyophilization. Overall, the groups of F9, F25, and F26 and F13, F29, and F30 containing PVA10 and arginine experienced smaller losses of encapsulated RNA, as seen in Table 19.

TABLE 18
Pre- Lyophilization Formulations
of Each Round by RiboGreen assay

| | | Free | Total | |
|---|---|---|---|---|
| | | RNA | RNA | |
| Round | | Conc. | Conc. | % |
| No. | Formulation | (ug/mL) | (ug/mL) | Encap. |
| 1 | DP Control | 76.8 | 1208.9 | 93.6 |
| | Ave. Formulations | 16.9 | 241.4 | 93.0 |
| 2 | DP Control | 10.4 | 1145.8 | 99.1 |
| | Ave. Formulations | 5.3 | 273.5 | 98.1 |
| 3 | DP Control | 11.4 | 980.6 | 98.8 |
| | Ave. Formulations | 3.1 | 240.1 | 98.7 |

TABLE 19
Lyophilization Formulations of Each Round by RiboGreen assay

| | | Free RNA | Total RNA | |
|---|---|---|---|---|
| Round | Form. | Conc. | Conc. | % |
| No. | No. | (μg/mL) | (μg/mL) | Encapsulation |
| Round 1 | Lyo: Milli-Q Reconstituted | | | |
| | F15 | 37.8 | 241.1 | 84.3 |
| | F24 | 41.2 | 248.2 | 83.4 |
| | F20 | 39.1 | 224.7 | 82.6 |
| | F19 | 50.6 | 252.4 | 79.9 |
| | F23 | 54.4 | 266.9 | 79.6 |
| | F17 | 81.9 | 283.1 | 71.1 |
| Round 2 | Lyo: Milli-Q Reconstituted | | | |
| | F30 | 19.2 | 235.0 | 91.8 |
| | F29 | 21.4 | 229.5 | 90.7 |
| | F20 | 21.3 | 222.6 | 90.4 |
| | F24 | 24.0 | 230.6 | 89.6 |
| | F28 | 24.3 | 223.4 | 89.1 |
| | F19 | 25.6 | 228.8 | 88.8 |
| | F8 | 29.6 | 247.2 | 88.0 |
| | Lyo: TNS, pH 7.5, Reconstituted | | | |
| | F29 | 26.2 | 243.4 | 89.2 |
| | F16 | 27.4 | 217.2 | 87.4 |
| | F24 | 30.2 | 231.3 | 86.9 |
| | F20 | 30.4 | 227.0 | 86.6 |
| | F28 | 29.2 | 213.4 | 86.3 |
| | F19 | 33.2 | 232.5 | 85.7 |
| | F30 | 33.1 | 220.3 | 85.0 |
| Round 3 | Lyo: RNase-Free Water Reconstituted | | | |
| | F14 | 11.3 | 216.9 | 94.8 |
| | F29 | 10.3 | 191.3 | 94.6 |
| | F11 | 12.5 | 198.1 | 93.7 |
| | F13 | 15.5 | 214.1 | 92.7 |
| | F30 | 15.2 | 201.9 | 92.5 |
| | F10 | 18.1 | 221.7 | 91.8 |
| | F4 | 18.7 | 193.3 | 90.3 |
| | F12 | 21.6 | 210.6 | 89.7 |
| | F26 | 20.9 | 198.2 | 89.5 |
| | F15 | 23.9 | 224.5 | 89.4 |

TABLE A9
Round 1, Pre-Lyo, RiboGreen assay

| | | | Free RNA | Total RNA | |
|---|---|---|---|---|---|
| | Form | Form | Conc. | Conc. | |
| | No. | Code | (μg/mL) | (μg/mL) | % Encap. |
| DP Control | | | 76.8 | 1208.9 | 93.6 |
| | F1 | H-VBN | 15.1 | 228.6 | 93.4 |
| | F2 | H-VKN | 17.8 | 239.0 | 92.6 |
| | F3 | H-FBN | 15.8 | 231.1 | 93.2 |
| | F4 | H-FKN | 20.6 | 251.3 | 91.8 |
| | F5 | T-VKN | 18.6 | 247.0 | 92.5 |
| | F6 | T-VBN | 18.1 | 248.2 | 92.7 |
| | F7 | T-FKN | 14.9 | 239.0 | 93.8 |
| | F8 | T-FBN | 19.6 | 251.9 | 92.2 |
| | F9 | H-VK | 20.3 | 266.1 | 92.4 |
| | F10 | H-FB | 18.9 | 245.0 | 92.3 |
| | F11 | T-VB | 13.5 | 231.0 | 94.2 |
| | F12 | T-FK | 12.5 | 227.2 | 94.5 |
| | F13 | T-F2SN | 16.4 | 239.3 | 93.1 |
| | F14 | T-F9SN | 17.4 | 232.9 | 92.5 |
| | F15 | T-V9SN | 14.9 | 236.1 | 93.7 |
| | F16 | T-FRN | 14.2 | 234.8 | 93.9 |
| | F17 | T-FKRN | 20.7 | 262.0 | 92.1 |
| | F18 | T-FBPN | 19.3 | 240.7 | 92.0 |
| | F19 | H-VPI | 14.0 | 227.0 | 93.8 |
| | F20 | H-VRI | 12.9 | 223.2 | 94.2 |
| | F21 | H-FP9S | 16.9 | 235.1 | 92.8 |
| | F22 | H-FR9S | 17.8 | 232.0 | 92.3 |
| | F23 | T-VPI | 16.0 | 232.6 | 93.1 |
| | F24 | T-VRI | 14.8 | 232.0 | 93.6 |
| | F25 | T-FP9S | 18.8 | 260.1 | 92.8 |
| | F26 | T-FR9S | 15.7 | 247.5 | 93.6 |
| | F27 | T-FP2SN | 18.0 | 252.5 | 92.9 |
| | F28 | T-FP20SN | 17.5 | 247.5 | 92.9 |
| | F29 | T-FR2SN | 17.5 | 247.5 | 92.9 |
| | F30 | T-FR9SN | 18.9 | 254.4 | 92.6 |

TABLE A10
Round 1, Reconstituted with Milli-Q water, RiboGreen assay 1

| | | Free RNA | Total RNA | |
|---|---|---|---|---|
| Form | Form | Conc. | Conc. | |
| No. | Code | (μg/mL) | (μg/mL) | % Encap. |
| F1 | H-VBN | 191.9 | 248.2 | 22.7 |
| F2 | H-VKN | 185.6 | 258.5 | 28.2 |
| F3 | H-FBN | 187.0 | 245.8 | 23.9 |
| F4 | H-FKN | 162.3 | 204.7 | 20.7 |
| F5 | T-VKN | 164.8 | 209.0 | 21.1 |
| F6 | T-VBN | 166.9 | 250.6 | 33.4 |
| F7 | T-FKN | 172.5 | 245.8 | 29.8 |
| F8 | T-FBN | 172.2 | 253.0 | 31.9 |
| F9 | H-VK | 156.9 | 193.8 | 19.1 |
| F10 | H-FB | 165.7 | 221.1 | 25.0 |
| F11 | T-VB | 168.4 | 226.5 | 25.6 |
| F12 | T-FK | 146.6 | 183.5 | 20.1 |
| F13 | T-F2SN | 148.2 | 235.6 | 37.1 |
| F14 | T-F9SN | 118.1 | 246.5 | 52.1 |
| F15 | T-V9SN | 37.8 | 241.1 | 84.3 |
| F16 | T-FRN | 114.9 | 286.5 | 59.9 |
| F17 | T-FKRN | 81.9 | 283.1 | 71.1 |
| F18 | T-FBPN | 167.6 | 269.3 | 37.8 |
| F19 | H-VPI | 50.6 | 252.4 | 79.9 |
| F20 | H-VRI | 39.1 | 224.7 | 82.6 |
| F21 | H-FP9S | 145.7 | 254.2 | 42.7 |
| F22 | H-FR9S | 119.0 | 253.0 | 53.0 |
| F23 | T-VPI | 54.4 | 266.9 | 79.6 |
| F24 | T-VRI | 41.2 | 248.2 | 83.4 |
| F25 | T-FP9S | 140.1 | 281.2 | 50.2 |
| F26 | T-FR9S | 124.3 | 275.2 | 54.8 |
| F27 | T-FP2SN | 171.4 | 262.6 | 34.7 |
| F28 | T-FP20SN | 123.1 | 217.7 | 43.5 |
| F29 | T-FR2SN | 91.1 | 251.8 | 63.8 |
| F30 | T-FR9SN | 113.7 | 247.6 | 54.1 |

TABLE A11
RiboGreen, Pre-lyophilization Samples, Round 2

| | Free RNA | Total RNA | |
|---|---|---|---|
| | Conc. | Conc. | |
| Sample | (μg/mL) | (μg/mL) | % Encap. |
| DP (5° C.) | 10.4 | 1145.8 | 99.1 |
| Control | | | |
| F1 | 6.9 | 285.6 | 97.6 |
| F2 | 7.2 | 271.9 | 97.3 |
| F3 | 7.2 | 270.6 | 97.3 |
| F4 | 7.1 | 275.8 | 97.4 |
| F5 | 6.6 | 273.8 | 97.6 |
| F6 | 6.8 | 266.7 | 97.4 |
| F7 | 6.6 | 273.8 | 97.6 |
| F8 | 6.9 | 277.7 | 97.7 |
| F9 | 5.9 | 277.5 | 97.9 |
| F10 | 5.4 | 275.5 | 98.0 |
| F11 | 5.4 | 263.5 | 97.9 |
| F12 | 4.9 | 281.5 | 98.2 |
| F13 | 5.6 | 274.8 | 98.0 |
| F14 | 5.6 | 274.2 | 98.0 |
| F15 | 5.8 | 269.5 | 97.9 |
| F16 | 6.0 | 274.8 | 97.8 |
| F17 | 1.8 | 264.7 | 99.3 |
| F18 | 4.9 | 266.1 | 98.2 |
| F19 | 5.1 | 260.7 | 98.0 |
| F20 | 4.1 | 262.1 | 98.4 |
| F21 | 4.9 | 265.4 | 98.1 |
| F22 | 5.1 | 272.1 | 98.1 |
| F23 | 4.6 | 269.4 | 98.3 |
| F24 | 4.8 | 281.5 | 98.3 |
| F25 | 4.6 | 278.2 | 98.4 |
| F26 | 4.4 | 276.8 | 98.4 |
| F27 | 3.4 | 284.4 | 98.8 |
| F28 | 4.2 | 263.8 | 98.4 |
| F29 | 3.2 | 282.3 | 98.9 |
| F30 | 4.3 | 291.3 | 98.5 |

TABLE A12
RiboGreen, Lyo-milli-Q Water Reconstituted Samples, Round 2

| | Free RNA | Total RNA | |
|---|---|---|---|
| | Conc. | Conc. | |
| Sample | (μg/mL) | (μg/mL) | % Encap. |
| F1 | 127.6 | 232.9 | 45.2 |
| F2 | 144.9 | 239.1 | 39.4 |
| F3 | 147.1 | 230.5 | 36.2 |
| F4 | 37.3 | 224.5 | 83.4 |
| F5 | 140.7 | 244.9 | 42.6 |
| F6 | 62.5 | 243.1 | 74.3 |
| F7 | 36.9 | 246.7 | 85.0 |
| F8 | 29.6 | 247.2 | 88.0 |
| F9 | 52.6 | 242.1 | 78.3 |
| F10 | 46.7 | 247.4 | 81.1 |
| F11 | 69.0 | 240.3 | 71.3 |
| F12 | 32.2 | 228.9 | 85.9 |
| F13 | 114.2 | 231.9 | 50.7 |
| F14 | 125.2 | 240.3 | 47.9 |
| F15 | 127.2 | 229.5 | 44.6 |
| F16 | 28.6 | 233.7 | 87.8 |
| F17 | 123.3 | 241.8 | 49.0 |
| F18 | 46.2 | 217.7 | 78.8 |
| F19 | 25.6 | 228.8 | 88.8 |
| F20 | 21.3 | 222.6 | 90.4 |
| F21 | 35.4 | 232.5 | 84.8 |
| F22 | 30.6 | 229.4 | 86.7 |
| F23 | 39.8 | 233.1 | 82.9 |
| F24 | 24.0 | 230.6 | 89.6 |
| F25 | 68.0 | 241.7 | 71.9 |
| F26 | 59.1 | 232.6 | 74.6 |
| F27 | 37.8 | 238.1 | 84.1 |
| F28 | 24.3 | 223.4 | 89.1 |
| F29 | 21.4 | 229.5 | 90.7 |
| F30 | 19.2 | 235.0 | 91.8 |

TABLE A13
RiboGreen, Lyo-TNS Buffer, pH 7.5
Reconstituted Samples, Round 2

| | Free RNA | Total RNA | |
|---|---|---|---|
| | Conc. | Conc. | |
| Sample | (μg/mL) | (μg/mL) | % Encap. |
| F1 | 134.4 | 212.5 | 36.7 |
| F2 | 143.8 | 214.3 | 32.9 |
| F3 | 149.8 | 210.7 | 28.9 |
| F4 | 35.4 | 208.8 | 83.1 |
| F5 | 149.5 | 215.0 | 30.4 |
| F6 | 58.6 | 211.3 | 72.2 |
| F7 | 34.0 | 218.6 | 84.5 |
| F8 | 37.0 | 234.6 | 84.2 |
| F9 | 47.4 | 206.8 | 77.1 |
| F10 | 50.6 | 204.4 | 75.3 |
| F11 | 70.9 | 219.7 | 67.7 |
| F12 | 33.6 | 209.9 | 84.0 |
| F13 | 123.2 | 214.8 | 42.7 |
| F14 | 128.2 | 217.2 | 41.0 |
| F15 | 129.9 | 215.4 | 39.7 |
| F16 | 27.4 | 217.2 | 87.4 |
| F17 | 127.5 | 220.2 | 42.1 |
| F18 | 49.1 | 210.4 | 76.7 |
| F19 | 33.2 | 232.5 | 85.7 |
| F20 | 30.4 | 227.0 | 86.6 |
| F21 | 48.3 | 241.1 | 80.0 |
| F22 | 38.3 | 234.3 | 83.6 |
| F23 | 41.7 | 228.2 | 81.7 |
| F24 | 30.2 | 231.3 | 86.9 |
| F25 | 68.6 | 222.8 | 69.2 |
| F26 | 74.2 | 237.8 | 68.8 |
| F27 | 50.5 | 227.1 | 77.8 |
| F28 | 29.2 | 213.4 | 86.3 |
| F29 | 26.2 | 243.4 | 89.2 |
| F30 | 33.1 | 220.3 | 85.0 |

TABLE A14
RiboGreen, Pre-lyophilization Samples, Round 3

| | Free RNA | Total RNA | |
|---|---|---|---|
| | Conc. | Conc. | |
| Sample | (μg/mL) | (μg/mL) | % Encap. |
| DP (5° C.) | 11.4 | 980.6 | 98.8 |
| Control | | | |
| FB1 | 3.1 | 227.7 | 98.6 |
| FB2 | 3.3 | 231.9 | 98.6 |
| FB3 | 3.1 | 225.3 | 98.6 |
| FB4 | 3.3 | 218.1 | 98.5 |
| FB5 | 2.8 | 231.9 | 98.8 |
| FB6 | 3.2 | 233.7 | 98.6 |
| FB7 | 3.1 | 237.3 | 98.7 |
| FB8 | 3.1 | 227.1 | 98.6 |
| FB9 | 3.4 | 248.5 | 98.6 |
| FB10 | 3.5 | 242.3 | 98.6 |
| FB11 | 4.1 | 246.0 | 98.3 |
| FB12 | 3.1 | 226.7 | 98.6 |
| FB13 | 3.7 | 246.0 | 98.5 |
| FB14 | 3.6 | 251.0 | 98.6 |
| FB15 | 3.2 | 247.9 | 98.7 |
| FB16 | 3.1 | 244.8 | 98.7 |
| FB17 | 3.1 | 244.3 | 98.7 |
| FB18 | 3.0 | 229.4 | 98.7 |
| FB19 | 2.9 | 248.9 | 98.8 |
| FB20 | 3.0 | 247.6 | 98.8 |
| FB21 | 2.7 | 255.4 | 98.9 |
| FB22 | 4.2 | 254.7 | 98.4 |
| FB23 | 3.1 | 265.1 | 98.8 |
| FB24 | 3.0 | 248.2 | 98.8 |
| FB25 | 2.7 | 238.1 | 98.8 |
| FB26 | 2.6 | 240.7 | 98.9 |
| FB27 | 2.7 | 231.7 | 98.8 |
| FB28 | 2.6 | 229.8 | 98.8 |
| FB29 | 2.8 | 241.3 | 98.8 |
| FB30 | 3.0 | 242.6 | 98.7 |

TABLE A15
RiboGreen, Lyo-RNAse-Free Water
Reconstituted Samples, Round 3

| | Free RNA | Total RNA | | |
|---|---|---|---|---|
| | Conc. | Conc. | | % Encap. |
| Sample | (μg/mL) | (μg/mL) | % Encap. | Rank |
| FB1 | 44.5 | 177.6 | 75.0 | 27 |
| FB2 | 16.7 | 112.7 | 85.2 | 17 |
| FB3 | 36.8 | 200.0 | 81.6 | 20 |
| FB4 | 18.7 | 193.3 | 90.3 | 7 |
| FB5 | 42.9 | 190.3 | 77.5 | 25 |
| FB6 | 34.6 | 187.9 | 81.6 | 21 |
| FB7 | 25.3 | 196.4 | 87.1 | 13 |
| FB8 | 41.4 | 196.4 | 78.9 | 24 |
| FB9 | 27.8 | 218.2 | 87.3 | 12 |
| FB10 | 18.1 | 221.7 | 91.8 | 6 |
| FB11 | 12.5 | 198.1 | 93.7 | 3 |
| FB12 | 21.6 | 210.6 | 89.7 | 8 |
| FB13 | 15.5 | 214.1 | 92.7 | 4 |
| FB14 | 11.3 | 216.9 | 94.8 | 1 |
| FB15 | 23.9 | 224.5 | 89.4 | 10 |
| FB16 | 79.5 | 180.7 | 56.0 | 30 |
| FB17 | 73.2 | 183.1 | 60.0 | 29 |
| FB18 | 45.1 | 193.3 | 76.6 | 26 |
| FB19 | 28.0 | 193.3 | 85.5 | 16 |
| FB20 | 39.9 | 212.3 | 81.2 | 23 |
| FB21 | 22.3 | 197.4 | 88.7 | 11 |
| FB22 | 56.4 | 187.3 | 69.9 | 28 |
| FB23 | 30.5 | 219.5 | 86.1 | 14 |
| FB24 | 31.8 | 205.8 | 84.5 | 18 |
| FB25 | 27.7 | 194.5 | 85.7 | 15 |
| FB26 | 20.9 | 198.2 | 89.5 | 9 |
| FB27 | 32.5 | 188.9 | 82.8 | 19 |
| FB28 | 35.8 | 192.0 | 81.4 | 22 |
| FB29 | 10.3 | 191.3 | 94.6 | 2 |
| FB30 | 15.2 | 201.9 | 92.5 | 5 |

TABLE A16
RiboGreen, Lyo-RNAse-Free Water Reconstituted
Samples, Ranked by % Encapsulation, Round 3

| | Free RNA | Total RNA | | |
|---|---|---|---|---|
| | Conc. | Conc. | | % Encap. |
| Sample | (μg/mL) | (μg/mL) | % Encap. | Rank |
| FB14 | 11.3 | 216.9 | 94.8 | 1 |
| FB29 | 10.3 | 191.3 | 94.6 | 2 |
| FB11 | 12.5 | 198.1 | 93.7 | 3 |
| FB13 | 15.5 | 214.1 | 92.7 | 4 |
| FB30 | 15.2 | 201.9 | 92.5 | 5 |
| FB10 | 18.1 | 221.7 | 91.8 | 6 |
| FB4 | 18.7 | 193.3 | 90.3 | 7 |
| FB12 | 21.6 | 210.6 | 89.7 | 8 |
| FB26 | 20.9 | 198.2 | 89.5 | 9 |
| FB15 | 23.9 | 224.5 | 89.4 | 10 |
| FB21 | 22.3 | 197.4 | 88.7 | 11 |
| FB9 | 27.8 | 218.2 | 87.3 | 12 |
| FB7 | 25.3 | 196.4 | 87.1 | 13 |
| FB23 | 30.5 | 219.5 | 86.1 | 14 |
| FB25 | 27.7 | 194.5 | 85.7 | 15 |
| FB19 | 28.0 | 193.3 | 85.5 | 16 |
| FB2 | 16.7 | 112.7 | 85.2 | 17 |
| FB24 | 31.8 | 205.8 | 84.5 | 18 |
| FB27 | 32.5 | 188.9 | 82.8 | 19 |
| FB3 | 36.8 | 200.0 | 81.6 | 20 |
| FB6 | 34.6 | 187.9 | 81.6 | 21 |
| FB28 | 35.8 | 192.0 | 81.4 | 22 |
| FB20 | 39.9 | 212.3 | 81.2 | 23 |
| FB8 | 41.4 | 196.4 | 78.9 | 24 |
| FB5 | 42.9 | 190.3 | 77.5 | 25 |
| FB18 | 45.1 | 193.3 | 76.6 | 26 |
| FB1 | 44.5 | 177.6 | 75.0 | 27 |
| FB22 | 56.4 | 187.3 | 69.9 | 28 |
| FB17 | 73.2 | 183.1 | 60.0 | 29 |
| FB16 | 79.5 | 180.7 | 56.0 | 30 |

TABLE A17
RiboGreen, Standard Curves, Pre-lyophilization Samples Round 1

| | Accep- | Mea- | | Accep- | Mea- |
|---|---|---|---|---|---|
| Standard | tance | sured | Standard | tance | sured |
| Set 1 | Criteria | Values | Set 2 | Criteria | Values |
| Standard | >0.975 | >0.995 | Standard | >0.975 | >0.995 |
| Curve R² | | | Curve R² | | |
| Standard % | ≤20% | <2.9% | Standard % | ≤20% | <5.1% |
| RSD of 2 | | | RSD of 2 | | |
| replicates | | | replicates | | |
| Standard | TE Curve: y = | | Standard | TE Curve: y = | |
| Curve | 296.54x + 4.5676 | | Curve | 301.69x + 4.0823 | |
| Equation | TX Curve: y = | | Equation | TX Curve: y = | |
| | 271.91x + 1.3341 | | | 261.25x + 3.1302 | |
| | Accep- | Mea- | | Accep- | Mea- |
| Standard | tance | sured | Standard | tance | sured |
| Set 3 | Criteria | Values | Set 4 | Criteria | Values |
| Standard | >0.975 | >0.990 | Standard | >0.975 | >0.996 |
| Curve R² | | | Curve R² | | |
| Standard % | ≤20% | <5.2% | Standard % | ≤20% | <2.7% |
| RSD of 2 | | | RSD of 2 | | |
| replicates | | | replicates | | |

| | | | |
|---|---|---|---|
| Standard | TE Curve: y = | Standard | TE Curve: y = |
| Curve | 283.35x + 7.9903 | Curve | 296.81x + 3.484 |
| Equation | TX Curve: y = | Equation | TX Curve: y = |
| | 266.85x + 3.526 | | 264.93x + 2.1919 |

TABLE A18
RiboGreen, Standard Curves, Lyophilization Samples Round 1

| | Accep- | Mea- | | Accep- | Mea- |
|---|---|---|---|---|---|
| Standard | tance | sured | Standard | tance | sured |
| Set 1 | Criteria | Values | Set 2 | Criteria | Values |
| Standard | >0.975 | >0.999 | Standard | >0.975 | >0.999 |
| Curve R² | | | Curve R² | | |
| Standard % | ≤20% | <2.6% | Standard % | ≤20% | <2.7% |
| RSD of 2 | | | RSD of 2 | | |
| replicates | | | replicates | | |
| Standard | TE Curve: y = | | Standard | TE Curve: y = | |
| Curve | 316.44x − 1.8744 | | Curve | 319.52x − 0.8618 | |
| Equation | TX Curve: y = | | Equation | TX Curve: y = | |
| | 276.14x − 0.9037 | | | 275.25x − 0.5433 | |
| | Accep- | Mea- | | Accep- | Mea- |
| Standard | tance | sured | Standard | tance | sured |
| Set 3 | Criteria | Values | Set 4 | Criteria | Values |
| Standard | >0.975 | >0.999 | Standard | >0.975 | >0.998 |
| Curve R² | | | Curve R² | | |
| Standard % | ≤20% | <2.7% | Standard % | ≤20% | <4.5% |
| RSD of 2 | | | RSD of 2 | | |
| replicates | | | replicates | | |

| | | | |
|---|---|---|---|
| Standard | TE Curve: y = | Standard | TE Curve: y = |
| Curve | 322.68x − 2.0813 | Curve | 321.27x + 1.998 |
| Equation | TX Curve: y = | Equation | TX Curve: y = |
| | 276.51x − 0.9131 | | 278.21x − 0.2791 |

TABLE A19
RiboGreen, Standard Curves, Pre-lyophilization Samples Round 2

| | Accep- | Mea- | | Accep- | Mea- |
|---|---|---|---|---|---|
| Standard | tance | sured | Standard | tance | sured |
| Set 1 | Criteria | Values | Set 2 | Criteria | Values |
| Standard | >0.975 | >0.997 | Standard | >0.975 | >0.996 |
| Curve R² | | | Curve R² | | |
| Standard % | ≤20% | <2.9% | Standard % | ≤20% | <3.9% |
| RSD of 2 | | | RSD of 2 | | |
| replicates | | | replicates | | |
| Standard | TE Curve: y = | | Standard | TE Curve: y = | |
| Curve | 337.03x + 0.6133 | | Curve | 328.13x + 3.453 | |
| Equation | TX Curve: y = | | Equation | TX Curve: y = | |
| | 255.63x + 2.999 | | | 251.11x + 5.6462 | |
| | Accep- | Mea- | | Accep- | Mea- |
| Standard | tance | sured | Standard | tance | sured |
| Set 3 | Criteria | Values | Set 4 | Criteria | Values |
| Standard | >0.975 | >0.996 | Standard | >0.975 | >0.991 |
| Curve R² | | | Curve R² | | |
| Standard % | ≤20% | <5.4% | Standard % | ≤20% | <5.1% |
| RSD of 2 | | | RSD of 2 | | |
| replicates | | | replicates | | |

| | | | |
|---|---|---|---|
| Standard | TE Curve: y = | Standard | TE Curve: y = |
| Curve | 322.29x + 4.8151 | Curve | 321.03x + 7.3171 |
| Equation | TX Curve: y = | Equation | TX Curve: y = |
| | 249.2x + 4.5573 | | 242.45x + 7.1056 |

TABLE A20
RiboGreen, Standard Curves, Lyo-MQ
Reconstituted Samples Round 2

| | Accep- | Mea- | | Accep- | Mea- |
|---|---|---|---|---|---|
| Standard | tance | sured | Standard | tance | sured |
| Set 1 | Criteria | Values | Set 2 | Criteria | Values |
| Standard | >0.975 | >0.997 | Standard | >0.975 | >0.997 |
| Curve R² | | | Curve R² | | |
| Standard % | ≤20% | <3.9% | Standard % | ≤20% | <7.2% |
| RSD of 2 | | | RSD of 2 | | |
| replicates | | | replicates | | |
| Standard | TE Curve: y = | | Standard | TE Curve: y = | |
| Curve | 363.7x − 1.3867 | | Curve | 372.41x − 1.9977 | |
| Equation | TX Curve: y = | | Equation | TX Curve: y = | |
| | 278.73x + 1.002 | | | 278.01x + 1.0819 | |
| | Accep- | Mea- | | Accep- | Mea- |
| Standard | tance | sured | Standard | tance | sured |
| Set 3 | Criteria | Values | Set 4 | Criteria | Values |
| Standard | >0.975 | >0.998 | Standard | >0.975 | >0.996 |
| Curve R² | | | Curve R² | | |
| Standard % | ≤20% | <5.5% | Standard % | ≤20% | <7.6% |
| RSD of 2 | | | RSD of 2 | | |
| replicates | | | replicates | | |

| | | | |
|---|---|---|---|
| Standard | TE Curve: y = | Standard | TE Curve: y = |
| Curve | 369.48x + 0.5963 | Curve | 359.68x + 5.5513 |
| Equation | TX Curve: y = | Equation | TX Curve: y = |
| | 269.55x + 0.9963 | | 273.01x + 2.6739 |

TABLE A21
RiboGreen, Standard Curves, Lyo-TNS Buffer,
pH 7.5 Reconstituted Samples Round 2

| | Accep- | Mea- | | Accep- | Mea- |
|---|---|---|---|---|---|
| Standard | tance | sured | Standard | tance | sured |
| Set 1 | Criteria | Values | Set 2 | Criteria | Values |
| Standard | >0.975 | >0.998 | Standard | >0.975 | >0.998 |
| Curve R² | | | Curve R² | | |
| Standard % | ≤20% | <3.6% | Standard % | ≤20% | <3.1% |
| RSD of 2 | | | RSD of 2 | | |
| replicates | | | replicates | | |
| Standard | TE Curve: y = | | Standard | TE Curve: y = | |
| Curve | 344.83x − 5.5663 | | Curve | 343.51x − 4.7192 | |
| Equation | TX Curve: y = | | Equation | TX Curve: y = | |
| | 271.19x − 0.5859 | | | 272.74x − 1.4877 | |
| | Accep- | Mea- | | Accep- | Mea- |
| Standard | tance | sured | Standard | tance | sured |
| Set 3 | Criteria | Values | Set 4 | Criteria | Values |
| Standard | >0.975 | >0.998 | Standard | >0.975 | >0.998 |
| Curve R² | | | Curve R² | | |
| Standard % | ≤20% | <5.1% | Standard % | ≤20% | <5.6% |
| RSD of 2 | | | RSD of 2 | | |
| replicates | | | replicates | | |

| | | | |
|---|---|---|---|
| Standard | TE Curve: y = | Standard | TE Curve: y = |
| Curve | 347.54x − 3.9121 | Curve | 348.21x − 1.4017 |
| Equation | TX Curve: y = | Equation | TX Curve: y = |
| | 271.93x − 0.1143 | | 265.92x + 2.5283 |

TABLE A22
RiboGreen, Standard Curves, Pre-lyophilization Samples Round 3

| | Accep- | Mea- | | Accep- | Mea- |
|---|---|---|---|---|---|
| Standard | tance | sured | Standard | tance | sured |
| Set 1 | Criteria | Values | Set 2 | Criteria | Values |
| Standard | >0.975 | >0.997 | Standard | >0.975 | >0.998 |
| Curve R² | | | Curve R² | | |
| Standard % | ≤20% | <4.1% | Standard % | ≤20% | <4.7% |
| RSD of 2 | | | RSD of 2 | | |
| replicates | | | replicates | | |
| Standard | TE Curve: y = | | Standard | TE Curve: y = | |
| Curve | 342.37x − 0.9123 | | Curve | 346.26x + 0.8912 | |
| Equation | TX Curve: y = | | Equation | TX Curve: y = | |
| | 278.32x + 1.5895 | | | 267.47x + 1.5509 | |
| | Accep- | Mea- | | Accep- | Mea- |
| Standard | tance | sured | Standard | tance | sured |
| Set 3 | Criteria | Values | Set 4 | Criteria | Values |
| Standard | >0.975 | >0.998 | Standard | >0.975 | >0.998 |
| Curve R² | | | Curve R² | | |
| Standard % | ≤20% | <4.8% | Standard % | ≤20% | <5.8% |
| RSD of 2 | | | RSD of 2 | | |
| replicates | | | replicates | | |

| | | | |
|---|---|---|---|
| Standard | TE Curve: y = | Standard | TE Curve: y = |
| Curve | 340.32x − 0.1105 | Curve | 336.42x + 1.086 |
| Equation | TX Curve: y = | Equation | TX Curve: y = |
| | 257.16x + 2.6614 | | 260.79x + 1.9737 |

TABLE A23
RiboGreen, Standard Curves, Lyo-RNase-Free
Water Reconstituted Samples Round 3

| | Accep- | Mea- | | Accep- | Mea- |
|---|---|---|---|---|---|
| Standard | tance | sured | Standard | tance | sured |
| Set 1 | Criteria | Values | Set 2 | Criteria | Values |
| Standard | >0.975 | >0.994 | Standard | >0.975 | >0.994 |
| Curve R² | | | Curve R² | | |
| Standard % | ≤20% | <3.1% | Standard % | ≤20% | <17.7% |
| RSD of 2 | | | RSD of 2 | | |
| replicates | | | replicates | | |
| Standard | TE Curve: y = | | Standard | TE Curve: y = | |
| Curve | 351.75x + 4.9211 | | Curve | 342.82x + 1.7801 | |
| Equation | TX Curve: y = | | Equation | TX Curve: y = | |
| | 274.97x + 6.6726 | | | 240.04x + 9.7258 | |
| | Accep- | Mea- | | Accep- | Mea- |
| Standard | tance | sured | Standard | tance | sured |
| Set 3 | Criteria | Values | Set 4 | Criteria | Values |
| Standard | >0.975 | >0.995 | Standard | >0.975 | >0.991 |
| Curve R² | | | Curve R² | | |
| Standard % | ≤20% | <2.7% | Standard % | ≤20% | <10.7% |
| RSD of 2 | | | RSD of 2 | | |
| replicates | | | replicates | | |

| | | | |
|---|---|---|---|
| Standard | TE Curve: y = | Standard | TE Curve: y = |
| Curve | 348.31x + 1.979 | Curve | 345.01x + 1.2758 |
| Equation | TX Curve: y = | Equation | TX Curve: y = |
| | 279.85x + 6.8321 | | 268.54x + 3.2308 |

TABLE A24
RiboGreen, Standard Curves, Post-Dialysis Samples Round 1

| Standard Set 1 | Acceptance Criteria | Measured Values |
|---|---|---|
| Standard Curve R² | >0.975 | >0.999 |
| Standard % RSD of 2 replicates | ≤20% | <4.8% |

| | |
|---|---|
| Standard Curve Equation | TX Curve: y = 293.84x − 2.242 |

TABLE A25
RiboGreen, Standard Curves, Post-Dialysis Samples Round 2

| Standard Set 1 | Acceptance Criteria | Measured Values |
|---|---|---|
| Standard Curve R² | >0.975 | >0.998 |
| Standard % RSD of 2 replicates | ≤20% | <3.3% |

| | |
|---|---|
| Standard Curve Equation | TX Curve: y = 267.84x + 1.4237 |

TABLE A26
RiboGreen, Standard Curves, Post-Dialysis Samples Round 3

| Plate 1 | Acceptance Criteria | Measured Values |
|---|---|---|
| Standard Curve R² | >0.975 | >0.996 |
| Standard % RSD of 2 replicates | ≤20% | <6.8% |

| | |
|---|---|
| Standard Curve Equation | TX Curve: y = 214.45x + 11.38 |

The stability of test sample was evaluated for three rounds of screening by visual appearance, dynamic light scattering (DLS), and RiboGreen RNA quantitation. Formulations containing 9% sucrose, 50 mM NaCl, and 50 mM Tris-HCl consistently displayed elegant, lyophilized cakes across all three rounds. Dextran 40 and iodixanol-containing formulations also produced elegant cakes with few minor bubbles. Conversely, formulations containing sodium benzoate or potassium sorbate without sucrose or iodixanol produced cakes which were collapsed or had many large bubbles. Upon reconstitution, sucrose-containing formulations displayed comparable to slightly increased opalescence in comparison to their pre-lyophilized states across three rounds. Particularly, following reconstitution with RNase-free water in Round 3, sucrose formulations containing arginine and PVA10 were the least opalescent, whereas Dextran 40 formulations displayed the most opalescence.

Across three rounds, the DLS results following lyophilization revealed that formulations containing 9% sucrose and PVA10 showed smaller increases in hydrodynamic diameters compared to other formulations such as those containing iodixanol. In Rounds 1 and 2, formulations without P188 showed larger increases in hydrodynamic diameters after lyophilization than their P188-containing counterparts. In Round 3, Dextran 40 formulations showed larger increases in hydrodynamic diameters after lyophilization than other reconstituted formulations. Sucrose-containing formulations containing arginine and PVA10 were among those with the smallest increase in hydrodynamic diameters compared to their pre-lyophilization states, indicating these excipients optimally assist in the retention of test sample's hydrodynamic morphology.

Analysis of the RiboGreen RNA quantification at each round provided potential excipients for the next round of screening. In Round 2, reconstitution with MQ water resulted in slightly higher percent RNA encapsulation than reconstitution with TNS buffer. Furthermore, NaCl-containing formulations were found to provide improved encapsulation compared to formulations without NaCl in Round 2. Despite reported large increases in hydrodynamic diameters, iodixanol-containing formulations experienced smaller losses of encapsulation following lyophilization. In Round 3, Dextran 40 formulations displayed the largest loss of encapsulation and the greatest increase in hydrodynamic diameters. Formulations containing arginine and PVA10 showed high percentages of encapsulation after reconstitution. High concentrations of Tris-HCl also maintained high percentages of encapsulation yet showed large increases in hydrodynamic diameters after lyophilization. Therefore, arginine and PVA10 together with NaCl are better suited for higher RNA encapsulation following lyophilization compared to other formulations tested, maintaining their advantages to stability as previously observed by DLS and visual appearance. Formulation of 9% sucrose, 50 mM NaCl, 50 mM Tris-HCl, 100 mM arginine, and 0.05% PVA10 provides better stabilization for test sample through lyophilization and subsequent reconstitution by RNase-free water compared to other conditions tested.

In order to determine the improvements in excipient performance as described herein, stabilization during lyophilization was analyzed. Following dialysis, samples undergo spike with respective excipients targeting the various formulations, as shown in Table 21. The total volume of each formulation was targeted at 300 μL. 100 μL samples served as a pre-lyophilization control and analyzed without freezing. Two 96-well plates were prepared for lyophilization, with 100 μL samples in each well.

A conservative lyophilization cycle was used to accommodate sufficient drying of all candidate formulations (Table 20). Following lyophilization, one lyophilized plate was sealed and stored at 5° C. for potential further testing. The other plate was visually inspected in the lyophilized state, and then reconstituted with 100 μL of Milli-Q water. Both pre-lyophilization samples and reconstituted lyophilized samples were analyzed by visual inspection, DLS, and RiboGreen.

TABLE 20
Cycle Parameter

| | | | | Chamber |
|---|---|---|---|---|
| | Temperature | Time | Ramp Rate | Pressure |
| Step | (° C.) | (min) | (° C./min) | (mT) |
| Loading | 5 | N/A | N/A | N/A |
| Freezing | 5 to −50 | 55 | 1 | N/A |
| | −50 | 120 | N/A | N/A |
| | −50 to −20 | 60 | 0.5 | N/A |
| | −20 | 120 | N/A | N/A |
| Primary Drying | −20 | 1000* | N/A | 100 |
| Secondary | −20 to 20 | 80 | 0.5 | 100 |
| Drying | 20 | 600 | N/A | 100 |

TABLE 21
Excipient Screening Matrix

| | Buffer | | Hydrophobic salts/AA | | |
|---|---|---|---|---|---|
| Form # | (50 mM) | Polymers | (100 mM) | NaCl | Sucrose/Iodixanol |
| 1 | HEPES | 0.05% | Sodium Benzoate | 50 mM | None |
| 2 | | PVA10 | Potassium Sorbate | | |
| 3 | | 0.2% | Sodium Benzoate | | |
| 4 | | P188 | Potassium Sorbate | | |
| 5 | Tris-HCl | 0.05% | Potassium Sorbate | | |
| 6 | | PVA10 | Sodium Benzoate | | |
| 7 | | 0.2% | Potassium Sorbate | | |
| 8 | | P188 | Sodium Benzoate | | |
| 9 | HEPES | 0.05% | Potassium Sorbate | None | |
| | | PVA10 | | | |
| 10 | | 0.2% | Sodium Benzoate | | |
| | | P188 | | | |
| 11 | Tris-HCl | 0.05% | Sodium Benzoate | | |
| | | PVA10 | | | |
| 12 | | 0.2% | Potassium Sorbate | | |
| 13 | | P188 | None | 50 mM | 2% Sucrose |
| 14 | | | | | 9% Sucrose |
| 15 | | 0.05% | | | |
| | | PVA10 | | | |
| 16 | | 0.2% | Arginine | | None |
| 17 | | P188 | 100 mM Arginine + 100 | | |
| | | | mM Potassium Sorbate | | |
| 18 | | | 100 mM Proline + 100 mM | | |
| | | | Sodium Benzoate | | |
| 19 | HEPES | 0.05% | Proline | None | 9% Iodixanol |
| 20 | | PVA10 | Arginine | | |
| 21 | | 0.2% | Proline | | 9% Sucrose |
| 22 | | P188 | Arginine | | |
| 23 | Tris-HCl | 0.05% | Proline | | 9% Iodixanol |
| 24 | | PVA10 | Arginine | | |
| 25 | | 0.2% | Proline | | 9% Sucrose |
| 26 | | P188 | Arginine | | |
| 27 | | | Proline | 50 mM | 2% Sucrose |
| 28 | | | | | 20% Sucrose |
| 29 | | | Arginine | | 2% Sucrose |
| 30 | | | | | 9% Sucrose |

Test sample was dialyzed against two base buffers (100 mM HEPES and 100 mM Tris, pH adjusted to 7.4) using Thermo Scientific™ Slide-A-Lyzer™, 20 kDa MWCO dialysis cassettes. At least a 10,000-fold buffer exchange was performed. Following dialysis, the pH of the dialyzed bulk DS is confirmed. RiboGreen analysis was performed to determine the Total RNA concentration of each formulation. Each dialyzed DP pool was diluted to 0.5 mg/mL with corresponding base buffers (100 mM HEPES or 100 mM Tris, at pH 7.4) and filtered. Excipient stock solutions were prepared in water and filtered, as shown in Table 22. The pH of the amino acid stock solutions is adjusted to pH 7.4. The 2× excipient solutions were prepared by combining all the necessary excipients for each formulation listed in Table 21. The 2× stock solutions are then spiked into the dialyzed formulations to reach the excipient parameters listed in Table 21 at 0.25 mg/mL test sample concentration.

TABLE 22
2 X Excipient Stock Solutions

| | | | Volume in 1 mL 2x | |
|---|---|---|---|---|
| Reagent | Stock Con. | Target Con. | Stock Sol'n | |
| Poloxamer 188 | 20% (w/v) | 0.20% | 20 | μL |
| PVA 10 | 4% (w/v) | 0.05% | 25 | μL |
| Sodium Benzoate | 2M | 0.1M | 100 | μL |
| Potassium | 2M | 0.1M | 100 | μL |
| Sorbate | | | | |
| Sucrose | 100% (w/v) | 2%, 9%, 20% | 40 μL, 180 μL, 400 μL | |
| Iodixanol | 60% | 9% | 300 | μL |
| Proline | 1M | 0.1M | 200 | μL |
| Arginine | 1M | 0.1M | 200 | μL |
| NaCl | 2.5M | 0.05M | 40 | μL |

A preparation was prepared using 100 μL samples of each formulation which served as a pre-lyophilization control and was analyzed without freezing. Additionally, two 96-well plates were prepared for lyophilization, with 100 μL samples in each well. A conservative lyophilization cycle was used to accommodate sufficient drying of all candidate formulations. Following lyophilization, one lyophilized plate was sealed and stored at 5° C. for further testing. The other plate was visually inspected in the lyophilized state, and then reconstituted with 100 μL of Milli-Q water. Both pre-lyophilization samples and reconstituted lyophilized samples were analyzed by visual inspection, DLS, and RiboGreen.

Excipient screening during Round 2 was performed to assess excipients that can stabilize test sample during lyophilization. Following dialysis, samples may undergo a spike with respective excipients targeting the various formulations, as shown in Table 24. Thirty (30) formulations were prepared, targeting a 0.25 mg/mL final concentration of test sample. The total volume of each formulation was 400 μL. 100 μL samples serve as a pre-lyophilization control and were analyzed without freezing. Of note, before dialysis, the drug product was aliquoted and kept at 5° C. without refreezing. The DP control was analyzed together with the pre-lyophilization controls. Three 96-well plates were prepared for lyophilization, with 100 μL samples in each well. A revised lyophilization cycle is used to accommodate sufficient drying of all candidate formulations (Table 23). Following lyophilization, one lyophilized plate was sealed and stored at 5° C. for potential further testing. The other two plates were visually inspected in the lyophilized state, and then reconstituted with 100 μL of Milli-Q water and 100 μL of 12.5 mM Tris, 12.5 mM NaCl, 2.25% sucrose, pH 7.5, respectively. Both pre-lyophilization samples and reconstituted lyophilization samples were analyzed by visual inspection, DLS, and RiboGreen.

TABLE 23
Cycle Parameter

| | | | | Chamber |
|---|---|---|---|---|
| | Temperature | Time | Ramp Rate | Pressure |
| Step | (° C.) | (min) | (° C./min) | (mT) |
| Loading | 5 | N/A | N/A | N/A |
| Freezing | 5 to −50 | 55 | 1 | N/A |
| | −50 | 120 | N/A | N/A |
| | −50 to −30 | 40 | 0.5 | N/A |
| | −30 | 120 | N/A | N/A |
| Primary Drying | −30 | 1000* | N/A | 100 |
| Secondary | −30 to 5 | 70 | 0.5 | 100 |
| Drying | 5 | 1200 | N/A | 100 |

TABLE 24
Excipient Screening Matrix

| | | | | | | Sucrose/ | |
|---|---|---|---|---|---|---|---|
| | Buffer | PVA10 | P188 | Amino | Amino Acid | Iodixanol | NaCl |
| Form # | (50 mM) | (%) | (%) | Acid | Con. (M) | (9%) | (mM) |
| 1 | Tris-HCl | 0.1 | 0.4 | Arginine | 0.05 | Sucrose | NA |
| 2 | | 0.1 | 0.2 | Proline | 0.05 | Sucrose | NA |
| 3 | | 0.05 | 0.4 | Proline | 0.1 | Sucrose | NA |
| 4 | | 0.05 | NA | Arginine | 0.1 | Sucrose | NA |
| 5 | | 0.035 | 0.2 | Proline | 0.1 | Sucrose | NA |
| 6 | | 0.035 | NA | Proline | 0.1 | Sucrose | NA |
| 7 | | 0.1 | 0.2 | Arginine | 0.05 | Iodixanol | NA |
| 8 | | 0.1 | NA | Proline | 0.1 | Iodixanol | NA |
| 9 | | 0.05 | 0.4 | Arginine | 0.1 | Iodixanol | NA |
| 10 | | 0.05 | 0.2 | Proline | 0.05 | Iodixanol | NA |
| 11 | | 0.035 | 0.4 | Proline | 0.05 | Iodixanol | NA |
| 12 | | 0.035 | NA | Arginine | 0.05 | Iodixanol | NA |
| 13 | | 0.1 | 0.4 | Arginine | 0.05 | Sucrose | 50 |
| 14 | | 0.1 | 0.2 | Proline | 0.05 | Sucrose | 50 |
| 15 | | 0.05 | 0.4 | Proline | 0.1 | Sucrose | 50 |
| 16 | | 0.05 | NA | Arginine | 0.1 | Sucrose | 50 |
| 17 | | 0.035 | 0.2 | Proline | 0.1 | Sucrose | 50 |
| 18 | | 0.035 | NA | Proline | 0.1 | Sucrose | 50 |
| 19 | | 0.1 | 0.2 | Arginine | 0.05 | Iodixanol | 50 |
| 20 | | 0.1 | NA | Proline | 0.1 | Iodixanol | 50 |
| 21 | | 0.05 | 0.4 | Arginine | 0.1 | Iodixanol | 50 |
| 22 | | 0.05 | 0.2 | Proline | 0.05 | Iodixanol | 50 |
| 23 | | 0.035 | 0.4 | Proline | 0.05 | Iodixanol | 50 |
| 24 | | 0.035 | NA | Arginine | 0.05 | Iodixanol | 50 |
| 25 | | NA | 0.2 | Proline | 0.1 | Iodixanol | 50 |
| 26 | | NA | 0.2 | Arginine | 0.1 | Iodixanol | 50 |
| 27 | | NA | NA | Proline | 0.1 | Iodixanol | 50 |
| 28 | | NA | NA | Arginine | 0.1 | Sucrose | 50 |
| 29 | | NA | NA | NA | NA | Iodixanol | 50 |
| 30 | | NA | NA | NA | NA | Sucrose | 50 |

The Experimental procedure included in Round 3 excipient studies were performed using visual, DLS and RiboGreen. Formulation preparation of test sample was dialyzed against base buffers (100 mM Tris, pH adjusted to 7.4) using Thermo Scientific™ Slide-A-Lyzer™, 20 kDa MWCO dialysis cassettes. At least a 10,000-fold buffer exchange was performed. Following dialysis, the pH of the dialyzed bulk DS was confirmed. RiboGreen analysis was performed to determine the Total RNA concentration of each formulation. The dialyzed DP pool was diluted to 0.5 mg/mL with the base buffer and filtered. Excipient stock solutions were prepared in water and filtered, as shown in Table 25. The pH of the amino acid stock solutions was adjusted to a pH 7.4. The 2× excipient solutions were prepared by combining all the necessary excipients for each formulation listed in Table 24. The 2× stock solutions are then spiked into the dialyzed formulations to reach the excipient parameters listed in Table 24 at 0.25 mg/mL test sample concentration.

TABLE 25
2 X Excipient Stock Solutions

| | | | Volume in 1 mL 2x | |
|---|---|---|---|---|
| Reagent | Stock Con. | Target Con. | Stock Solution | |
| Poloxamer 188 | 20% (w/v) | 0.2%, 0.4% | 20 μL, 40 μL | |
| PVA 10 | 4% (w/v) | 0.035%, 0.05%, 0.1% | 17.5 μL, 25 μL, 50 μL | |
| Sucrose | 60% (w/v) | 9% | 300 | μL |
| Iodixanol | 60% | 9% | 300 | μL |
| Proline | 1M | 0.05M, 0.1M | 100 μL, 200 μL | |
| Arginine | 1M | 0.05M, 0.1M | 100 μL, 200 μL | |
| NaCl | 2.5M | 0.05M | 40 | μL |

Following preparation, 100 μL samples of each formulation served as a pre-lyophilization control and were analyzed without freezing. Three 96-well plates were prepared for lyophilization, with 100 μL samples in each well. Following lyophilization, one lyophilized plate was sealed and stored at 5° C. for potential further testing. The other two plates were visually inspected in the lyophilized state, and then reconstituted with 100 μL of two reconstitution buffers, respectively. The two reconstitution buffers are 1) Milli-Q water and 2) 12.5 mM Tris, 12.5 mM NaCl, 2.25% sucrose, pH 7.5. Both pre-lyophilization samples and reconstituted lyophilization samples were analyzed by visual inspection, DLS, and RiboGreen.

Round 3 included the respective excipients targeting the various formulations desired to be utilized for therapeutic formulations within the scope of the described invention. As shown in Table 27, thirty (30) formulations were prepared, targeting a 0.25 mg/mL final concentration of test sample. The total volume of each formulation was 400 μL. 100 μL samples served as a pre-lyophilization control and were analyzed without freezing. Of note, before dialysis, the drug product was aliquoted and kept at 5° C. without refreezing. The DP control was analyzed together with the pre-lyophilization controls. Three 96-well plates were prepared for lyophilization, with 100 μL samples in each well. A revised lyophilization cycle was used to accommodate sufficient drying of all candidate formulations (Table 26).

Following lyophilization, two lyophilized plates were sealed and stored at 5° C. The other plate was visually inspected in the lyophilized state, and then reconstituted with 100 μL of Milli-Q water. Both pre-lyophilization samples and reconstituted lyophilization samples were analyzed by visual inspection, DLS, and RiboGreen.

TABLE 26
Cycle Parameter

| | Temperature | Time | Ramp Rate | Chamber |
|---|---|---|---|---|
| Step | (° C.) | (min) | (° C./min) | Pressure (mT) |
| Loading | 5 | N/A | N/A | N/A |
| Freezing | 5 to −50 | 55 | 1 | N/A |
| | −50 | 120 | N/A | N/A |
| | −50 to −30 | 40 | 0.5 | N/A |
| | −30 | 120 | N/A | N/A |
| Primary Drying | −30 | 1000* | N/A | 100 |
| Secondary | −30 to 5 | 70 | 0.5 | 100 |
| Drying | 5 | 1200 | N/A | 100 |

TABLE 27
Excipient Screening Matrix

| | Tris-HCl | Arginine | PVA10 | Sucrose | NaCl | EDTA | Methionine | Dextran |
|---|---|---|---|---|---|---|---|---|
| Form # | (mM) | (M) | (%) | (%) | (mM) | (mM) | (mM) | 40 (%) |
| 1 | 50 | 0 | 0 | 9 | 50 | 0 | 0 | 0 |
| 2 | 50 | 0 | 0 | 3 | 50 | 0 | 0 | 0 |
| 3 | 100 | 0 | 0 | 9 | 50 | 0 | 0 | 0 |
| 4 | 200 | 0 | 0 | 9 | 50 | 0 | 0 | 0 |
| 5 | 50 | 0 | 0 | 9 | 150 | 0 | 0 | 0 |
| 6 | 50 | 0 | 0 | 9 | 300 | 0 | 0 | 0 |
| 7 | 50 | 0.1 | 0 | 9 | 50 | 0 | 0 | 0 |
| 8 | 50 | 0.2 | 0 | 9 | 50 | 0 | 0 | 0 |
| 9 | 50 | 0.1 | 0.05 | 9 | 50 | 0 | 0 | 0 |
| 10 | 50 | 0.2 | 0.05 | 9 | 50 | 0 | 0 | 0 |
| 11 | 200 | 0.1 | 0.05 | 9 | 50 | 0 | 0 | 0 |
| 12 | 100 | 0.1 | 0.05 | 9 | 50 | 0 | 0 | 0 |
| 13 | 50 | 0.1 | 0.05 | 9 | 150 | 0 | 0 | 0 |
| 14 | 50 | 0.01 | 0.05 | 9 | 300 | 0 | 0 | 0 |
| 15 | 50 | 0.01 | 0.05 | 3 | 50 | 0 | 0 | 0 |
| 16 | 50 | 0 | 0 | 9 | 50 | 0.1 | 50 | 1 |
| 17 | 50 | 0.1 | 0.05 | 9 | 50 | 0.1 | 50 | 1 |
| 18 | 50 | 0 | 0 | 9 | 50 | 0.1 | 50 | 0 |
| 19 | 50 | 0.1 | 0.05 | 9 | 50 | 0.1 | 50 | 0 |
| 20 | 50 | 0 | 0 | 9 | 50 | 0.1 | 0 | 0 |
| 21 | 50 | 0.1 | 0.05 | 9 | 50 | 0.1 | 0 | 0 |
| 22 | Positive Control Buffer | | | | | | | |
| 23 | 50 | 0 | 0 | 9 | 50 | 0 | 0 | 0 |
| 24 | 50 | 0 | 0 | 9 | 50 | 0 | 0 | 0 |
| 25 | 50 | 0.1 | 0.05 | 9 | 50 | 0 | 0 | 0 |
| 26 | 50 | 0.1 | 0.05 | 9 | 50 | 0 | 0 | 0 |
| 27 | 50 | 0 | 0 | 9 | 150 | 0 | 0 | 0 |
| 28 | 50 | 0 | 0 | 9 | 150 | 0 | 0 | 0 |
| 29 | 50 | 0.1 | 0.05 | 9 | 150 | 0 | 0 | 0 |
| 30 | 50 | 0.1 | 0.05 | 9 | 150 | 0 | 0 | 0 |

If no successful candidate is identified, the experiment can be repeated with additional rounds of screening. Up to a total of three (3) rounds of screening including the initial screen were performed.

Test samples in the current formulation were dialyzed against base buffers (100 mM Tris, pH adjusted to 7.4) using Thermo Scientific™ Slide-A-Lyzer™, 20 kDa MWCO dialysis cassettes. At least a 10,000-fold buffer exchange was performed. Following dialysis, the pH of the dialyzed bulk DS was confirmed. RiboGreen analysis was performed to determine the Total RNA concentration of each formulation. The dialyzed DP pool was diluted to 0.5 mg/mL with the base buffer and filtered. Excipient stock solutions were prepared in water and filtered, as shown in Table 28. The pH of the amino acid stock solutions was adjusted to pH 7.4. The 2× excipient solutions were prepared by combining all the necessary excipients for each formulation listed in Table 27. The 2× stock solutions are then spiked into the dialyzed formulations to reach the excipient parameters listed in Table 27 at 0.25 mg/mL test sample concentration.

TABLE 28
2X Excipient Stock Solutions

| Reagent | Stock Con. | Target Con. | Volume in 1 mL 2x Stock Solution | |
|---|---|---|---|---|
| Arginine | 1M | 0.01M, 0.1M, 0.2M | 20 μL, 200 μL, 400 μL | |
| PVA 10 | 4% (w/v) | 0.05% | 25 | μL |
| Sucrose | 60% (w/v) | 3%, 9% | 100 μL, 300 μL | |
| NaCl | 2.5M | 0.05M, 0.15M, 0.3M | 40 μL, 120 μL, 240 μL | |
| EDTA | 0.01M | 0.1 mM | 20 | μL |
| Methionine | 1M | 0.05M | 100 | μL |
| Dextran 40 | 10% | 1% | 200 | μL |

| | | | |
|---|---|---|---|
| Tris-HCl* | 1M | 0.1M, 0.2M | 100 μL, 300 μL |

*Dialysis buffer: 100 mM Tris, pH 7.4

Following preparation, 100 μL samples of each formulation served as a pre-lyophilization control and were analyzed without freezing. Three 96-well plates were prepared for lyophilization, with 100 μL samples in each well. The other plate was visually inspected in the lyophilized state, and then reconstituted with 100 μL of Milli-Q water. Both pre-lyophilization samples and reconstituted lyophilization samples were analyzed by visual inspection, DLS, and RiboGreen.

### Long-Term Stability Study

Four lots of mRNA-LNP were placed on stability, two having an NP ratio of 6 and two having an NP ratio of 9. The difference in NP ratio was achieved by changing the starting lipid concentration. For all lots the mRNA was diluted to 0.22 mg/mL with mRNA dilution buffer (5 mM glutamic acid, 5 mM histidine, 25 mM arginine, 15% sucrose, pH 4), and mixed with either a total lipid concentration of 50.8 mg/mL for NP9 or 20.3 mg/mL for NP6, while maintaining a molar lipid ratio of ionizable lipid:DSPC:Cholesterol:DMG-PEG (58.0:7.0:33.5:1.5). The hydrated pooled was then diluted 3× with 50 mM histidine, 9% Sucrose at pH 6 and incubated for 15 minutes at room temperature. The solution was then diluted 4× with 100 mM tris, 15% sucrose at pH 7.6.

Final pooled mRNA-LNP was ultrafiltered to approximately 1 mg/mL mRNA and diafiltered with 10 volumes of 50 mM tris, 15% sucrose at pH 7.4 and ultrafiltered to approximately 1.5 mg/mL. Pooled product was then diluted 2× with 50 mM tris, 200 mM arginine, 100 mM sodium chloride, 0.2% polyvinyl alcohol, 15% sucrose at pH 7.4. The solution was adjusted to approximately 0.5 mg/mL mRNA with 50 mM tris, 100 mM arginine, 50 mM sodium chloride, 0.1% polyvinyl alcohol, 15% sucrose at pH 7.6. Bulk product was sterile filtered through a 0.22 μm PES membrane.

1 mL aliquots of Lots 230607A and B were filled into 3 mL glass vials and partially stoppered with West Pharmaceutical lyo stoppers. Vials were lyophilized using the cycle described in Table 29.

0.5 mL aliquots of Lots 230523A and B were filled into 3 mL glass vials and partially stoppered with West Pharmaceutical lyo stoppers. Vials were lyophilized using the cycle described in Table 30.

TABLE 29
Lyophilization Cycle Parameters Used for Lots 230607A and B

| | | | | | | Chamber |
|---|---|---|---|---|---|---|
| Step | Temperature (° C.) | | Time | | Ramp Rate | Pressure |
| Loading | 18° | C. | 10 | min | 5° C./min | NA |
| Freezing | 18° C. to 5° C. | | 13 | min | 1° C./min | NA |
| | 5° | C. | 60 | min | NA | NA |
| | 5 to −50° C. | | Maximum | | NA | NA |
| | −50° | C. | 300 | min | NA | NA |
| Annealing | −50 to −10° C. | | 40 | min | 1° C./min | NA |
| | −10° | C. | 180 | min | NA | NA |
| | −10° C. to −50° C. | | 40 | min | 1° C./min | NA |
| | −50° | C. | 180 | min | NA | NA |
| Primary | −50° C. to −30° C. | | 200 | min | 0.1° C./min | 100 mTorr |
| Drying | −30° | C. | 2400 | min | NA | 100 mTorr |
| | −30° C. to −24° C. | | 60 | min | 0.1° C./min | 100 mTorr |
| | −24° | C. | 900 | min | NA | 100 mTorr |
| Secondary | −24° C. to 25° C. | | 490 | min | 0.1° C./min | 100 mTorr |
| Drying | 25° | C. | 1200 | min | NA | 100 mTorr |

TABLE 30
Lyophilization Cycle Parameters Used for Lots 230523A and B

| | | | | | | Chamber |
|---|---|---|---|---|---|---|
| Step | Temperature (° C.) | | Time | | Ramp Rate | Pressure |
| Loading | 18° | C. | NA | | NA | NA |
| Freezing | 18° C. to 5° C. | | 13 | min | 1° C./min | NA |
| | 5° | C. | 30 | min | NA | NA |
| | 5 to −50° C. | | 55 | min | 1° C./min | NA |
| | −50° | C. | 180 | min | NA | NA |
| Annealing | −50 to −15° C. | | 35 | min | 1° C./min | NA |
| | −15° | C. | 180 | min | NA | NA |
| | −15° C. to −50° C. | | 35 | min | 1° C./min | NA |
| | −50° | C. | 180 | min | NA | NA |
| Primary | −50° C. to −30° C. | | 200 | min | 0.1° C./min | 100 mTorr |
| Drying | −30° | C. | 2400 | min | NA | 100 mTorr |
| | −30° C. to −22° C. | | 80 | min | 0.1° C./min | 100 mTorr |
| | −22° | C. | 600 | min | NA | 100 mTorr |
| Secondary | −22° C. to 25° C. | | 470 | min | 0.1° C./min | 100 mTorr |
| Drying | 25° | C. | 600 | min | NA | 100 mTorr |

Following lyophilization, samples were placed on stability at −20° C., 5° C., 25° C., and 40° C.

Lots 230607A and B were reconstituted and analyzed by DLS, encapsulation efficiency, and mRNA purity by CE-LIF at the time points listed in Table 31. Potency was analyzed at TO and 2 week at 40° C., 4 week 25° C., 6 months at −20° C., and 5° C. for lot 230607A and 1 and 6 months at −20° C. and 5° C. for lot 230607B (Table 35).

For the potency method, HepG2 AGL knockout (KO) cells are seeded into a 96-well plate and cultured overnight. On the same day, the mRNA-LNP test samples, reference standard, and assay control are diluted to the target concentration and then incubated with human serum to allow desorption of the PEG lipid from the LNP surface. The next day, serial dilutions of the human serum-treated DP samples are prepared and then incubated with the HepG2 AGL KO cells overnight to allow LNP entry into the cells and translation of the GDE protein from the delivered mRNA. On the third day, the cells are fixed, permeabilized, blocked, and incubated with a specific anti-GDE antibody. On the last day, a secondary antibody labeled with a fluorescent tag (800 nm) along with a CellTag stain (700 nm) used to normalize the cell numbers are added to stain the cells. The final stained 96-well plate is scanned on the LI-COR Odyssey CLx imaging system at 800 nm and 700 nm. The normalized ICW signals are calculated by dividing the GDE expression (800 nm) by the total cell number (700 nm). Finally, dose-response curves are generated for the test samples, reference standard, and control sample. Relative potency is calculated by comparing the EC50 of the test samples to the reference standard using a sigmoidal 4PL curve fitting model in the PLA software.

Lots 230523A and B were analyzed by DLS, encapsulation efficiency, mRNA purity by CE-LIF at the time point listed in Table 32. Potency was analyzed at TO and 1 week at 40° C., 6 months at −20° C., 5° C. and 25° C. (Table 35).

TABLE 31
Stability testing plan for lots 230607A and B

| | | Purity by CE-LIF | | | | DLS | | | | | | | |
|---|---|---|---|---|---|---|---|---|---|---|---|---|---|
| | | 40° C. | 25° C. | 5° C. | −20° C. | 40° C. | 25° C. | 5° C. | −20° C. | Encapsulation Efficiency | | | |
| T0 | | x | | x | | x | | | | 40° C. | 25° C. | 5° C. | −20° C. |
| 1 | day | x | x | x | | | | | | x | x | x | |
| 2 | days | x | x | x¹ | | | | | | x | x | x | |
| 1 | week | x | x | x | | | | | | x | x | x | |
| 2 | weeks | x | x | x | | x | | | | | | | |
| 4 | weeks | | x | | | | x | | | | | | |
| 3 | months | | | x | x | | | | | | | | |
| 6 | months | | | x | x | | | x | x | | | x | x |

¹Lot 230607B 5° C. 2 week time point was not tested for Purity by CE-LIF

TABLE 32
Stability testing plan for lots 230523A and B

| | | | |
|---|---|---|---|
| 40° C. | 25° C. | 5° C. | −20° C. |

| | |
|---|---|
| T0 | x |

| | | | | | |
|---|---|---|---|---|---|
| 1 | week | x | x | | |
| 2 | weeks | | x | | |
| 3 | months | | x | | |
| 4 | months | | | x | x |
| 6 | months | | x | x | x |

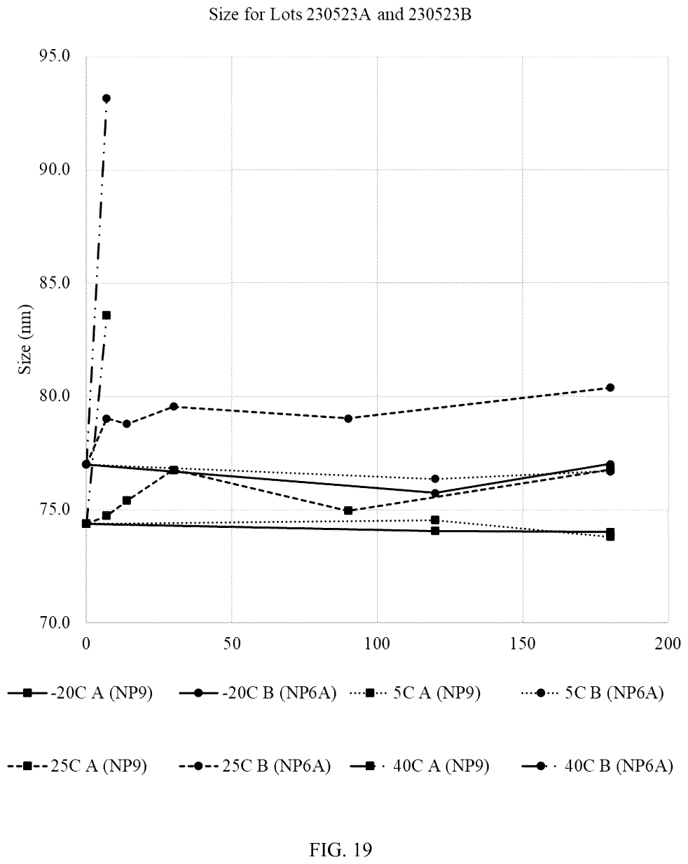

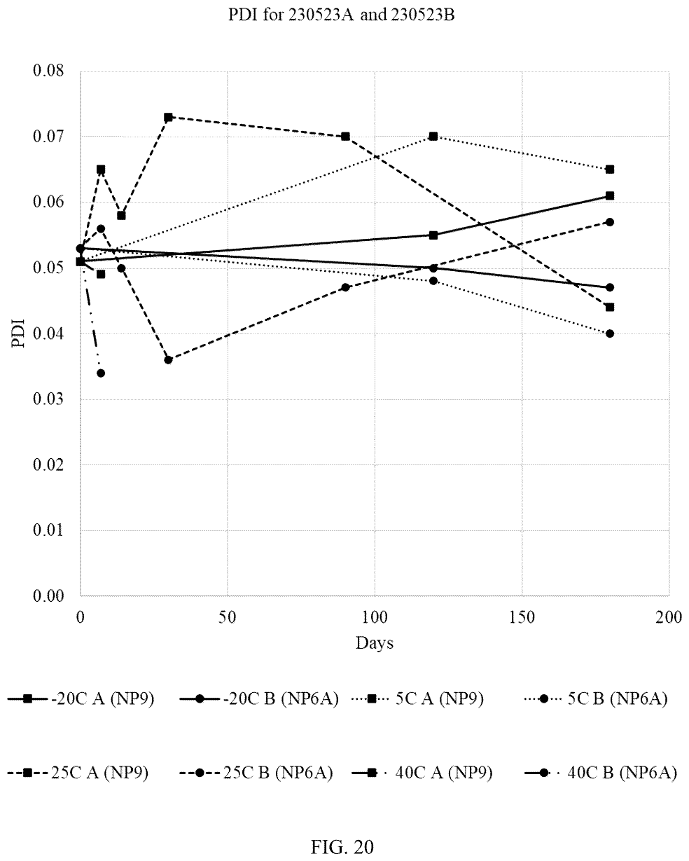

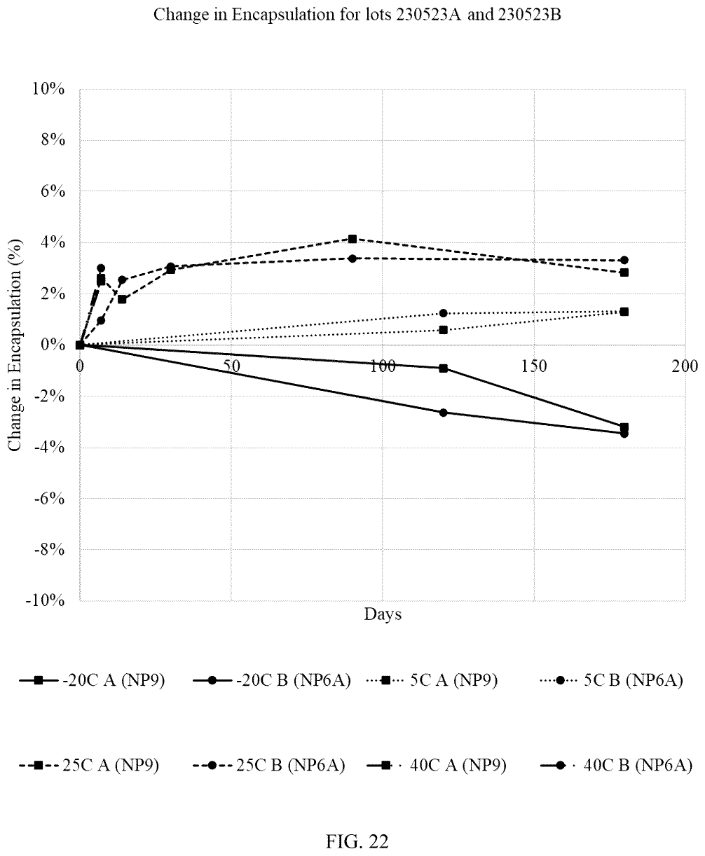

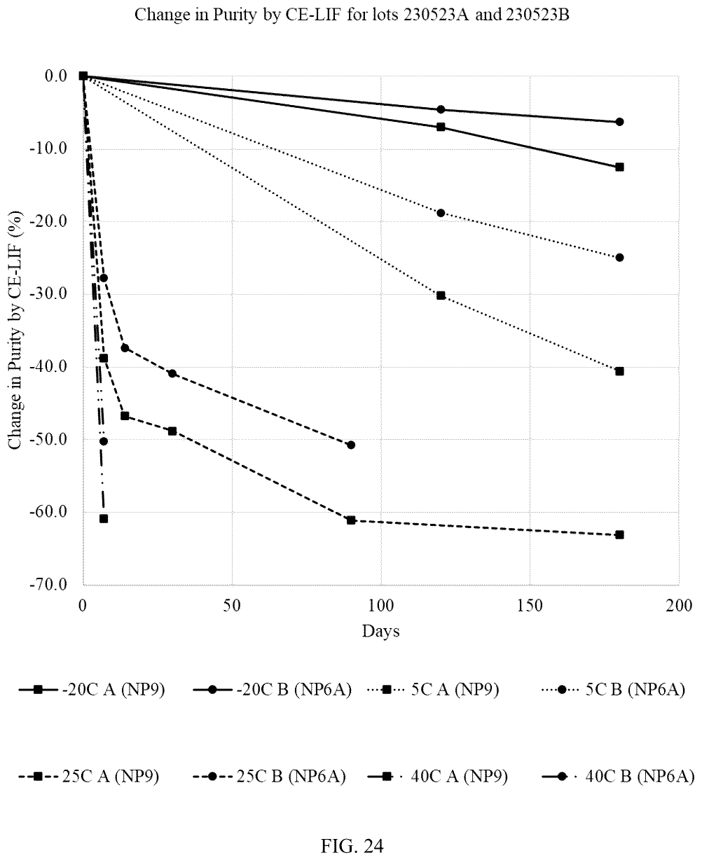

Lots 230523A and B remained stable by polydispersity index [PDI] (FIG. 20) and encapsulation efficiency (FIG. 22) for 6 months at 5° C., 6 months −20° C., 6 months at 25° C. and 2 weeks at 40° C. There is a slight increase in size observed for lots stored 1 week at 40° C. (FIG. 19) but all other conditions remained stable. FIG. 24 shows that there's an increasing loss of purity with increasing temperature storage with all conditions showing a loss of purity, but it appears to be independent of NP ratio.

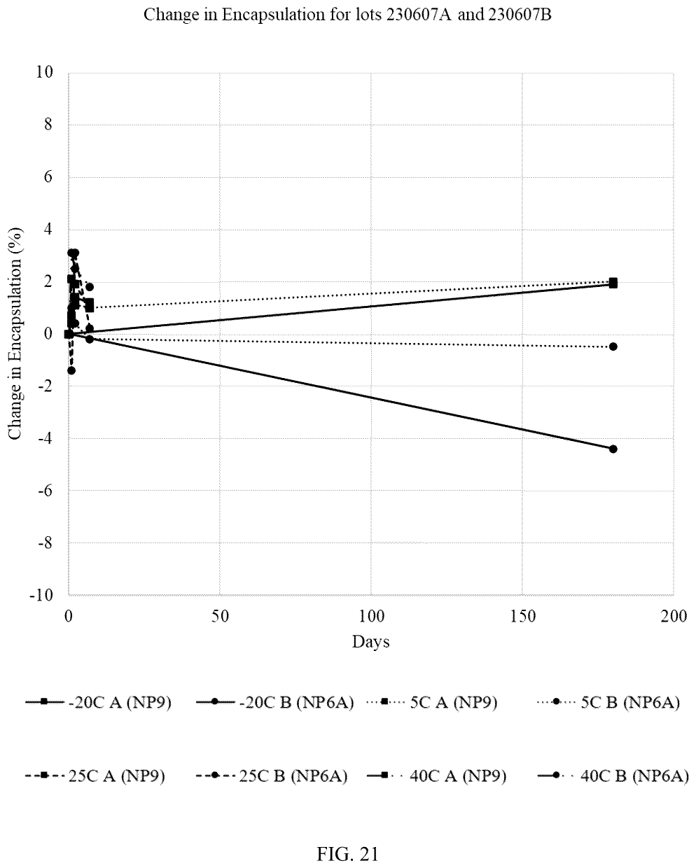

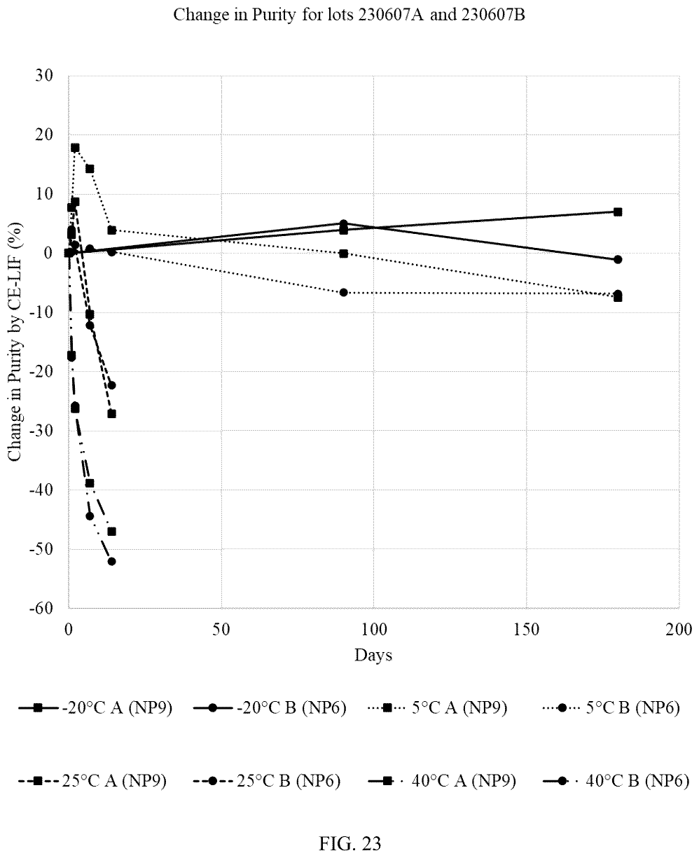

Lots 230607A and B size remained stable after 1 week at 40° C., 1 weeks at 25° C., and 6 months at 5° C. and −20° C. (FIG. 21 and Table 33) and by PDI after 2 weeks at 40° C., 1 month at 25° C., 6 months at 5° C., and 6 months at −20° C. (Table 34). Purity decreased under accelerate and stress storage conditions, 25° C. and 40° C. respectively, with a higher rate of degradation observed at 40° C. Purity remained unchanged after 6 months at 5° C. and −20° C. (FIG. 23).

It appears that the stability of the mRNA-LNP particle is dependent on the lyophilization cycle as lots 230607A and B have a slower rate of degradation compared to lots 230523A and B.

TABLE 33
Change in size from T0 for lots 230607

| | A (NP9) | B (NP6) |
|---|---|---|
| | [nm] | [nm] |
| 6 M −20 C. | 6 | −2 |
| 6 M 5 C. | 12 | 3 |
| 1 M 25 C. | 10 | 11 |
| 2 W 40 C. | 16 | 17 |

TABLE 34
Change in PDI from T0 for lots 230607

| | A (NP9) | B (NP6) |
|---|---|---|
| 6 M −20 C. | −0.01 | 0.01 |
| 6 M 5 C. | 0.02 | 0.01 |
| 1 M 25 C. | 0.00 | 0.00 |
| 2 W 40 C. | −0.03 | −0.01 |

TABLE 35
Potency from T0 for lots 230607A and B and 230523A and B

| | | −20 C. | −20 C. | 5 C. | 5 C. | 25 C. | 25 C. | 40 C. | 40 C. |
|---|---|---|---|---|---|---|---|---|---|
| | 0 | 1 M | 6 M | 1 M | 6 M | 28 D | 6 M | 1 W | 2 W |
| 230607A(NP9) | 137 | 267 | 408 | 206 | 391 | 115 | | | 13 |
| 230607B (NP6) | 133 | | 287 | | 306 | | | | |
| 230523A (NP9) | 96 | | 92 | | 51 | | 0 | 0 | |
| 230523B (NP6) | 72 | | 86 | | 49 | | 0 | 0 | |

### ENUMERATED EMBODIMENTS

Although the present disclosure has been described with respect to one or more particular embodiments, it will be understood that other embodiments of the present disclosure may be made without departing from the scope of the present disclosure.

Further examples and embodiments of the present disclosure are provided in the enumerated clauses which follow:

1. A composition comprising a cargo molecule and lipid nanoparticle, wherein the composition is made using the method of any one of clauses 22-88.

2. The composition of clause 1, wherein the cargo molecule comprises a nucleic acid.

3. The composition of clause 2, wherein the nucleic acid comprises a DNA or an RNA, or an antisense oligonucleotide (ASO).

4. The composition of clause 3, wherein the nucleic acid comprises a miRNA, mRNA, siRNA, ASO, SAM, circular RNA, or a combination thereof.

5. The composition of clause 3, wherein the RNA is mRNA.

6. The composition of any one of clauses 2-5, wherein the nucleic acid is from about 14 nucleotides to about 15,000 nucleotides in length.

7. The composition of any one of clauses 1-6, wherein a total lipid to nucleic acid weight ratio in the composition is about 50:1 to about 4:1, or 40:1 to about 25:1, or 35:1 to about 20:1, or 20:1 to about 10:1, or 15:1 to about 4:1.

8. The composition of any one of clauses 1-7, wherein the lipid nanoparticle in the composition after being lyophilized and then reconstituted is increased in size between 3-18% relative to the size of the lipid nanoparticle prior to lyophilization and reconstitution.

9. The composition of any one of clauses 1-7, wherein the lipid nanoparticle in the composition after being lyophilized and then reconstituted is increased in size no more than 17% relative to the size of the lipid nanoparticle prior to lyophilization and reconstitution.

10. The composition of any one of clauses 2-9, wherein the composition after lyophilization comprises a nucleic acid encapsulation efficiency (EE) of at least 70%.

11. The composition of any one of clauses 2-9, wherein the composition after lyophilization comprises a nucleic acid encapsulation efficiency (EE) of at least 80%.

12. The composition of any one of clauses 2-9, wherein the composition after lyophilization comprises a nucleic acid encapsulation efficiency (EE) of at least 85%.

13. The composition of any one of clauses 2-9, wherein the composition after lyophilization comprises a nucleic acid encapsulation efficiency (EE) of at least 90%.

14. The composition of any one of clauses 1-13, wherein the cargo molecule comprises mRNA with a concentration of at least 0.05 mg/ml.

15. The composition of clause 14, wherein the mRNA is combined with a dilution buffer comprising Ala, Arg, His, Asp, Pro, Glu, Met, Thr, or any combination thereof.

16. The composition of clause 15, wherein the dilution buffer comprises at least 2 amino acids selected from a group comprising Ala, Arg, His, Asp, Pro, Glu, Met, Thr, or a combo thereof, wherein one of the amino acids is positively charged and one amino acid is negatively charged.

17. The composition of any one of clauses 1-16, wherein the composition comprises an excipient in a weight ratio of excipient to cargo molecule of about 25:1 to 400:1.

18. The composition of any one of clauses 1-17, wherein the excipient is in a weight ratio of excipient to cargo molecule of about 2:1 to about 10:1.

19. The composition of any one of clauses 1-17, wherein the excipient is in a weight ratio of excipient to cargo molecule of about 3:1 to about 8:1.

20. The composition of any one of clauses 1-17, wherein the excipient is in a weight ratio of excipient to cargo molecule of about 1:1 to about 12:1.

21. The composition of any one of clauses 1-20, wherein the excipient is selected from: citrate, tris, arginine, NaCl, sucrose, PVA, phosphate, HEPES, trehalose, KCl, acetate, bis-tris, histidine, glucose, lactose, raffinose, alanine, asparagine, proline, glutamic acid, methionine, threonine, and combinations thereof.

22. A method of making a composition comprising a lipid nanoparticle and a cargo molecule disposed therein, wherein the composition is capable of being lyophilized, the method comprising:
a. combining an aqueous solution of the cargo molecule and a lipid composition with an ionizable organic solvent solution, thereby forming a lipid/cargo molecule suspension.

23. The method of clause 22, wherein the aqueous solution and/or lipid composition comprises an organic solvent.

24 The method of any one of clauses 22-23, wherein the cargo molecule comprises a nucleic acid.

25. The method of any one of clauses 22-24, wherein the nucleic acid comprises a DNA or an RNA, or an ASO.

26. The method of clause 25, wherein the nucleic acid comprises a miRNA, mRNA, SiRNA, ASO, SAM, circular RNA, or a combination thereof.

27. The method of any one of clauses 25-26, wherein the RNA is mRNA.

28. The method of any one of clauses 22-27, wherein the lipid composition comprises an ionizable lipid composition.

29. The method of any one of clauses 22-28, wherein the organic solvent comprises at least one of DSPC, cholesterol, PEG-DMG, an ionizable lipid, and combinations thereof.

30. The method of any one of clauses 22-29, further comprising the step of adding an excipient to the aqueous solution of the cargo molecule prior to being combined with the lipid composition.

31. The method of any one of clauses 22-29, further comprising the step of adding an excipient to the lipid/cargo molecule suspension.

32. The method of any one of clauses 30-31, wherein the excipient is selected from citrate, tris, arginine, NaCl, sucrose, PVA, phosphate, HEPES, trehalose, KCl, acetate, bis-tris, histidine, glucose, lactose, raffinose, alanine, asparagine, proline, glutamic acid, methionine, threonine, and combinations thereof.

33. The method of any one of clauses 22-32, wherein the ionizable organic solvent solution comprises at least one of an ionizable lipid, DSPC, cholesterol, PEG-DMG and combinations thereof.

34. The method of clause 33, wherein the ionizable organic solvent solution comprises a N/P molar ratio ≥2.5.

35. The method of clause 33, wherein ionizable organic solvent solution comprises a N/P molar ratio ≥6.0.

36. The method of clause 33, wherein the ionizable organic solvent solution comprises a N/P molar ratio ≥9.0.

37. The method of any one of clauses 23-36, further comprising the step of (b) diluting the lipid/cargo molecule suspension combined with the organic solvent to lower the concentration of organic solvent to ≤10% of the total composition.

38. The method of any one of clauses 23-36, further comprising the step of (b) diluting the lipid/cargo molecule suspension combined with the organic solvent to lower the concentration of organic solvent to ≤3% of the total composition.

39. The method of any one of clauses 22-38, further comprising the step of increasing the pH of the composition to a pH at or above 5.0.

40. The method of clause 39, further comprising the step (b) of clause 37 or 38, wherein the pH is increased after step (b).

41. The method of clause 39, further comprising the step (b) of clause 37 or 38, wherein the pH is increased prior to step (b).

42. The method of any one of clauses 40 or 41, wherein the pH is at least 6.

43. The method of any one of clauses 40 or 41, wherein the pH is between 6.9 and 7.9.

44. A method of lyophilizing a composition comprising the lipid/cargo molecule suspension of any one of clauses 22-43.

45. The method of clause 44, wherein the lyophilized composition comprises the lipid nanoparticle having the cargo molecule disposed therein, thereby forming a lyophilized cargo molecule/LNP particle, the method comprising freezing the lipid/cargo molecule suspension.

46. The method of any one of clauses 44 or 45, further comprising the step of drying the lipid/cargo molecule suspension, wherein the drying step is performed by a process selected from a group comprising spin-freeze drying, continuous freeze-drying, sublimation, desorption, vacuum foam drying, spray drying, evaporation and combination thereof.

47. The method of clause 46, wherein the drying step comprises sublimation to achieve a moisture of ≤5%.

48. The method of any one of clauses 44-47, wherein the potency and/or purity yield of the lyophilized composition is ≥70%.

49. The method of any one of clauses 44-47, wherein the potency and/or purity yield of the lyophilized composition is ≥80%.

50. The method of any one of clauses 44-47, wherein the potency and/or purity yield of the lyophilized composition is ≥90%.

51. The method of any one of clauses 44-47, wherein the potency and/or purity yield of the lyophilized composition is ≥100%.

52. The method of any one of clauses 44-51, wherein the lyophilized cargo molecule/LNP particle size is <100 nm.

53. The method of any one of clauses 44-51, wherein the lyophilized cargo molecule/LNP particle size is <90 nm.

54. The method of any one of clauses 44-51, wherein the lyophilized cargo molecule/LNP particle size is <80 nm.

55. The method of any one of clauses 44-51, wherein the lyophilized cargo molecule/LNP particle size is <75 nm.

56. The method of any one of clauses 44-55, wherein the lyophilized cargo molecule/LNP encapsulation efficiency (EE) is ≥80%.

57. The method of any one of clauses 44-55, wherein the lyophilized cargo molecule/LNP encapsulation efficiency (EE) is ≥90%.

58. The method of any one of clause 44-57, wherein the cargo molecule comprises a therapeutic agent at a starting concentration of ≤2 mg/ml under acidic conditions with a pH under 5, preferably between 3-4.5.

59. The method of any one of clause 44-57, wherein the cargo molecule is an mRNA at a starting concentration of ≤1 mg/ml under acidic conditions with a pH under 5, preferably between 3-4.5.

60. The method of any one of clause 44-57, wherein the cargo molecule is an mRNA at a starting concentration of ≤0.55 mg/ml under acidic conditions with a pH under 5, preferably between 3 and 4.5.

61 The method of any one of clause 44-57, wherein the cargo molecule is an mRNA at a starting concentration of ≤0.22 mg/ml under acidic conditions with a pH under 5, preferably between 3 to 4.5.

62. The method of any one of clauses 22-61, wherein the cargo molecule, in a non-lyophilized state, prior to encapsulation, is dissolved in a buffer under acidic pH conditions below the pKa of the lipid composition and wherein the buffer optionally comprises 50 mM NaCl.

63. The method of any one of clauses 22-61, wherein the cargo molecule, in a non-lyophilized state, prior to encapsulation, is dissolved in a buffer under acidic pH conditions below the pKa of the lipid composition and wherein the buffer comprises a salt between 25 mM and 100 mM, wherein the salt is preferably NaCl.

64 The method of any one of clauses 30-63, wherein the excipient(s) comprises at least one positively charged and at least one negatively charged compound.

65. The method of clause 64, wherein the excipient comprise at least two amino acids selected from Ala, Arg, His, Asp, Pro, Glu, Met, Thr, and combinations thereof.

66. The method of any one of clauses 38-65, wherein the dilution step comprises a first dilution step to increase the pH to about 6 and a second dilution step to increase the pH to between 6.8 and 7.8.

67. The method of clause 66, wherein the first dilution step comprises at least one of phosphate, HEPES, tris, bis-tris, acetate, citrate, NaCl, KCl, glutamic acid, arginine, histidine, methionine, glucose, sucrose, lactose, trehalose, raffinose, and combinations thereof.

68. The method of any one of clauses 66 or 67, wherein the second dilution buffer comprises at least one buffer compound selected from amino acids, sugars, salts and combinations thereof.

69. The method of any one of clauses 22-68, further comprising the step of adding a stabilizer to the aqueous solution of the cargo molecule.

70. The method of clause 69, wherein the stabilizer is added prior to mixing the cargo molecule with the organic solvent and wherein the stabilizer is optionally present in any one of the at least one dilution steps.

71. The method of any one of clauses 30-70, wherein the excipient comprises a hydrophilic polymeric compound.

72. The method of any one of clauses 30-71, wherein the excipient further comprises a plasticizer.

73. A method of preparing a reconstituted composition comprising a lipid nanoparticle having a cargo molecule disposed therein from a lyophilized composition prepared according to the method of any one of clauses 44-72, comprising the step of reconstituting the lyophilized composition with a diluent.

74. The method of clause 73, wherein the lipid nanoparticle in the composition after being lyophilized and then reconstituted is increased in size between 3-18% or to a size ≤18% relative to the size of the lipid nanoparticle prior to lyophilization.

75. The method of clause 73, wherein the lipid nanoparticle in the composition after being lyophilized and then reconstituted is increased in size no more than 17% relative to the size of the lipid nanoparticle prior to lyophilization.

76. The method of any one of clauses 73-75, wherein the cargo molecule comprises an RNA and the encapsulation efficiency (EE) of the RNA is at least 70%.

77. The method of any one of clauses 73-75, wherein the cargo molecule comprises an RNA and the encapsulation efficiency (EE) of the RNA is at least 80%.

78. The method of any one of clauses 73-75, wherein the cargo molecule comprises an RNA and the encapsulation efficiency (EE) of the RNA is at least 85%.

79. The method of any one of clauses 73-75, wherein the cargo molecule comprises an RNA and the encapsulation efficiency (EE) of the RNA is at least 90%.

80. The method of any one of clauses 73-79, wherein the encapsulation efficiency (EE) of the composition after being reconstituted has an average variance of no more than 20% of the lyophilized or aqueous form of the composition.

81. The method of any one of clauses 73-79, wherein the encapsulation efficiency (EE) of the composition after being reconstituted has an average variance of between 10-20% of the lyophilized or aqueous form of the composition.

82. A method of preparing a lyophilized composition comprising lipid nanoparticles with reduced percentage of water in a lipid nanoparticle core, the method comprising:
(a) combining an aqueous solution of a cargo molecule with a lipid composition, thereby forming a lipid/cargo molecule suspension;
(b) mixing the lipid/cargo molecule suspension with an organic solvent;
(c) adding an excipient, wherein the excipient is added before or after the aqueous solution of the cargo molecule is combined with the lipid composition;
(d) diluting the lipid/cargo molecule suspension combined with the organic solvent to lower the concentration of organic solvent to ≤10% of the total composition by volume; thereby forming a lipid nanoparticle composition comprising lipid nanoparticles comprising a shell of the lipid composition and a core comprising the cargo molecule in aqueous solution with the organic solvent;
(e) lyophilizing the lipid nanoparticle composition; and
(f) drying the lipid nanoparticle composition, thereby forming the lyophilized composition with lipid nanoparticles having reduced percentage of water in the lipid nanoparticle core.

83. The method of clause 82, wherein the moisture content of the lyophilized composition is ≤5% by weight.

84. The method of clause 82 or 83, wherein the drying step is performed by a process selected from spin-freeze drying, continuous freeze-drying, sublimation, desorption, vacuum foam drying, spray drying, evaporation, and combinations thereof.

85. A method of preparing a lipid nanoparticle composition comprising a lipid nanoparticle prepared according to the method of any one of clauses 82-84, the method comprising reconstituting the lyophilized composition in a diluent.

86. The method clause 85, wherein the diluent comprises saline, water, 50-500 mM NaCl, arginine, D5W, sucrose, polyvinyl alcohol (PVA), tris, or combinations thereof.

87. The method of any one of clauses 82-86, wherein the drying step occurs at a temperature between 30° C. to −50° C. 88. The method of any one of clauses 82-87, wherein the drying step is performed at a time range of between 10-3000 minutes.

89. The composition of any one of clauses 1-21, wherein the cargo molecule comprises an mRNA encoding amylo-alpha-1, 6-glucosidase, 4-alpha-glucanotransferase (AGL), or a fragment thereof.

90. The composition of clause 89, wherein the composition is for ameliorating, preventing, delaying onset, or treating a disease or condition associated with reduced activity of AGL in a subject.

91. The composition of any one of clauses 1-21, wherein the cargo molecule comprises an mRNA encoding bile salt export pump (BSEP).

92. The composition of clause 91, wherein the composition is for ameliorating, preventing, delaying onset, or treating a disease or condition associated with reduced activity of BSEP in a subject.

The above specification, examples and data provide a complete description of the manufacture and use of the methods and compositions of the invention. Since many embodiments of the invention can be made without departing from the spirit and scope of the invention, the invention resides in the claims hereinafter appended.

What is claimed is:

1. A method of making a composition comprising a lipid nanoparticle and a cargo molecule disposed therein, wherein the composition is capable of being lyophilized, the method comprising:
(a) combining an aqueous solution of the cargo molecule and a lipid composition with an ionizable organic solvent solution, thereby forming a lipid/cargo molecule suspension.

2. The method of claim 1, wherein the aqueous solution and/or lipid composition comprises an organic solvent.

3. The method of claim 1, wherein the cargo molecule comprises a nucleic acid.

4. The method of claim 1, wherein the lipid composition comprises an ionizable lipid composition.

5. The method of claim 1, further comprising the step of adding an excipient to the aqueous solution of the cargo molecule prior to being combined with the lipid composition.

6. The method of claim 1, further comprising the step of adding an excipient to the lipid/cargo molecule suspension.

7. The method of claim 1, wherein the ionizable organic solvent solution comprises a N/P molar ratio ≥2.5.

8. The method of claim 2, further comprising the step of (b) diluting the lipid/cargo molecule suspension combined with the organic solvent to lower the concentration of organic solvent to ≤10% of the total composition.

9. The method of claim 1, further comprising the step of increasing the pH of the composition to a pH at or above 5.0.

10. The method of claim 1, wherein the cargo molecule, in a non-lyophilized state, prior to encapsulation, is dissolved in a buffer under acidic pH conditions below the pKa of the lipid composition.

11. A method of lyophilizing a composition comprising the lipid/cargo molecule suspension of claim 1, wherein the lyophilized composition comprises the lipid nanoparticle having the cargo molecule disposed therein, thereby forming a lyophilized cargo molecule/LNP particle, the method comprising freezing the lipid/cargo molecule suspension.

12. The method of claim 11, further comprising the step of drying the lipid/cargo molecule suspension, wherein the drying step is performed by a process selected from a group comprising spin-freeze drying, continuous freeze-drying, sublimation, desorption, vacuum foam drying, spray drying, evaporation and combination thereof.

13. The method of claim 12, wherein the lyophilized cargo molecule/LNP particle size is <100 nm.

14. The method of claim 12, wherein the cargo molecule comprises a therapeutic agent at a starting concentration of ≤2 mg/ml under acidic conditions with a pH under 5, preferably between 3-4.5.

15. A method of preparing a lyophilized composition comprising lipid nanoparticles with reduced percentage of water in a lipid nanoparticle core, the method comprising:
(a) combining an aqueous solution of a cargo molecule with a lipid composition, thereby forming a lipid/cargo molecule suspension;
(b) mixing the lipid/cargo molecule suspension with an organic solvent;
(c) adding an excipient, wherein the excipient is added before or after the aqueous solution of the cargo molecule is combined with the lipid composition;
(d) diluting the lipid/cargo molecule suspension combined with the organic solvent to lower the concentration of organic solvent to ≤10% of the total composition by volume;
thereby forming a lipid nanoparticle composition comprising lipid nanoparticles comprising a shell of the lipid composition and a core comprising the cargo molecule in aqueous solution with the organic solvent;
(e) lyophilizing the lipid nanoparticle composition; and
(f) drying the lipid nanoparticle composition, thereby forming the lyophilized composition with lipid nanoparticles having reduced percentage of water in the lipid nanoparticle core.

16. The method of claim 15, wherein:
(a) the moisture content of the lyophilized composition is ≤5% by weight;
(b) the drying step occurs at a temperature between 30° C. to −50° C.; and/or
(c) the drying step is performed at a time range of between 10-3000 minutes.

17. A composition comprising a cargo molecule and lipid nanoparticle, wherein the composition is made using the method of claim 1.

18. The composition of claim 17, wherein the cargo molecule comprises a nucleic acid.

19. The composition of claim 18, wherein a total lipid to nucleic acid weight ratio in the composition is about 50:1 to about 4:1, or 40:1 to about 25:1, or 35:1 to about 20:1, or 20:1 to about 10:1, or 15:1 to about 4:1.

20. The composition of claim 19, wherein the cargo molecule comprises mRNA with a concentration of at least 0.05 mg/ml.

21. The composition of claim 20, wherein the mRNA is combined with a dilution buffer comprising Ala, Arg, His, Asp, Pro, Glu, Met, Thr, or any combination thereof.

22. The composition of claim 17, wherein the composition comprises an excipient in a weight ratio of excipient to cargo molecule of about 25:1 to 400:1.

23. The composition of claim 17, wherein the cargo molecule comprises an mRNA encoding amylo-alpha-1, 6-glucosidase, 4-alpha-glucanotransferase (AGL), or a fragment thereof.

24. The composition of claim 17, wherein the cargo molecule comprises an mRNA encoding bile salt export pump (BSEP).
